(19) United States
(12) Patent Application Publication
(10) Pub. No.: US 2026/0092099 A1
(43) Pub. Date: Apr. 2, 2026

(54) COMPOSITIONS AND METHODS FOR ALPHA-1-ANTITRYPSIN DISORDERS

(71) Applicant: Spin Therapeutics, LLC, Berkeley, CA (US)

(72) Inventors: Aaron Sato, Berkeley, CA (US); Mark DeSouza, Berkeley, CA (US)

(73) Assignee: Spin Therapeutics, LLC, Berkeley, CA (US)

(21) Appl. No.: 19/388,528

(22) Filed: Nov. 13, 2025

Related U.S. Application Data
(63) Continuation of application No. 17/289,136, now Pat. No. 12,497,441.
(60) Provisional application No. 62/752,182, filed on Oct. 29, 2018.

(51) Int. Cl. C07K 14/81; A61K 38/00; C07K 14/765

(52) U.S. Cl. CPC C07K 14/8125; C07K 14/765; A61K 38/00; C07K 2319/31

(57) ABSTRACT

Disclosed herein are compositions and methods useful for treating an alpha-1-antitrypsin deficiency.

### CROSS-REFERENCE TO RELATED APPLICATIONS

This application is a continuation of U.S. patent application Ser. No. 17/289,136, filed Apr. 27, 2021, which is the U.S. national stage of International Patent Application No. PCT/US2019/058673, filed Oct. 29, 2019, which claims priority to U.S. Provisional Patent Application No. 62/752,182, filed Oct. 29, 2018, all of which are incorporated by reference herein in their entirety.

### SEQUENCE LISTING

The instant application contains a Sequence Listing which has been submitted electronically in XML format and is hereby incorporated by reference in its entirety. Said XML file, created on Nov. 11, 2025, is named “144368-0102_SL.xml” and is 120,799 bytes in size.

### BACKGROUND OF THE INVENTION

Serpins are a superfamily of proteins with similar structures that were first identified for their ability to inhibit proteases. The name serpin was originally coined because the first serpins to be identified were found to act on chymotrypsin-like serine proteases. As a result, the acronym serpin was chosen to incorporate the first couple of letters of the word(s) serine protease inhibitors.

Serpins are interesting because of their unusual mechanism of action in which they irreversibly inhibit a target protease by undergoing a large conformational change to disrupt an active site on the target protease. Protease inhibition by serpins controls an array of biological processes, including coagulation and inflammation. The mechanism of protease inhibition confers certain advantages but also has drawbacks. One major drawback is that serpins are especially vulnerable to a mutation that can result in serpinopathies, such as protein misfolding and the formation of inactive long-chain polymers.

Alpha-1-antitrypsin or ai-antitrypsin (“A1AT,” “A1A,” or “AAT”) is a protein belonging to the serpin superfamily. It is encoded in humans by the SERPINA1 gene, which has 4 coding exons and 3 untranslated exons and resides on chromosome 14 at q31-31.2. It is also known as alpha₁-proteinase inhibitor or alpha₁-antiproteinase because it inhibits various proteases, not just trypsin. It is a 52-kD glycoprotein that functions as an antiprotease. It has a single chain of 394 amino acids with a signal peptide of 24 amino acids in length attached. As a type of enzyme inhibitor, it protects tissues from enzymes of inflammatory cells, especially neutrophil elastase and proteinase 3 (“PR3”), and has a normal reference range in blood of 1.5-3.5 g/L. The concentration of alpha-1-antitrypsin can rise manyfold upon acute inflammation.

Neutrophil elastase is a serine protease that destroys elastase, the rubberlike macromolecule that provides elastic recoil to the lung. When the blood contains inadequate amounts of alpha-1-antitrypsin or functionally defective alpha-1-antitrypsin, neutrophil elastase is excessively free to break down elastin, degrading the elasticity of the lungs. Degradation of elasticity results in respiratory complications, such as chronic obstructive pulmonary disease.

PR3 is a serine protease enzyme expressed mainly in neutrophil granulocytes. Its exact role in the function of the neutrophil is unknown, but, in human neutrophils, proteinase 3 contributes to the proteolytic generation of antimicrobial peptides.

Alpha-1-antitrypsin deficiency (“AATD”) is a genetic disorder that causes defective production of alpha-1-antitrypsin. AATD is an autosomal recessive disorder caused by reduced serum levels of AATD. The disease leads to a decrease of alpha-1-antitrypsin. The disease often causes lung and liver disease and is referred to as hereditary pulmonary emphysema. AATD is one of the most common lethal hereditary disorders of caucasians of European descent.

There are several forms and degrees of alpha-1-antitrypsin deficiency and the form and degree depend on whether the sufferer has one or two copies of a defective allele. Severe alpha-1-antitrypsin deficiency can cause panacinar emphysema or COPD-like symptoms in adult life in many people with the condition, especially if they are exposed to cigarette smoke. The disorder can also lead to various liver diseases in children and adults, and occasionally can lead to problems that are more unusual. Alpha-1-antitrypsin deficiency usually produces some degree of disability and reduces life expectancy.

Alpha-1-antitrypsin deficiency is treated through avoidance of damaging inhalants and by intravenous infusions of alpha-1-antitrypsin or by transplantation of the liver or lungs. Recombinant versions of alpha-1-antitrypsin are also known but are currently used in medical research more than as treatment. Approved alpha-1-antitrypsin deficiency products are purified from human plasma and require frequent weekly infusion, with high doses (60-120 mg/kg) and have only limited efficacy. Alpha-1-antitrypsin augmentation therapy is not ideal due to poor pharmacokinetics, with peaks and troughs seen in circulating AAT levels, despite administration of high concentrations of plasma-derived AAT.

Some current research is focused on developing long-acting alpha-1-antitrypsin fusions through protein engineering. For example, AAT Fc has been expressed and developed by two groups: Soohyun Kim (Konkuk University, Seoul, Korea; licensed to OmniBio (“Kim/OmniBio molecule”) (See Lee S, Lee Y, Hong K, Hong J, Bae S, Choi J, Jhun H, Kwak A, Kim E, Jo S, Dinarello C A, Kim S. Effect of recombinant α1-antitrypsin Fe-fused (AAT-Fc) protein on the inhibition of inflammatory cytokine production and streptozotocin-induced diabetes. Mol Med. 2013 May 20; 19:65-71. doi: 10.2119/molmed.2012.00308. PubMed PMID: 23552726; PubMed Central PMCID:PMC3667213, which is incorporated by reference in its entirety herein, including any drawings.) and InhibRx (“InhibRx molecule”) (See, for example, U.S. Pat. No. 8,980,266 B2, Serpin Fusion Polypeptides and Methods and Use Thereof, which is incorporated in its entirety herein in its entirety, including any drawings.) Both molecules are N-terminal AAT fusions to IgG1 Fc. The Kim/OmniBio molecule does not inhibit human neutrophil elastase as well as plasma derived AAT, which may be a result of low-pH instability upon elution from protein A chromatography during purification. The InhibRx molecule is reported to inhibit human neutrophil elastase like plasma derived AAT.

Others have modified the glycosylation pattern of AAT by increasing the number of N-glycans (See, for example, Chung H S, Kim J S, Lee S M, Park S J. Additional N-glycosylation in the N-terminal region of recombinant human alpha-1 antitrypsin enhances the circulatory half-life in Sprague-Dawley rats. Glycoconj J. 2016 April; 33(2):201-8. doi: 10.1007/s10719-016-9657-3. Epub 2016 Mar. 7. PubMed PMID: 26947874, which is incorporated by reference in its entirety herein, including any drawings) or adding polysialyation (See, for example, Lindhout T, Iqbal U, Willis L M, Reid A N, Li J, Liu X, Moreno M, Wakarchuk W W. Site-specific enzymatic polysialylation of therapeutic proteins using bacterial enzymes. Proc Natl Acad Sci USA. 2011 May 3; 108(18):7397-402. doi:10.1073/pnas.1019266108. Epub 2011 Apr. 18. PubMed PMID: 21502532; PubMed Central PMCID: PMC3088639, which is incorporated by reference in its entirety herein, including any drawings.) Some have produced an aglycosylated AAT (See, for example, Cantin A M, Woods D E, Cloutier D, Heroux J, Dufour E K, Leduc R. Leukocyte elastase inhibition therapy in cystic fibrosis: role of glycosylation on the distribution of alpha-1-proteinase inhibitor in blood versus lung. J Aerosol Med. 2002 Summer; 15(2):141-8. PubMed PMID: 12184864, which is incorporated by reference in its entirety herein, including any drawings.) Still others have developed a PEGylated aglycosylated AAT to extend half-life (See, for example, Cantin A M, Woods D E, Cloutier D, Dufour E K, Leduc R. Polyethylene glycol conjugation at Cys232 prolongs the half-life of alpha1 proteinase inhibitor. Am J Respir Cell Mol Biol. 2002 December; 27(6):659-65. PubMed PMID: 12444025, which is incorporated by reference in its entirety herein, including any drawings.)

AAT is prone to Met oxidation in the lung and so some groups have constructed mutated AAT variants that are resistant to methionine oxidation but retain activity. In particular, the M351V/M358V mutant is resistant to oxidation (See, for example, Taggart C, Cervantes-Laurean D, Kim G, McElvaney N G, Wehr N, Moss J, Levine R L. Oxidation of either methionine 351 or methionine 358 in alpha 1-antitrypsin causes loss of anti-neutrophil elastase activity. J Biol Chem. 2000 Sep. 1; 275(35):27258-65. PubMed PMID: 10867014, which is incorporated by reference in its entirety herein, including any drawings.) Baek et al. have introduced a disulfide bond (K168C-F189C) in AAT and substantially increased resistance to loop-sheet polymerization (See Baek J H, Im H, Kang U B, Seong K M, Lee C, Kim J, Yu M H. Probing the local conformational change of alpha 1-antitrypsin. Protein Sci. 2007 September; 16(9):1842-50. Epub 2007 Jul. 27. PubMed PMID: 17660256; PubMed Central PMCID: PMC2206966m, which is incorporated by reference in its entirety herein, including any drawings.) Single point mutations (F51L, G117F, K331F, K335A) in AAT have also been created and show increases in stability and resistance to loop-sheet polymerization (See, Dafforn T R, Mahadeva R, Elliott P R, Sivasothy P, Lomas D A. A kinetic mechanism for the polymerization of alpha1-antitrypsin. J Biol Chem. 1999 Apr. 2; 274(14):9548-55. PubMed PMID: 10092640; Parfrey H, Mahadeva R, Ravenhill N A, Zhou A, Dafforn T R, Foreman R C, Lomas D A. Targeting a surface cavity of alpha 1-antitrypsin to prevent conformational disease. J Biol Chem. 2003 Aug. 29; 278(35):33060-6. Epub 2003 Jun. 13. PubMed PMID: 12807889; Gilis D, McLennan H R, Dehouck Y, Cabrita L D, Rooman M, Bottomley S P. In vitro and in silico design of alpha1-antitrypsin mutants with different conformational stabilities. J Mol Biol. 2003 Jan. 17; 325(3):581-9. PubMed PMID: 12498804; and Im H, Seo E J, Yu M H. Metastability in the inhibitory mechanism of human alpha1-antitrypsin. J Biol Chem. 1999 Apr. 16; 274(16):11072-7. PubMed PMID: 10196190, each of which is incorporated by reference herein including any drawings.)

However, despite the above-mentioned progress, additional treatment options are needed.

### SUMMARY OF THE INVENTION

The invention described herein is based, in part, on a recombinant engineered alpha-1-antitrypsin serpin domain fused to a human serum albumin binding domain or human serum albumin domain.

Some embodiments comprise a recombinant protein comprising an alpha-1-antitrypsin serpin domain and a human serum albumin binding domain or a human serum albumin domain. In some embodiments, the alpha-1-antitrypsin serpin domain has a sequence comprising all or part of SEQ ID NO: 1. In some embodiments, the recombinant protein comprises one or more linkers. In some embodiments, the C-terminus of the alpha-1-antitrypsin serpin domain is fused to the N-terminus of the human serum albumin or human serum albumin binding domain in the recombinant protein. Some embodiments comprise a recombinant protein comprising an alpha-1-antitrypsin serpin domain and a human serum albumin domain wherein the alpha-1-antitrypsin serpin domain and a human serum albumin domain are not both wild-type alpha-1-antitrypsin and a wild type human serum albumin.

In some embodiments, the alpha-1-antitrypsin serpin domain has one or more mutations. In some embodiments, the alpha-1-antitrypsin serpin domain retains activity compared to a wild type alpha-1-antitrypsin serpin domain. In some embodiments, the one or more mutations causes resistance to methionine oxidation as compared to wild type alpha-1-antitrypsin. In some embodiments, the one or more mutations comprises M351V and/or M358V with residues being numbered according to SEQ ID NO: 1. In some embodiments, the one or more mutations comprises K168C and F189C with residues being numbered according to SEQ ID NO: 1. In some embodiments, the one or more mutations confer a substantially increased resistance to loop-sheet polymerization as compared to wild type alpha-1-antitrypsin. In some embodiments, the one or more mutations are one or more point mutations selected from F51L, G117F, K331F, or K335A with residues being numbered according to SEQ ID NO: 1. In some embodiments, the alpha-1-antitrypsin serpin domain retains activity against human neutrophil elastase and/or PR3 compared to a wild type alpha-1-antitrypsin serpin domain.

In some embodiments, the one or more mutations comprises a mutation in at least one of the following residue positions according to SEQ ID NO 1: 51, 100, 114, 117, 163, 164, 165, 168, 169, 172, 173, 174, 183, 189, 232, 283, 300, 302, 303, 304, 306, 330, 331, 333, 335, 336, 337, 338, 339, 340, 351, 356, 358, or 361. In some embodiments, the one or more mutations comprises one or more of the following mutations according to SEQ ID NO 1: F51L, L100F, L100C, T114F, G117F, K163T, G164V, T165S, K168C, K168I, K168A, K169V, L172V, L172A, L172C, V173C, K174T, A183V, F189C, F189I, F189V, C232S, S283C, K300A, V302A, L303A, G304A, L306A, S330R, K331F, K331I, K331V, K331T, K331C, V333C, V333A, K335A, K331F, K335G, K335T, A336G, V337C, V337A, L338A, T339S, I340V M351V, I356M, M358I, M358P, M358A, M358R, M358L, M358V, or P361C. In some embodiments, the one or more mutations comprises F51L, G117F, K168C, F189C, C232S, S283C, K331F, K335A, M351V, M358V, M358L, or P361C according to SEQ ID NO: 1. In some embodiments, the one or more mutations are both of K168C and F189C according to SEQ ID NO: 1. In some embodiments, the one or more mutations are both of P361C and S283C according to SEQ ID NO: 1.

In some embodiments, the alpha-1-antitrypsin serpin domain has one or more mutations comprising C232S according to SEQ ID NO: 1. In some embodiments, the alpha-1-antitrypsin serpin domain has one or more mutations comprising M351V and M358V according to SEQ ID NO: 1. In some embodiments, the alpha-1-antitrypsin serpin domain has one or more mutations comprising M351V and M358L according to SEQ ID NO: 1. In some embodiments, the alpha-1-antitrypsin serpin domain has one or more mutations comprising C232S, M351V, and M358V according to SEQ ID NO: 1.

In some embodiments, the alpha-1-antitrypsin serpin domain has one or more mutations comprising F51L, C232S, M351V and M358L according to SEQ ID NO: 1. In some embodiments, the alpha-1-antitrypsin serpin domain has one or more mutations comprising G117F, C232S, M351V and M358L according to SEQ ID NO: 1. In some embodiments, the alpha-1-antitrypsin serpin domain has one or more mutations comprising C232S, K331F, M351V and M358L according to SEQ ID NO: 1. In some embodiments, the alpha-1-antitrypsin serpin domain has one or more mutations comprising C232S, K335A, M351V and M358L according to SEQ ID NO: 1. In some embodiments, the alpha-1-antitrypsin serpin domain has one or more mutations comprising K168C, F189C, C232S, M351V, and M358L according to SEQ ID NO: 1. In some embodiments, the alpha-1-antitrypsin serpin domain has one or more mutations comprising C232S, S283C, M351V, M358L, and P361C according to SEQ ID NO: 1. In some embodiments, the alpha-1-antitrypsin serpin domain has one or more mutations comprising F51L, G117F, C232S, M351V, and M358L according to SEQ ID NO: 1. In some embodiments, the alpha-1-antitrypsin serpin domain has one or more mutations comprising F51L, C232S, K331F, M351V, and M358L according to SEQ ID NO: 1. In some embodiments, the alpha-1-antitrypsin serpin domain has one or more mutations comprising F51L, C232S, K335A, M351V, and M358L according to SEQ ID NO: 1. In some embodiments, the alpha-1-antitrypsin serpin domain has one or more mutations comprising K51L, K168C, F189C, C232S, M351V, and M358L according to SEQ ID NO: 1. In some embodiments, the alpha-1-antitrypsin serpin domain has one or more mutations comprising F51L, C232S, S283C, M351V, M358L, and P361C according to SEQ ID NO: 1. In some embodiments, the alpha-1-antitrypsin serpin domain has one or more mutations comprising G117F, C232S, K331F, M351V, and M358L according to SEQ ID NO: 1. In some embodiments, the alpha-1-antitrypsin serpin domain has one or more mutations comprising G117F, K335A, C232S, M351V, and M358L according to SEQ ID NO: 1. In some embodiments, the alpha-1-antitrypsin serpin domain has one or more mutations comprising G117F, K168C, F189C, C232S, M351V, and M358L according to SEQ ID NO: 1. In some embodiments, the alpha-1-antitrypsin serpin domain has one or more mutations comprising G117F, C232S, S283C, M351V, M358L, and P361C according to SEQ ID NO: 1. In some embodiments, the alpha-1-antitrypsin serpin domain has one or more mutations comprising C232S, K331F, K335A, M351V, and M358L according to SEQ ID NO: 1. In some embodiments, the alpha-1-antitrypsin serpin domain has one or more mutations comprising K168C, F189C, C232S, K331F, M351V, and M358L according to SEQ ID NO: 1. In some embodiments, the alpha-1-antitrypsin serpin domain has one or more mutations comprising C232S, S283C, K331F, M351V, M358L, and P361C according to SEQ ID NO: 1. In some embodiments, the alpha-1-antitrypsin serpin domain has one or more mutations comprising K168C, F189C, C232S, K335A, M351V, and M358L according to SEQ ID NO: 1. In some embodiments, the alpha-1-antitrypsin serpin domain has one or more mutations comprising C232S, S283C, K335A, M351V, M358L, and P361C according to SEQ ID NO: 1. In some embodiments, the alpha-1-antitrypsin serpin domain has one or more mutations comprising K168C, F189C, C232S, S283C, M351V, M358L, and P361C according to SEQ ID NO: 1. In some embodiments, the alpha-1-antitrypsin serpin domain comprises a point mutation K335A according to SEQ ID NO: 1. In some embodiments, the alpha-1-antitrypsin serpin domain comprises a point mutation K331F according to SEQ ID NO: 1. In some embodiments, the alpha-1-antitrypsin serpin domain comprises a point mutation G117F according to SEQ ID NO: 1. In some embodiments, the alpha-1-antitrypsin serpin domain comprises a point mutation S283C according to SEQ ID NO: 1. In some embodiments, the alpha-1-antitrypsin serpin domain comprises a point mutation P361C according to SEQ ID NO: 1.

In some embodiments, the human serum albumin is at least a portion of SEQ ID NO: 3 or SEQ ID NO: 5. In some embodiments, the human serum albumin binding domain is one of SEQ ID NOs: 22-36. In some embodiments, the human serum albumin binding domain comprises one or more CDRs selected from SEQ ID NOs: 6-21. In some embodiments, the human serum albumin or human serum albumin binding domain increases plasma half-life of the alpha-1-antitrypsin serpin domain as compared to wild type alpha-1-antitrypsin. In some embodiments, a number of N-glycans or polysialyation of the recombinant protein in the alpha 1-antitrypsin serpin domain is greater than a wild type alpha-1-antitrypsin serpin domain.

In some embodiments, the human serum albumin comprises one or more mutations. In some embodiments, the one or more mutations are one or more of residues 407, 408, 409, 410, 413, and 414, wherein the one or more residues are numbered according to SEQ ID NO: 5. In some embodiments, the one or more mutations are one or more of L407A, L408V, V409A, R410A, L413Q, and L414Q, wherein the one or more residues are numbered according to SEQ ID NO. 5. In some embodiments, the human serum albumin comprises a portion or a variant of human serum albumin.

In some embodiments, the human serum albumin binding protein comprises an amino acid sequence that is capable of binding to all or a. pari of human serum albumin protein having the sequence set forth in SEQ ID NO: 3 or SEQ ID NO: 5.

In some embodiments, the recombinant protein comprises or consists of a protein having any one of SEQ ID Nos: 64-68 or SEQ ID NO: 71. In some embodiments, the recombinant protein comprises or consists of a protein having SEQ ID NO: 64. In some embodiments, the recombinant protein comprises or consists of a protein having SEQ ID NO: 65. In some embodiments, the recombinant protein comprises or consists of a protein having SEQ ID NO: 66. In some embodiments, the recombinant protein comprises or consists of a protein having SEQ ID NO: 67. In some embodiments, the recombinant protein comprises or consists of a protein having SEQ ID NO: 68. In some embodiments, the recombinant protein comprises or consists of a protein having SEQ ID NO: 71.

In some embodiments, a nucleic acid is provided which encodes a recombinant protein as provided herein. In some embodiments, the nucleic acid comprises a vector. In some embodiments, the vector comprises a host cell.

Some embodiments are drawn to a method of making a recombinant protein comprising culturing the host cell with one of the nucleic acids provided herein and collecting the recombinant protein. In some embodiments, the host cell is a eukaryotic host cell. In some embodiments, the host cell is a mammalian host cell. In some embodiments, the mammalian host is a CHO cell. In some embodiments, the recombinant protein is collected using an alpha-1-antitrypsin-Fc capture select media at neutral pH.

Some embodiments are drawn to a method of treating an alpha-1-antitrypsin deficiency in a patient in need thereof comprising administering a recombinant protein, as provided herein, to the patient. In some embodiments, the recombinant protein comprises a pharmaceutical composition. In some embodiments, administration of the recombinant protein is administered about once a week or at longer intervals than about once a week, about once every 10 days or at longer intervals than about once every ten days, about once every 15 days or at longer intervals than about once every 15 days, about once every 20 days or at longer intervals than about once every 20 days, about once every 25 days or at longer intervals than about once every 25 days, about once every month or at longer intervals than about once every month, or about once every two months or at longer intervals than about once every two months.

Some embodiments are drawn to a method of improving the pharmacokinetics of a protein comprising an alpha-1-antitrypsin serpin domain. In some embodiments, the protein is any of the recombinant proteins set forth herein. In some embodiments improved pharmacokinetics means an improvement with respect to Prolastin.

### BRIEF DESCRIPTION OF THE DRAWINGS

FIGS. 1A and 1B depict the structure of the alpha-1-antitrypsin protein and gene. The top, FIG. 1A, shows the normal protein in its linear form, a 394 amino acid single chain protein with three asparaginyl-linked complex carbohydrates side chains at residues 46, 83, and 247 according to SEQ ID NO: 1. The bottom, FIG. 1B, shows the gene (SEQ ID NO: 69) and consists of three non-coding exons (IA, IB, and IC) and four coding exons (II-V). Alpha helices are shown with boxes and beta sheets are shown with squiggles. The start codon is in exon II followed by a 24 residue signal peptide. The carbohydrate attachment sites are shown in the figure as CHO.

FIG. 2 shows locations of mutations associated with alpha-1-antitrypsin gene associated with alpha-1-antitrypsin deficiency.

FIG. 3 shows alpha-1-antitrypsin secondary structure.

FIG. 4A depicts purified CE-SDS for PP11288. FIG. 4B depicts purified CE-SDS for PP11289. FIG. 4C depicts purified CE-SDS for PP11290. FIG. 4D depicts purified CE-SDS for PP11291. FIG. 4E depicts purified CE-SDS for PP11292. FIG. 4F depicts purified CE-SDS for purified samples (reduced overlay.) FIG. 4G depicts purified CE-SDS for purified samples (non-reducing conditions.)

FIG. 5 depicts the stability analysis of 5 AAT-Albumin fusions.

FIG. 6 depicts results for an hNE inhibition assay with 5 AAT-albumin fusions.

FIG. 7A depicts purified CE-SDS for PP13579. FIG. 7B depicts purified CE-SDS for PP13580. FIG. 7C depicts purified CE-SDS for PP13581. FIG. 7D depicts purified CE-SDS for PP13582. FIG. 7E depicts purified CE-SDS for PP13583. FIG. 7F depicts purified CE-SDS for PP13584. FIG. 7G depicts purified CE-SDS for PP13585. FIG. 7H depicts purified CE-SDS for PP13586. FIG. 7I depicts purified CE-SDS for PP13587. FIG. 7J depicts purified CE-SDS for PP13588. FIG. 7K depicts purified CE-SDS for PP13589. FIG. 7L depicts purified CE-SDS for PP13590. FIG. 7M depicts purified CE-SDS for PP13592. FIG. 7N depicts purified CE-SDS for PP 13593. FIG. 7O depicts purified CE-SDS for PP13594. FIG. 7P depicts purified CE-SDS for PP13595. FIG. 7Q depicts purified CE-SDS for PP13596. FIG. 7R depicts purified CE-SDS for PP13597. FIG. 7S depicts purified CE-SDS for PP13598. FIG. 7T depicts purified CE-SDS for PP13599. FIG. 7U depicts purified CE SDS for PP13600.

FIG. 8 depicts the stability analysis of 21 AAT-Albumin fusions.

FIG. 9A depicts IC50 analysis of AAT mutants. FIG. 9B depicts IC50 analysis of AAT mutants. FIG. 9C depicts IC50 analysis of AAT mutants.

FIG. 10 depicts EpiMatrix protein scores normalized and plotted on a standardized scale.

FIG. 11 depicts stability analysis of AAT variant (K335A, S283C/P361C) on base (C232S, M351V, M351L).

FIG. 12 depicts cyno PK data.

FIG. 13 depicts that AAT-HSA and prolastin after PK in monkey after 10 mg/kg was 3 compartmental.

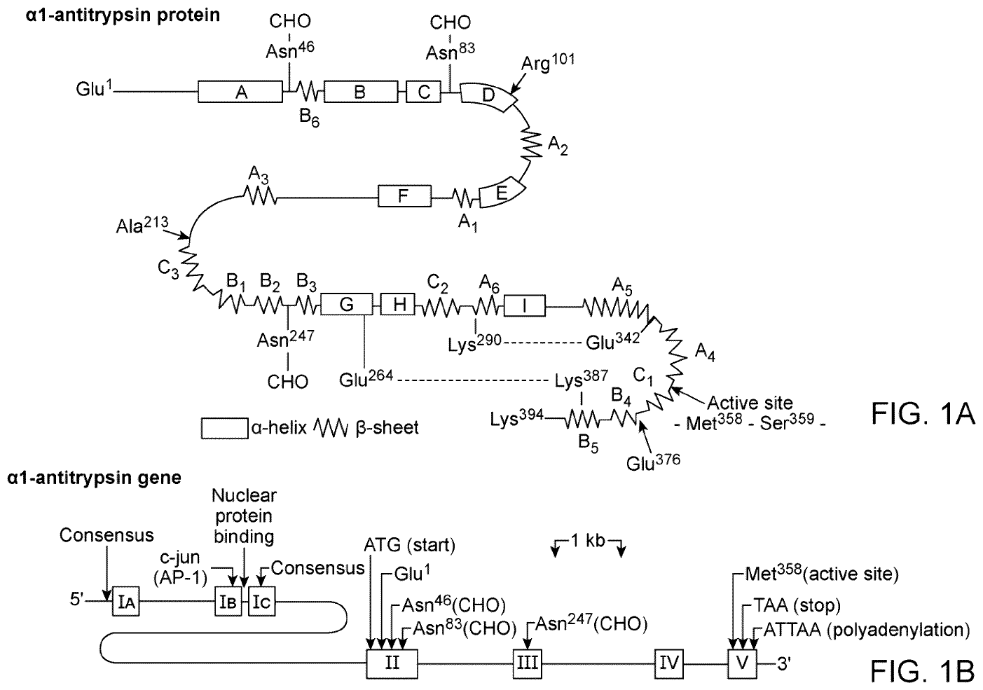

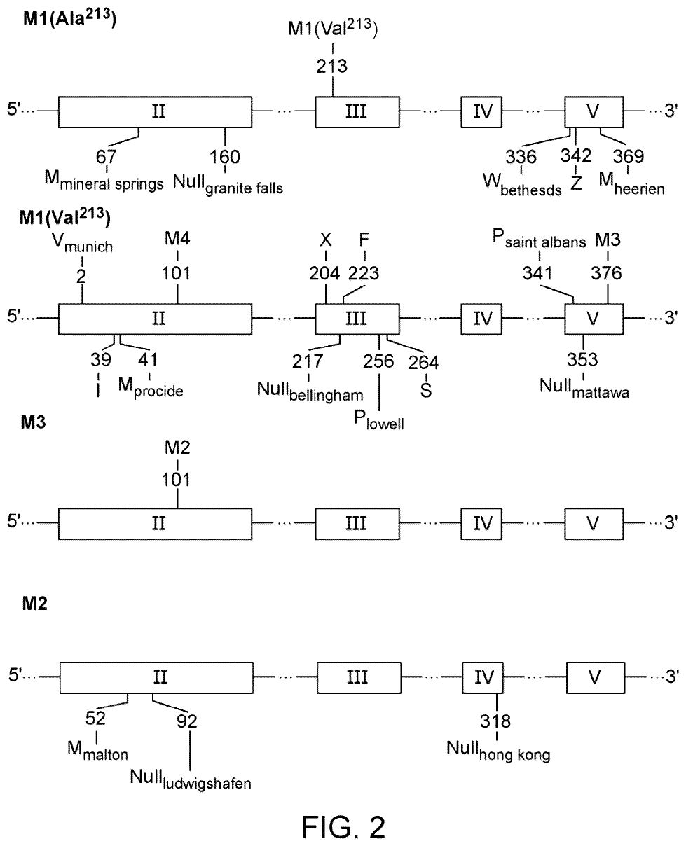

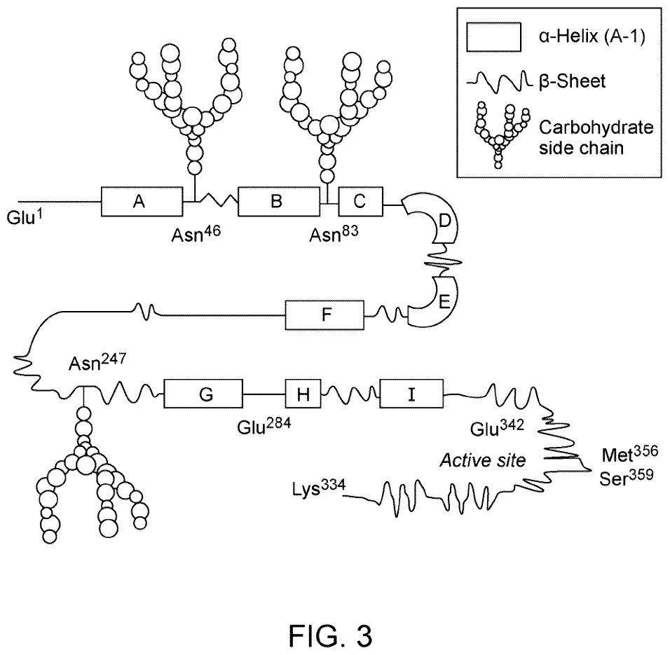

### DETAILED DESCRIPTION OF THE INVENTION

### Terminology

As used herein, the term “about” shall refer to an approximate mathematical quantity and can be used with respect to integer values as well as percentages. When an upper or lower bound limit comprises an integer value, the term about shall refer to a boundary that is approximate and the term shall also include everything above that boundary (with respect to a lower boundary) or below that boundary (with respect to an upper boundary.) For example, a portion of a sequence that comprises “about” 20 residues of a 393 residue sequence shall include a boundary that is approximately 20 residues and all integer values above that value.

As used herein, “albumin” shall refer to an albumin protein or albumin fragment, portion, or variant having one or more functional activities of albumin or to a nucleic acid sequence encoding an albumin protein, fragment, portion, or variant thereof having one or more functional activities of albumin. Albumin may be derived from any vertebrate, especially any mammal. In some embodiments, the albumin is derived from human, cow, sheep, or pig. Non-mammalian albumins include, but are not limited to, hen and salmon. In some embodiments, an albumin derived from any vertebrate may be substituted for human albumin or human serum albumin.

The term “antibody” as used herein refers to a type of immunoglobulin molecule and is used in its broadest sense. An antibody specifically includes intact antibodies (e.g., intact immunoglobulins), and antibody fragments. Antibodies comprise at least one antigen-binding domain. One example of an antigen-binding domain is an antigen binding domain formed by a V_{H}-V_{L} dimer. Other examples of antibodies are set forth in the following sections.

The V_{H} and V_{L} regions may be further subdivided into regions of hypervariability (“hypervariable regions (HVRs);” also called CDRs, as defined herein) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and confer antigen specificity and binding affinity to the antibody. See Kabat et al., wSequences of Proteins of Immunological Interest 5th ed. (1991) Public Health Service, National Institutes of Health, Bethesda, MD, incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

An “antibody fragment” comprises a portion of an intact antibody, such as the antigen binding or variable region of an intact antibody. Antibody fragments include, for example, Fv fragments, Fab fragments, F(ab′)2 fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (CH₁) of the heavy chain. Fab fragments may be generated, for example, by papain digestion of a full length antibody.

“F(ab′)2” fragments contain two Fab′ fragments joined, near the hinge region, by disulfide bonds. F(ab′)2 fragments may be generated, for example, by pepsin digestion of an intact antibody. The F(ab′) fragments can be dissociated, for example, by treatment with ß-mercaptoethanol.

“Single-chain Fv” or “sFv” or “scFv” antibody fragments comprise a V_{H} domain and a V_{L} domain in a single polypeptide chain linked by a peptide linker. See Pluckthun A. (1994.) “scFv-Fc” fragments comprise an scFv attached to an Fc domain. For example, an Fc domain may be attached to the C-terminal of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody).

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

As used herein, a “single domain antibody” shall refer to an antibody whose CDRs are part of a single domain polypeptide. Examples include, but are not limited to, heavy chain antibodies, antibodies naturally devoid of light chains, single domain antibodies derived from conventional four chain antibodies and single domain scaffolds other than those derived from antibodies. Single domain antibodies may be derived from any species including, but not limited to, mouse, human, camel, llama, goat, rabbit, shark, and bovine. As an example, a single domain antibody may be raised in Camelidae species such as camel, dromedary, alpaca, and guanaco. In some embodiment, a single domain antibody comprises a sequence set forth in any one of SEQ ID NOs: 22-28 and SEQ ID NOs: 29-36.

As used herein, “complementary-determining regions” or “CDRs” are part of the variable chains in antibodies. The CDRs are responsible for specific antibodies. One skilled in the art would appreciate how to determine the location of CDRs within an antibody for specific antibodies and there are several systems determining CDR location. For the sake of clarity, in this application, CDRs for single domain antibodies shall be determined according to US 2010/011339, which is incorporated by reference in its entirety herein.

The term “epitope” means a portion of an antigen capable of specific binding to a binding protein, such as, for example, without limitation, an antibody. Epitopes frequently consist of surface-accessible amino acid residues and/or sugar side chains and may have specific three-dimensional structural characteristics, as well as specific charge characteristics. An epitope may comprise amino acid residues that are directly involved in the binding, and other amino acid residues, which are not directly involved in the binding. The epitope to which an antibody binds can be determined using known techniques for epitope determination.

As used herein, “human albumin” refers to a human albumin protein or human albumin fragment or variant or portion having one or more functional activities of human albumin or nucleic acid sequence encoding a human albumin, fragment, portion, or variant thereof having one or more functional activities of human albumin. Human albumin is a polypeptide sequence of 609 amino acids, the first 18 amino acids of which constitute the leader sequence not found in the final blood bourne product. A human albumin is set forth in SEQ ID NO: 3 in some embodiments.

As used herein, “human serum albumin” shall refer to the human serum albumin found in human blood, which is normally devoid of the initial 18 amino acid leader sequence. In some embodiments, human serum albumin has the sequence set forth in SEQ ID NO: 5. A human serum albumin domain shall also refer to any human serum albumin protein or human serum albumin fragment or variant or portion having one or more functional activities of human serum albumin.

As used herein, “human serum albumin binding protein” or “human albumin binding domain” shall refer to any protein or protein fragment or variant having one or more binding activities to human albumin or human serum albumin.

As used herein, “alpha-1-antitrypsin” or “ai-antitrypsin” (also “A1AT,” “A1A,” or “AAT”) shall refer to an alpha-1-antitrypsin protein or an alpha-1-antitrypsin fragment or variant having one or more functional activities of alpha-1-antitrypsin or a nucleic acid sequence encoding an alpha-1-antitrypsin protein, fragment, or variant thereof having one or more functional activities of alpha-1-antitrypsin. In some embodiments, the alpha-1-antitrypsin protein has the sequence set forth in SEQ ID NO: 1.

As used herein, “alpha-1-antitrypsin serpin domain” or “ai-antitrypsin serpin domain” (also “A1AT, domain” “A1A domain,” or “AAT domain”) shall refer to an alpha-1-antitrypsin serpin domain or an alpha-1-antitrypsin serpin domain fragment or variant having one or more functional activities of an alpha-1-antitrypsin protein or fragment or a nucleic acid sequence encoding an alpha-1-antitrypsin protein serpin domain or fragment, or variant thereof having one or more functional activities of alpha-1-antitrypsin protein.

As used herein, “alpha-1-antitrypsin deficiency” or “AATD” is a genetic disorder that causes defective production of alpha-1-antitrypsin. The disease leads to a decrease of alpha-1-antitrypsin and is also referred to as hereditary pulmonary emphysema and results in lung and liver disease. There are several forms and degrees of the deficiency. The form and degree depend on whether the sufferer has one or two copies of a defective allele. Severe alpha-1-antitrypsin deficiency causes panacinar emphysema or COPD in adult life in many people with the condition, especially if they are exposed to cigarette smoke. The disorder can also lead to various liver diseases in a minority of children and adults, and occasionally more unusual problems. Alpha-1-antitrypsin deficiency usually produces some degree of disability and reduces life expectancy.

An “effective amount” of a recombinant protein described herein or functional fragment or variant thereof refers to the amount of the polypeptide or functional fragment or variant thereof, when administered in an aggregate of multiple doses, or as part of any other type of defined treatment regimen, produces a measureable statistical improvement in outcome or prophylaxis, as evidenced by at least one clinical parameter associated with the complication.

As used herein, “neutrophil elastase” shall refer to a serine protease that destroys elastase, the rubberlike macromolecule that provides elastic recoil to the lung. When the blood contains inadequate amounts of AAT, neutrophil elastase can break down elastin, degrading the elasticity of the lungs, which results in respiratory complications, such as chronic obstructive pulmonary disease.

The term “portion” as used herein shall mean a part of a whole or piece of something such as a protein or polypeptide.

As used herein, “recombinant” in reference to a protein or polypeptide molecule, refers to a protein or polypeptide molecule expressed utilizing isolated nucleic acid molecules or recombinant nucleic acid molecules.

The term “variant” or “variants” as used herein shall mean a different sequence of a protein including, for example, without limitation, insertions, deletions, and substitutions, either conservative or non-conservative, where such changes do not substantially alter one or more of the oncotic, useful ligand-binding, and non-immunogenic properties of an albumin or albumin binding domain or do not alter the serpin domain.

Variants of polypeptides described herein include polypeptides having at least about 75%, at least about 80%, at least about 85%, at least about 90%, at least about 95%, at least about 96%, at least about 97%, at least about 98% or at least about 99% identity with the amino acid sequence of the human wild type polypeptides provided herein. For example, variants of alpha-1-antitrypsin include polypeptides having at least about 75%, at least about 80%, at least about 85%, at least about 90%, at least about 95%, at least about 96%, at least about 97%, at least about 98%, or at least about 99% identity with the amino acid sequence of SEQ ID NO: 1. For example, variants of human albumin include polypeptides having at least about 75%, at least about 80%, at least about 85%, at least about 90%, at least about 95%, at least about 96%, at least about 97%, at least about 98%, or at least about 99% identity with the amino acid sequence of SEQ ID NO: 3 or SEQ ID NO: 5. Calculations of “identity” or “sequence homology” between two sequences (the terms are used interchangeably herein) are performed by aligning for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences. Variants include, but are not limited to, polypeptides that have been either chemically modified relative to the human wild type polypeptide and/or contain one or more amino acid sequence alterations relative to the human wild type polypeptide.

Variants of polypeptides described herein can be those having amino acid modifications (e.g., deletions, additions, or substitutions, such as conservative substations) from the wild type amino acid sequence of the polypeptide. In some embodiments, a variant of AAT can differ by at least 1, 2, 3, 4, 5, or more resides from alpha-1-antitrypsin (SEQ ID NO: 1) and a variant of a human serum albumin can vary by at least 1, 2, 3, 4, or 5 residues from human serum albumin (SEQ ID NO: 5).

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Recombinant proteins having conservative substitutions are intended to be within the scope of the current invention.

The term “fusion” or “fusion molecule” can refer to a polypeptide or a nucleic acid fusion, depending on the context. It may include a full-length sequence or a protein or nucleic acid or a fragment thereof. It may also include a recombinant protein with or without a linker molecule.

An “isolated” protein refers to a protein that is removed from at least about 90% of at least one component of a natural sample from which the isolated protein can be obtained. Proteins can be “of at least about” a certain degree of purity if the species or population of species of interest is at least about 5, 25, 50, 75, 80, 90, 92, 95, 98, or 99% pure on a weight-weight basis.

As used herein, the term “oncotic” shall refer to oncotic pressure, or colloid osmotic pressure. It is a form of osmotic pressure exerted by proteins, notably albumin, in a blood vessel's plasma that pulls water into the circulatory system. It is the opposing force to hydrostatic pressure. It has a major effect on the pressure across the glomerular filter.

A “patient,” “subject,” or “host” (these terms are used interchangeably) to be treated by the subject method may mean either a human or non-human animal. Preferably, a patient, subject, or host refers to a human patient.

The term “prophylactically treating” a disease in a subject refers to subjecting the subject to a pharmaceutical treatment, e.g., the administration of a drug, such that at least one symptom of the disease is prophylactically treated, that is, administered prior to clinical manifestation of the unwanted condition so that it protects the host against developing the unwanted condition. “Prophylactially treating” a disease may also be referred to as “prophylaxis.” In some embodiments, prophylactic treatment prevents the disorder.

As used herein, “treating” a disorder associated with an alpha-1-antitrypsin deficiency in a subject in need or “treating” a subject having a disorder associated with an alpha-1-antitrypsin deficiency, refers to subjecting the subject to a pharmaceutical treatment, e.g., the administration of a drug, such that at least one symptom of the disease is cured, alleviated, or decreased.

Any of the treatments described herein can be administered in combination with another agent or therapy. The term “combination” refers to the use of the two or more agents or therapies to treat the same patient, wherein the use or action of the agents or therapies overlap in time. The agents or therapies can be administered at the same time (e.g., as a single formulation that is administered to a patient or as two separate formulations administered concurrently) or sequentially in any order.

### Recombinant Protein

Some embodiments provide a recombinant protein comprising an alpha-1-antitrypsin serpin domain, or functional fragment or variant thereof, and a human serum albumin domain or functional fragment or variant thereof, or human serum albumin binding domain or functional fragment or variant thereof.

### Alpha-1-Antitrypsin Serpin Domain

In some embodiments, the alpha-1-antitrypsin serpin domain comprises a full length alpha-1-antitrypsin. In some embodiments, the alpha-1-antitrypsin is a protein that comprises the sequence set forth in SEQ ID NO: 1.

In some embodiments, the alpha-1-antitrypsin serpin domain comprises one or more mutations. In some embodiments, the one or more mutations comprises a mutation in at least one of the following residue positions according to SEQ ID NO 1: 51, 100, 114, 117, 163, 164, 165, 168, 169, 172, 173, 174, 183, 189, 232, 283, 300, 302, 303, 304, 306, 330, 331, 333, 335, 336, 337, 338, 339, 340, 351, 356, 358, or 361. In some embodiments, the one or more mutations comprises one or more of the following mutations according to SEQ ID NO F51L, L100F, L100C, Ti 14F, G117F, K163T, G164V, T165S, K168C, K168I, K168A, K169V, L172V, L172A, L172C, V173C, K174T, A183V, F189C, F189I, F189V, C232S, S283C, K300A, V302A, L303A, G304A, L306A, S330R, K331F, K331I, K331V, K331T, K331C, V333C, V333A, K335A, K331F, K335G, K335T, A336G, V337C, V337A, L338A, T339S, I340V, M351V, I356M, M358I, M358P, M358A, M358R, M358L, M358V, or P361C. In some embodiments, the one or more mutations comprises F51L, G117F, K168C, F189C, C232S, S283C, K331F, K335A, M351V, M358V, M358L, or P361C according to SEQ ID NO: 1. In some embodiments, the one or more mutations are both of K168C and F189C according to SEQ ID NO: 1. In some embodiments, the one or more mutations are both of P361C and S283C according to SEQ ID NO: 1.

In some embodiments, the alpha-1-antitrypsin serpin domain has one or more mutations comprising C232S according to SEQ ID NO: 1. In some embodiments, the alpha-1-antitrypsin serpin domain has one or more mutations comprising M351V and M358V according to SEQ ID NO: 1. In some embodiments, the alpha-1-antitrypsin serpin domain has one or more mutations comprising M351V and M358L according to SEQ ID NO: 1. In some embodiments, the alpha-1-antitrypsin serpin domain has one or more mutations comprising C232S, M351V, and M358V according to SEQ ID NO: 1.

In some embodiments, the alpha-1-antitrypsin serpin domain has one or more mutations comprising F51L, C232S, M351V and M358L according to SEQ ID NO: 1. In some embodiments, the alpha-1-antitrypsin serpin domain has one or more mutations comprising G117F, C232S, M351V and M358L according to SEQ ID NO: 1. In some embodiments, the alpha-1-antitrypsin serpin domain has one or more mutations comprising C232S, K331F, M351V and M358L according to SEQ ID NO: 1. In some embodiments, the alpha-1-antitrypsin serpin domain has one or more mutations comprising C232S, K335A, M351V and M358L according to SEQ ID NO: 1. In some embodiments, the alpha-1-antitrypsin serpin domain has one or more mutations comprising K168C, F189C, C232S, M351V, and M358L according to SEQ ID NO: 1. In some embodiments, the alpha-1-antitrypsin serpin domain has one or more mutations comprising C232S, S283C, M351V, M358L, and P361C according to SEQ ID NO: 1. In some embodiments, the alpha-1-antitrypsin serpin domain has one or more mutations comprising F51L, G117F, C232S, M351V, and M358L according to SEQ ID NO: 1. In some embodiments, the alpha-1-antitrypsin serpin domain has one or more mutations comprising F51L, C232S, K331F, M351V, and M358L according to SEQ ID NO: 1. In some embodiments, the alpha-1-antitrypsin serpin domain has one or more mutations comprising F51L, C232S, K335A, M351V, and M358L according to SEQ ID NO: 1. In some embodiments, the alpha-1-antitrypsin serpin domain has one or more mutations comprising K51L, K168C, F189C, C232S, M351V, and M358L according to SEQ ID NO: 1. In some embodiments, the alpha-1-antitrypsin serpin domain has one or more mutations comprising F51L, C232S, S283C, M351V, M358L, and P361C according to SEQ ID NO: 1. In some embodiments, the alpha-1-antitrypsin serpin domain has one or more mutations comprising G117F, C232S, K331F, M351V, and M358L according to SEQ ID NO: 1. In some embodiments, the alpha-1-antitrypsin serpin domain has one or more mutations comprising G117F, K335A, C232S, M351V, and M358L according to SEQ ID NO: 1. In some embodiments, the alpha-1-antitrypsin serpin domain has one or more mutations comprising G117F, K168C, F189C, C232S, M351V, and M358L according to SEQ ID NO: 1. In some embodiments, the alpha-1-antitrypsin serpin domain has one or more mutations comprising G117F, C232S, S283C, M351V, M358L, and P361C according to SEQ ID NO: 1. In some embodiments, the alpha-1-antitrypsin serpin domain has one or more mutations comprising C232S, K331F, K335A, M351V, and M358L according to SEQ ID NO: 1. In some embodiments, the alpha-1-antitrypsin serpin domain has one or more mutations comprising K168C, F189C, C232S, K331F, M351V, and M358L according to SEQ ID NO: 1. In some embodiments, the alpha-1-antitrypsin serpin domain has one or more mutations comprising C232S, S283C, K331F, M351V, M358L, and P361C according to SEQ ID NO: 1. In some embodiments, the alpha-1-antitrypsin serpin domain has one or more mutations comprising K168C, F189C, C232S, K335A, M351V, and M358L according to SEQ ID NO: 1. In some embodiments, the alpha-1-antitrypsin serpin domain has one or more mutations comprising C232S, S283C, K335A, M351V, M358L, and P361C according to SEQ ID NO: 1. In some embodiments, the alpha-1-antitrypsin serpin domain has one or more mutations comprising K168C, F189C, C232S, S283C, M351V, M358L, and P361C according to SEQ ID NO: 1. In some embodiments, the alpha-1-antitrypsin serpin domain comprises a point mutation K335A according to SEQ ID NO: 1. In some embodiments, the alpha-1-antitrypsin serpin domain comprises a point mutation K331F according to SEQ ID NO: 1. In some embodiments, the alpha-1-antitrypsin serpin domain comprises a point mutation G117F according to SEQ ID NO: 1. In some embodiments, the alpha-1-antitrypsin serpin domain comprises a point mutation S283C according to SEQ ID NO: 1. In some embodiments, the alpha-1-antitrypsin serpin domain comprises a point mutation P361C according to SEQ ID NO: 1.

In some embodiments, glycan sites in an alpha-1-antitrypsin serpin domain are truncated and deleted. In some embodiments, the first residue of SEQ ID NO: 1 is mutated so that the glycan sites are deleted. In some embodiments, residues 1-46 of SEQ ID NO: 1 are deleted.

In some embodiments, the one or more mutations provide improved stability including improved stability to oxidation, retention of potency against human neutrophil elastase, resistance to polymerization, and ease of production, wherein the improved stability is measured against wild type.

In some embodiments, the one or more mutations are one or more mutations as set forth in Table 1 and in Tables 2-5 with residue positions being set forth according to SEQ ID NO: 1.

TABLE 1
AAT-Albumin Fusion

| | |
|---|---|
| Base | WT |
| Base | C232S |
| Base | M35IV, M358V |
| Base | M35IV, M358L |
| Base | C2325, M351V, M358L |

AAT Mutants

| | | | | | | K168C/ | P361C- |
|---|---|---|---|---|---|---|---|
| | Base | F511 | G117F | K331F | K335A | F189C | S283C |
| Base | | 1 | 2 | 3 | 4 | 5 | 6 |
| FS1L | | | 7 | 8 | 9 | 10 | 11 |
| G117F | | | | 12 | 13 | 14 | 15 |
| K331F | | | | | 16 | 17 | 18 |
| K335A | | | | | | 19 | 20 |
| K168C/ | | | | | | | 21 |
| F189C | | | | | | | |
| P361C- | | | | | | | |
| S283C | | | | | | | |

In some embodiments, the alpha-1-antitrypsin serpin domain comprises a portion of the alpha-1-antitrypsin. In some embodiments, the portion of the alpha-1-antitrypsin serpin domain has an activity that is equal to or greater than a full length human alpha-1-antitrypsin such as, for example, without limitation the alpha-1-antitrypsin serpin domain having the sequence set forth in SEQ ID NO: 1. In some embodiments, the portion is at least about 20 residues of an alpha-1-antitrypsin, at least about 30 residues of an alpha-1-antitrypsin, at least about 40 residues of an alpha-1-antitrypsin, at least about 50 residues of an alpha-1-antitrypsin, at least about 60 residues of an alpha-1-antitrypsin, at least about 70 residues of an alpha-1-antitrypsin, at least about 80 residues of an alpha-1-antitrypsin, at least about 90 residues of an alpha-1-antitrypsin, at least about 100 residues of an alpha-1-antitrypsin, at least about 110 residues of an alpha-1-antitrypsin, at least about 120 residues of an alpha-1-antitrypsin, at least about 130 residues of an alpha-1-antitrypsin, at least about 140 residues of an alpha-1-antitrypsin, at least about 150 residues of an alpha-1-antitrypsin, at least about 160 residues of an alpha-1-antitrypsin, at least about 170 residues of an alpha-1-antitrypsin, at least about 180 residues of an alpha-1-antitrypsin, at least about 190 residues of an alpha-1 antitrypsin, at least about 200 residues of an alpha-1-antitrypsin, at least about 210 residues of an alpha-1-antitrypsin, at least about 220 residues of an alpha-1-antitrypsin, at least about 230 residues of an alpha-1-antitrypsin, at least about 240 residues of an alpha-1-antitrypsin, at least about 250 residues of an alpha-1-antitrypsin, at least about 260 residues of an alpha-1-antitrypsin, at least about 270 residues of an alpha-1-antitrypsin, at least about 280 residues of an alpha-1-antitrypsin, at least about 290 residues of an alpha-1-antitrypsin, at least about 300 residues of an alpha-1-antitrypsin, at least about 310 residues of art alpha-1-antitrypsin, at least about 320 residues of an alpha-1-antitrypsin, at least about 330 residues of an alpha-1-antitrypsin, at least about 340 residues of an alpha-1-antitrypsin, at least about 350 residues of an alpha-1-antitrypsin, at least about 360 residues of an alpha-1-antitrypsin, at least about 370 residues of an alpha-1-antitrypsin, at least about 380 residues of an alpha-1-antitrypsin, or at least about 390 residues of an alpha-1-antitrypsin.

In some embodiments, the portion is at least about 20 residues of an alpha-1-antitrypsin having the sequence set forth in SEQ ID NO: 1, at least about 30 residues of an alpha-1-antitrypsin having the sequence set forth in SEQ ID NO: 1, at least about 40 residues of an alpha-1-antitrypsin having the sequence set forth in SEQ ID NO: 1, at least about 50 residues of an alpha-1-antitrypsin having the sequence set forth in SEQ. ID NO: 1, at least about 60 residues of an alpha-1-antitrypsin having the sequence set forth in SEQ ID NO: 1, at least about 70 residues of an alpha-1-antitrypsin having the sequence set forth in SEQ ID NO: 1, at least about 80 residues of an alpha-1-antitrypsin having the sequence set forth in SEQ ID NO: 1, at least about 90 residues of an alpha-1-antitrypsin having the sequence set forth in SEQ ID NO; 1, at least about 100 residues of an alpha-1-antitrypsin having the sequence set forth in SEQ ID NO: 1, at least about 110 residues of an alpha-1-antitrypsin having the sequence set forth in SEQ ID NO: 1, at least about 120 residues of an alpha-1-antitrypsin having the sequence set forth in SEQ ID NO: 1, at least about 130 residues of an alpha-1-antitrypsin having the sequence set forth in SEQ ID NO: 1, at least about 140 residues of an alpha-1-antitrypsin having the sequence set forth in SEQ ID NO: 1, at least about 150 residues of an alpha-1-antitrypsin having the sequence set forth in SEQ ID NO: 1, at least about 160 residues of an alpha-1-antitrypsin having the sequence set forth in SEQ ID NO: 1, at least about 170 residues of an alpha-1-antitrypsin having the sequence set forth in SEQ ID NO: 1, at least about 180 residues of an alpha-1-antitrypsin having the sequence set forth in SEQ ID NO: 1, at least about 190 residues of an alpha-1-antitrypsin having the sequence set forth in SEQ ID NO: 1, at least about 200 residues of an alpha-1-antitrypsin having the sequence set forth in SEQ ID NO: 1, at least about 210 residues of an alpha-1-antitrypsin having the sequence set forth in SEQ ID NO: 1, at least about 220 residues of an alpha-1-antitrypsin having the sequence set forth in SEQ ID NO: 1, at least about 230 residues of an alpha-1-antitrypsin having the sequence set forth in SEQ ID NO: 1, at least about 240 residues of an alpha-1-antitrypsin having the sequence set forth in SEQ ID NO: 1, at least about 250 residues of an alpha-1-antitrypsin having the sequence set forth in SEQ ID NO: 1, at least about 260 residues of an alpha-1-antitrypsin having the sequence set forth in SEQ ID NO: 1, at least about 270 residues of an alpha-1-antitrypsin having the sequence set forth in SEQ ID NO: 1, at least about 280 residues of an alpha-1-antitrypsin having the sequence set forth in SEQ ID NO: 1, at least about 290 residues of an alpha-1-antitrypsin having the sequence set forth in SEQ ID NO: 1, at least about 300 residues of an alpha-1-antitrypsin having the sequence set forth in SEQ ID NO: 1, at least about residues of an alpha-1-antitrypsin having the sequence set forth in SEQ ID NO: 1, at least about 320 residues of an alpha-1-antitrypsin having the sequence set forth in SEQ ID NO: 1, at least about 330 residues of an alpha-1-antitrypsin having the sequence set forth in SEQ ID NO: 1, at least about 340 residues of an alpha-1-antitrypsin having the sequence set forth in SEQ ID NO: 1, at least about 350 residues of an alpha-1-antitrypsin having the sequence set forth in SEQ ID NO: 1, at least about 360 residues of an alpha-1-antitrypsin having the sequence set forth in SEQ ID NO: 1, at least about 370 residues of an alpha-1-antitrypsin having the sequence set forth in SEQ ID NO: 1, at least about 380 residues of an alpha-1-antitrypsin having the sequence set forth in SEQ ID NO: 1, or at least about 390 residues of an alpha-1-antitrypsin having the sequence set forth in SEQ. ID NO: 1.

In some embodiments, an alpha-1-antitrypsin serpin domain comprises a variant of a normal alpha-1-antitrypsin as set forth in SEQ ID NO: 1. In some embodiments, the variant comprises an active portion of the alpha-1-antitrypsin serpin domain.

### Albumin

In some embodiments, the human serum albumin domain comprises a full-length human albumin. In some embodiments, the human serum albumin domain comprises the sequence set forth in SEQ ID NO: 3 or SEQ ID NO: 5. In some embodiments, the human serum albumin domain has a sequence comprising SEQ ID NO: 3 or SEQ NO: 5.

In some embodiments, the human senate albumin domain comprises one or more mutations. In some embodiments, the one or more mutations comprises mutations in one or more of the following locations: 407, 408, 409, 410, 413, and 414, wherein the location numbers are with respect to SEQ ID NO. 5. In some embodiments, the one or more mutations comprises at least one of the following mutations: L407A, L408V, V409A, R410A, L413Q, and L414Q, wherein the position numbers are with respect to SEQ ID NO. 5 (See, e.g., International Publication No. WO95/23857, hereby incorporated in its entirety by reference herein.) in some embodiments, the one or more mutations comprises L407A, L408V, V409A, and R410A, wherein the position numbers are with respect to SEQ ID NO. 5. In some embodiments, the one or more mutations comprises R410A, L413Q, and L414Q, wherein the position numbers are with respect to SEQ ID NO. 5. In some embodiments, the one or more mutations improves stability and/or increases production of the recombinant protein.

In some embodiments, the human serum albumin domain comprises a portion of human albumin. In some embodiments, the portion is at least about 20 residues of a human serum albumin, at least about 30 residues of a human serum albumin, at least about 40 residues of a human serum albumin, at least about 50 residues of a human serum albumin, at least about 60 residues of a human serum albumin, at least about 70 residues of a human serum albumin, at least about 80 residues of a human serum albumin, at least about 90 residues of a human serum albumin, at least about 100 residues of a human serum albumin, at least about 110 residues of a human serum albumin, at least about 120 residues of a human serum albumin, at least about 130 residues of a human serum albumin, at least about 140 residues of a human serum albumin, at least about 150 residues of a human serum albumin, at least about 160 residues of a human serum albumin, at least about 170 residues of a human serum albumin, at least about 180 residues of a human serum albumin, at least about 190 residues of a human serum albumin, at least about 200 residues of a human serum albumin, at least about 210 residues of a human serum albumin, at least about 220 residues of a human. serum albumin, at least about 230 residues of a human serum albumin, at least about 240 residues of a human serum albumin, at least about 250 residues of a human. serum albumin, at least about 260 residues of a human serum albumin, at least about 270 residues of a human serum albumin, at least about 280 residues of a human serum albumin, at least about 290 residues of a human serum albumin, at least about 300 residues of a human serum albumin, at least about 310 residues of a human serum albumin, at least about 320 residues of a human serum albumin, at least about 330 residues of a human serum albumin, at least about 340 residues of a human serum albumin, at least about 350 residues of a human serum albumin, at least about 360 residues of a human serum albumin, at least about 370 residues of a human. serum albumin, at least about 380 residues of a human serum albumin, at least about 390 residues of a human serum albumin, at least about 400 residues of a human serum albumin, at least about 410 residues of a human serum albumin, at least about 420 residues of a human serum albumin, at least about 430 residues of a human serum albumin, at least about 440 residues of a human serum albumin, at least about 450 residues of a human serum albumin, at least about 460 residues of a human serum albumin, at least about 470 residues of a human serum albumin, at least about 480 residues of a human serum albumin, at least about 490 residues of a human serum albumin, at least about 500 residues of a human serum albumin, at least about 510 residues of a human serum albumin. at least about 520 residues of a human. serum albumin, at least about 530 residues of a human serum albumin, at least about 540 residues of a human serum albumin, at least about 550 residues of a human serum albumin, at least about 560 residues of a human serum albumin, at least about 570 residues of a human serum albumin, at least about 580 residues of a human serum albumin, at least about 590 residues of a human serum albumin, or at least about 600 residues of a human serum albumin.

In some embodiments, the portion is at least about 20 residues of a human serum albumin having the sequence set forth in SEQ ID NO: 3 or SEQ ID NO: 5, at least about 30 residues of a human serum albumin having the sequence set forth in SEQ ID NO: 3 or SEQ ID NO: 5, at least about 40 residues of a human serum albumin having the sequence set forth in SEQ ID NO: 3 or SEQ ID NO: 5, at least about 50 residues of a human serum. albumin having the sequence set forth in SEQ ID NO: 3 or SEQ ID NO: 5, at least about 60 residues of a human serum albumin having the sequence set forth in SEQ ID NO: 3 or SEQ ID NO: 5, at least about 70 residues of a human serum albumin having the sequence set forth in SEQ ID NO: 3 or SEQ ID NO: 5, at least about 80 residues of a human serum albumin having the sequence set forth in SEQ ID NO: 3 or SEQ ID NO: 5, at least about 90 residues of a human serum albumin having the sequence set forth in SEQ ID NO: 3 or SEQ ID NO: 5, at least about 100 residues of a human serum albumin having the sequence set forth in SEQ ID NO: 3 or SEQ ID NO: 5, at least about 110 residues of a human serum albumin having the sequence set forth in SEQ ID NO: 3 or SEQ ID NO: 5, at least about 120 residues of a human serum albumin having the sequence set forth in SEQ ID NO: 3 or SEQ ID NO: 5, at least about 130 residues of a human serum albumin having the sequence set forth in SEQ ID NO: 3 or SEQ ID NO: 5, at least about 140 residues of a human serum albumin having the sequence set forth in SEQ ID NO: 3 or SEQ ID NO: 5, at least about 150 residues of a human serum albumin having the sequence set forth in SEQ ID NO: 3 or SEQ ID NO: 5, at least about 160 residues of a human serum albumin having the sequence set forth in SEQ ID NO: 3 or SEQ ID NO: 5, at least about 170 residues of a human serum albumin having the sequence set forth in SEQ ID NO: 3 or SEQ ID NO: 5, at least about 180 residues of a human serum albumin having the sequence set forth in SEQ ID NO: 3 or SEQ ID NO: 5, at least about 190 residues of a human serum albumin having the sequence set forth in SEQ ID NO: 3 or SEQ ID NO: 5, at least about 200 residues of a human serum albumin having the sequence set forth in SEQ ID NO: 3 or SEQ ID NO: 5, at least about 210 residues of a human serum albumin having the sequence set forth in SEQ ID NO: 3 or SEQ ID NO: 5, at least about 220 residues of a human serum albumin having the sequence set forth in SEQ ID NO: 3 or SEQ ID NO: 5, at least about 230 residues of a human serum albumin having the sequence set forth in SEQ ED NO: 3 or SEQ ED NO: 5, at least about 240 residues of a human serum albumin having the sequence set forth in SEQ ID NO: 3 or SEQ ID NO: 5, at least about 250 residues of a human serum albumin having the sequence set forth in SEQ ID NO: 3 or SEQ ID NO: 5, at least about 260 residues of a human serum albumin having the sequence set forth in SEQ ID NO: 3 or SEQ ID NO: 5, at least about 270 residues of a human serum albumin having the sequence set forth in SEQ ID NO: 3 or SEQ ID NO: 5, at least about 280 residues of a human serum albumin having the sequence set forth in SEQ ID NO: 3 or SEQ ID NO: 5, at least about 290 residues of a human serum albumin having the sequence set forth in SEQ ID NO: 3 or SEQ ID NO: 5, at least about 300 residues of a human serum albumin having the sequence set forth in SEQ ID NO: 3 or SEQ ID NO: 5, at least about 310 residues of a human serum albumin having the sequence set forth in SEQ ID NO: 3 or SEQ ID NO: 5, at least about 320 residues of a human serum albumin having the sequence set forth in SEQ ID NO: 3 or SEQ ID NO: 5, at least about 330 residues of a human serum albumin having the sequence set forth in SEQ ID NO: 3 or SEQ ID NO: 5, at least about 340 residues of a human serum albumin having the sequence set forth in SEQ ID NO: 3 or SEQ ID NO: 5, at least about 350 residues of a human serum having the sequence set forth in SEQ ID NO: 3 or SEQ ID NO: 5, at least about 360 residues of a human serum albumin having the sequence set forth in SEQ ID NO: 3 or SEQ ID NO: 5, at least about 370 residues of a human serum albumin having the sequence set forth in SEQ ID NO: 3 or SEQ ID NO: 5, at least about 380 residues of a human serum albumin having the sequence set forth in SEQ ID NO: 3 or SEQ ID NO: 5, at least about 390 residues of a human serum albumin having the sequence set forth in SEQ ID NO: 3 or SEQ ID NO: 5, at least about 400 residues of a human serum albumin having the sequence set forth in SEQ ID NO: 3 or SEQ ID NO: 5, at least about 410 residues of a human serum albumin having the sequence set forth in SEQ ID NO: 3 or SEQ ID NO: 5, at least about 420 residues of a human serum albumin having the sequence set forth in SEQ ID NO: 3 or SEQ ID NO: 5, at least about 430 residues of a human serum albumin having the sequence set forth in SEQ ID NO: 3 or SEQ ID NO: 5, at least about 440 residues of a human serum albumin having the sequence set forth in SEQ ID NO: 3 or SEQ ID NO: 5, at least about 450 residues of a human serum albumin having the sequence set forth in SEQ ID NO: 3 or SEQ ID NO: 5, at least about 460 residues of a human serum albumin having the sequence set forth in SEQ ID NO: 3 or SEQ ID NO: 5, at least about 470 residues of a human serum albumin having the sequence set forth in SEQ ID NO: 3 or SEQ ID NO: 5, at least about 480 residues of a human serum albumin having the sequence set forth in SEQ ID NO: 3 or SEQ ID NO: 5, at least about 490 residues of a human serum albumin having the sequence set forth in SEQ ID NO: 3 or SEQ ID NO: 5, at least about 500 residues of a human serum albumin having the sequence set forth in SEQ ID NO: 3 or SEQ ID NO: 5, at least about 510 residues of a human serum albumin having the sequence set forth in SEQ ID NO: 3 or SEQ ID NO: 5, at least about 520 residues of a human serum albumin having the sequence set forth in SEQ ID NO: 3 or SEQ ID NO: 5, at least about 530 residues of a human serum albumin having the sequence set forth in SEQ ID NO: 3 or SEQ ID NO: 5, at least about 540 residues of a human serum albumin having the sequence set forth in SEQ ID NO: 3 or SEQ ID NO: 5, at least about 550 residues of a human serum albumin having the sequence set forth in SEQ ID NO: 3 or SEQ ID NO: 5, at least about 560 residues of a human serum albumin having the sequence set forth in SEQ ID NO: 3 or SEQ ID NO: 5, at least about 570 residues of a human serum albumin having the sequence set forth in SEQ ID NO: 3 or SEQ ID NO: 5, at least about 580 residues of a human serum albumin having the sequence set forth in SEQ ID NO: 3 or SEQ ID NO: 5, at least about 590 residues of a human serum albumin having the sequence set forth in SEQ ID NO: 3 or SEQ ID NO: 5, or at least about 600 residues of a human serum albumin having the sequence set forth in SEQ ID NO: 3 or SEQ ID NO: 5.

In some embodiments, the portion of human serum albumin consists of or comprises at least one amino acid sequence, or a variant thereof, of the sequences set forth in amino acid residues 1-194 of SEQ ID NO: 5, amino acid residues 195-387 of SEQ ID NO: 5, amino acid residues 388-585 of SEQ ID NO: 5, 1-387 of SEQ ID NO: 5, 195-585 of SEQ ID NO: 5, or amino acid residues 1-194 of SEQ ID NO: 5 and amino acid residues 388-585 of SEQ ID NO: 5. Each domain is itself made up of two homologous subdomains namely 1-105, 120,194, 195-291, 316-387, 388-491 and 512-585, with flexible inter-subdomain linker regions (See, for example, EP 2 090 589 A1, which is incorporated by reference herein, including any drawings.) In some embodiments, the portion consists of or comprises at least one subdomain.

In some embodiments, the portion of the human serum albumin domain has an activity that is equal to or greater than a full length human albumin such as. In some embodiments, the human serum albumin has a sequence comprising the sequence set forth in SEQ ID NO: 5.

In some embodiments, the human serum albumin domain comprises a variant of a wild type human serum albumin.

### Albumin Binding Protein

In some embodiments, the human serum albumin binding domain comprises amino acid sequences that are capable of binding to human serum albumin. In some embodiments, the human serum albumin has the sequence set forth in SEQ ID NO: 3 or SEQ ID NO: 5. In some embodiments, human serum albumin comprises a portion of the human serum albumin set forth in SEQ ID NO: 5. In some embodiments, the portion of the human albumin comprises an epitope.

In some embodiments, the human serum albumin binding domain comprises an antibody. In some embodiments, the antibody is a monoclonal antibody. In some embodiments, the antibody is a humanized antibody. In some embodiments, the antibody is a human immunoglobulin ((e.g., IgG (e.g., IgG1, IgG2, IgG3, IgG4); IgE (e.g., IgE1), IgA (e.g., IgA1, IgA2), IgM (e.g., IgM1), IgD (e.g., IgD1)) or fragment, portion, or variant of a human immunoglobulin.

In some embodiments, the human serum albumin binding domain comprises a single domain antibody. In some embodiments, the single domain antibody comprises a CDR1 sequence comprising, consisting of, or consisting essentially of a sequence selected from SEQ ID NOs: 6-9. In some embodiments, the single domain antibody comprises a CDR2 sequence comprising, consisting of, or consisting essentially of a sequence selected from SEQ ID NOs: 10-16. In some embodiments, the single domain antibody comprises a CDR3 sequence comprising, consisting of, or consisting essentially of a sequence selected from SEQ ID NOs: 17-21. In some embodiments, the single domain antibody includes any one or more of the framework regions having a sequence comprising SEQ ID NOs: 37-62.

In some embodiments, the single domain antibody comprises a combination of one or more binding domains. In some embodiments, the single domain antibody comprises a combination of one or more of CDR1, CDR2, and CDR3. In some embodiments, the single domain antibody comprises a CDR1 sequence comprising, consisting of, or consisting essentially of a sequence selected from SEQ ID NOs: 6-9 and a CDR2 sequence comprising, consisting of, or consisting essentially of a sequence selected from SEQ ID NOs: 10-16. In some embodiments, the single domain antibody comprises a CDR1 sequence comprising, consisting of, or consisting essentially of a sequence selected from SEQ ID NOs: 6-9 and a CDR3 sequence comprising, consisting of, or consisting essentially of a sequence selected from SEQ ID NOs: 17-21. In some embodiments, the single domain antibody comprises a CDR2 sequence comprising, consisting of, or consisting essentially of a sequence selected from SEQ ID NOs: 10-16 and a CDR3 sequence comprising, consisting of, or consisting essentially of a sequence selected from SEQ ID NOs: 17-21. In some embodiments, the single domain antibody comprises a CDR1 sequence comprising, consisting of, or consisting essentially of a sequence selected from SEQ ID NOs: 6-9, a CDR2 sequence comprising, consisting of, or consisting essentially of a sequence selected from SEQ ID NOs: 10-16, and a CDR3 sequence comprising, consisting of, or consisting essentially of a sequence selected from SEQ ID NOs: 17-21.

In some embodiments, the single domain antibody comprises a sequence selected from SEQ ID NOs: 22-28.

In some embodiments, the single domain antibody comprises a humanized sequence selected from SEQ ID NOs: 29-36. In some embodiments, the single domain antibody comprises a humanized sequence comprising, consisting of, or consisting essentially of SEQ ID NO: 29. In some embodiments, the single domain antibody comprises a humanized sequence comprising, consisting of, or consisting essentially of SEQ ID NO: 30. In some embodiments, the single domain antibody comprises a humanized sequence comprising, consisting of, or consisting essentially of SEQ ID NO: 31. In some embodiments, the single domain antibody comprises a humanized sequence comprising, consisting of, or consisting essentially of SEQ ID NO: 32. In some embodiments, the single domain antibody comprises a humanized sequence having SEQ ID NO: 33. In some embodiments, the single domain antibody comprises a humanized sequence comprising, consisting of, or consisting essentially of SEQ ID NO: 34. In some embodiments, the single domain antibody comprises a humanized sequence comprising, consisting of, or consisting essentially of SEQ ID NO: 35. In some embodiments, the single domain antibody comprises a humanized sequence comprising, consisting of, or consisting essentially of SEQ ID NO: 36.

In some embodiments, the single domain antibody comprises a variant of a humanized sequence selected from SEQ ID NOs: 29-36. Variants described herein include polypeptides having at least about 75%, at least about 80%, at least about 85%, at least about 90%, at least about 95%, at least about 96%, at least about 97%, at least about 98% or at least about 99% identity with any sequence of the polypeptides provided herein. For example, variants of a humanized single domain antibody would have at least about 75%, at least about 80%, at least about 85%, at least about 90%, at least about 95%, at least about 96%, at least about 97%, at least about 98%, or at least about 99% identity with the amino acid sequence of any one of SEQ ID NOs: 29-36.

### Nucleic Acids

Some embodiments comprise an isolated nucleic acid molecule encoding a recombinant protein comprising a nucleic acid encoding an alpha-1-antitrypsin serpin domain operatively linked to a nucleotide sequence encoding a human serum albumin or a human serum albumin binding domain. In some embodiments, the human serum albumin binding domain comprises an antibody. In some embodiments, the antibody comprises a single domain antibody.

In some embodiments, the disclosure features a nucleic acid molecule that includes a fragment of an alpha-1-antitrypsin serpin domain encoding sequence and a fragment of a human serum albumin binding domain or a human serum albumin domain encoding sequence. In some embodiments, the nucleotide sequence encodes a fragment or variant of the alpha-1-antitrypsin of SEQ ID NO: 1 and/or a fragment of a human serum albumin binding domain or a human serum albumin domain encoding sequence of SEQ ID NO: 3 or SEQ ID NO: 5 or any of SEQ NOs: 22-36.

In some embodiments, the nucleic acid comprises a vector as will be set forth below.

### Domains, Domain Order, and Linkers

In some embodiments, the recombinant proteins of the invention have one human serum albumin domain or one human serum albumin binding domain and one alpha1-antitrypsin serum domain. In some embodiments, multiple domains of each protein, however, may be used to make a recombinant protein of the invention. Similarly, more than one alpha-1-antitrypsin serum domain may be used to make a recombinant protein of the invention. For instance, an alpha-1-antitrypsin serum domain may be fused to both the N- and C-terminal ends of the human serum albumin domain or the human serum albumin binding domain.

In some embodiments, the recombinant proteins do not contain a linker between the fused portions of the polypeptides (e.g., the portion of alpha-1-antitrypsin serpin domain is fused to the portion of the human serum albumin domain and/or human serum albumin binding domain). In some embodiments, the recombinant proteins include a linker peptide between the fused portions of the polypeptides. The linker can be a peptide linker. The linker can improve expression yield of the recombinant protein. The linker can improve and/or enhance biological activity of the alpha-1-antitrypsin serpin domain.

One skilled in the art will appreciate that there are a great number of linkers that can be used for the present invention (See, for example, Chen at al., Fusion Protein Linkers: Property, Design, and Functionality, Adv Drug Deliv Rev. 2013 October 15; 65(10): 1357-1369 (2012), which is incorporated by reference herein in its entirety.) In some embodiments, the linker is a peptide having a sequence comprising, consisting of or, or consisting essentially of the set forth in SEQ ID NO: 63.

### Compositions

The disclosure provides a pharmaceutical composition comprising any of the recombinant proteins set forth herein. Pharmaceutical compositions may take the form of any acceptable pharmaceutical formulation. Pharmaceutical compositions can be formulated in a variety of different forms, such as liquid, semi-solid, and solid dosage forms, such as liquid solutions (e.g., injectable and infusible solutions), dispersions or suspensions, tablets, pills, powders, liposomes, and suppositories. The preferred form can depend on the intended mode of administration and therapeutic application.

Exemplary pharmaceutical compositions are described below. The pharmaceutical compositions include those suitable for parenteral (including intravenous, subcutaneous, intradermal, intramuscular, and intraarticular), topical (including dermal, transdermal, transmucosal, buccal, sublingual, and intraocular), and rectal administration, although the most suitable route may depend upon, for example, the condition and disorder of the recipient.

The pharmaceutical compositions described herein can be administered systemically, e.g., parenteral (e.g., intravenous, subcutaneous, intradermal, intramuscular, and intraarticular). The pharmaceutical compositions described herein can be administered locally, e.g., administered to an area affected by the condition or disorder the pharmaceutical composition is being administered to treat.

Compositions for parenteral administration include aqueous and non-aqueous sterile injection solutions that may contain anti-oxidants, buffers, bacteriostats, and solutes that render the composition isotonic with the blood of the intended recipient and aqueous and non-aqueous sterile suspensions that may include suspending agents and thickening agents. The composition may be presented in unit-dose or multi-dose containers, for example sealed ampoules and vials, and may be stored in a freeze-dried (lyophilized) condition requiring only the addition of the sterile liquid carrier, for example saline or water-for-injection, immediately prior to use. Extemporaneous injection solutions and suspensions may be prepared from sterile powders, granules, and tablets. Exemplary compositions for parenteral administration include injectable solutions or suspensions which can contain, for example, suitable non-toxic, parenterally acceptable diluents, or solvents, such as EDTA, mannitol, 1,3-butanediol, water, Ringer's solution, an isotonic sodium chloride solution, or other suitable dispersing or wetting and suspending agents. The compositions may contain pharmaceutically acceptable substances or adjuvants, including, but not limited to, EDTA, e.g., 0.5 mM EDTA; pH adjusting and buffering agents and/or tonicity adjusting agents, e.g., sodium acetate, sodium lactate, sodium chloride, potassium chloride, calcium chloride, sorbitan monolaurate, minor amounts of non-toxic auxiliary substances, such as wetting, or emulsifying agents or preservatives.

### Treatment

In some embodiments, the disclosure features a method of treating a disorder associated with an alpha-1 antitrypsin deficiency, the method comprising administering to a patient or subject in need thereof having a disorder associated with an alpha-1-antitrypsin deficiency an effective amount of a recombinant protein or pharmaceutical compositions provided herein. In some embodiments, the disclosure features a method of prophylactically treating a disorder associated with an alpha-1 antitrypsin deficiency, the method comprising administering to a patient or subject in need thereof having a disorder associated with an alpha-1-antitrypsin deficiency an effective amount of a recombinant protein or pharmaceutical compositions as set forth herein. In some embodiments, the recombinant protein or pharmaceutical compositions may be administered to a patient in need thereof alone or in combination with another compound.

In some embodiments, administration of the recombinant protein or pharmaceutical compositions is administered about once a week or at longer intervals than about once a week, about once every 10 days or at longer intervals than about once every 10 days, about once every 15 days or at longer intervals than about once every 15 days, about once every 20 days or at longer intervals than about once every 20 days, about once every 25 days or at longer intervals than about once every 25 days, about once every month or at longer intervals than about once a month, about once every two months or at longer intervals than about once every two months, or about once every three months or at longer intervals than about once every three months.

In some embodiments, the subject has been diagnosed with or is at risk of being diagnosed with an alpha-1-antitrypsin deficiency. In some embodiments, the alpha-1-antitrypsin deficiency shows evidence of liver disease or evidence of lung disease. In some embodiments, the alpha-1-antitrypsin deficiency occurs in a person that has no apparent cause of liver disease or lung disease. In some embodiments, the evidence of liver disease or evidence of lung disease is at least one selected from the group consisting of emphysema, hepatic failure, hepatitis, hepatomology, jaundice, cirrhosis, nephratic syndrome, autosomal recessive inheritance, COPD-like symptoms, dyspnea, elevated hepatic transaminases, and hepatocellular carcinoma.

In some embodiments, the alpha-1-antitrypsin deficiency is caused by deficiency mutations in the SERPINA1 gene. More than 120 deficiency mutations in the SERPINA1 gene have been identified and may be useful for diagnosis according to the invention. Some deficiency mutations do not affect the production of alpha-1 antitrypsin while others cause a shortage or deficiency of the protein. In some embodiments, the deficiency mutations in the SERPINA1 gene replace the amino acid glutamic acid with the amino acid lysine at protein position 342 according to SEQ ID NO: 1.

Abnormal alpha-1 antitrypsin proteins may bind together to form a large molecule that cannot leave the liver. The accumulation of these polymers often results in liver damage. In addition, lung tissue is destroyed because not enough alpha-1 antitrypsin is available to protect against neutrophil elastase or PR3. Polymers of alpha-1 antitrypsin may also contribute to excessive inflammation, which may explain some of the other features of alpha-1 antitrypsin deficiency, such as a skin condition called panniculitis. Other SERPINA1 deficiency mutations lead to the production of an abnormally small form of alpha-1 antitrypsin that is quickly broken down in the liver. As a result, little or no alpha-1 antitrypsin is available in the lungs. While the liver remains healthy in individuals with these deficiency mutations, the lungs are left unprotected from neutrophil elastase or PR3.

In some embodiments, the deficiency mutation is a loss-of-function mutation. In some embodiments, the deficiency mutation results in a stop codon. In some embodiments, the deficiency mutation results in an amino acid substitution. In some embodiments, the deficiency mutation is a point mutation, insertion, deletion, and/or substitution. In some embodiments, the deficiency mutation is a point mutation. In some embodiments, the deficiency mutation is a deletion. The deletion can involve only one or a few base pairs, multiple exons, or the entire gene. In some embodiments, the deficiency mutation is a whole gene deletion. In some embodiments, the deficiency mutation is a partial gene deletion. In some embodiments, the deficiency mutation results in intronic changes that affect splicing. In some embodiments, the deficiency mutation is an alteration of the 3′ untranslated region of the gene. In some embodiments, the deficiency mutation is a gross chromosomal rearrangement. In some embodiments, the deficiency mutation results in truncation of the SERPINA1 gene product.

In some embodiments, the subject has aberrant expression of the alpha-1-antitrypsin protein compared to a reference standard. In some embodiments, the reference standard is the level of expression of the alpha-1-antitrypsin protein in a subject who does not have the disorder associated with alpha-1-antitrypsin deficiency. In some embodiments, the subject has decreased or substantially no expression of the alpha-1-antitrypsin protein compared to a reference standard. In some embodiments, the reference standard is the level of expression of the alpha-1-antitrypsin protein in a subject who does not have the disorder associated with alpha-1-antitrypsin deficiency.

In some embodiments, the alpha-1-antitrypsin serpin domain is a normal alpha-1-antitrypsin domain allele (See Crystal, R. G. The alpha-1-antitrypsin gene and its deficiency states. Trends Genet. 5: 411-417, 1989. [PubMed: 2696185], which is incorporated by reference in its entirety herein.) In some embodiments, the allele is an allele with a valine at position 213 (M1V, 107400.0002) and that with alanine at position 213 (MiA; 107400.0001) according to SEQ ID NO: 1 (See Nukiwa, T., Brantly, M. L., Ogushi, F., Fells, G. A., Crystal, R. G. Characterization of the gene and protein of the common alpha-1-antitrypsin normal M2 allele. Am. J. Hum. Genet. 43: 322-330, 1988. [PubMed: 2901226], which is incorporated by reference in its entirety herein.) In some embodiments, the allele is a deficiency allele or a null allele.

In some embodiments, the subject has been diagnosed with or is at risk of a disorder associated with an alpha-1-antitrypsin deficiency. In some embodiments, the subject has a mutation in the gene encoding an alpha-1-antitrypsin domain. In some embodiments, the mutation is a mutation in one of the exons of alpha-1-antitrypsin. In some embodiments, the mutation is a mutation in exon 1 of alpha-1-antitrypsin, the 5-prime coding region of exon 2 of alpha-1-antitrypsin, or the 3-prime portion of exon 5 of alpha-1-antitrypsin (See Long, G. L., Chandra, T., Woo, S. L. C., Davie, E. W., Kurachi, K. Complete sequence of the cDNA for human alpha-1-antitrypsin and the gene for the S variant. Biochemistry 23: 4828-4837, 1984. [PubMed: 6093867], which is incorporated by reference in its entirety herein.)

In some embodiments, the recombinant protein or pharmaceutical compositions is administered intravenously. In some embodiments, the recombinant protein or pharmaceutical compositions is administered intradermally. In some embodiments, the recombinant protein or pharmaceutical compositions is administered topically. In some embodiments, the recombinant protein or pharmaceutical compositions is administered subcutaneously. In some embodiments, the recombinant protein or pharmaceutical compositions is administered intrathecally. In some embodiments, the recombinant protein or pharmaceutical compositions is administered intrathecally.

### Vectors and Host Cells and Expression of Polypeptides

In some embodiments, the nucleic acid is a vector. In some embodiments, the vector comprises a host cell. Some embodiments are drawn to a method of making a recombinant protein comprising culturing the host cell with one of the nucleic acids provided herein and collecting the recombinant protein. In some embodiments, the recombinant protein is collected using an alpha-1-antitrypsin-Fc capture select media at neutral pH.

The recombinant proteins described herein may be produced, e.g., as recombinant molecules by secretion from any suitable host cell. Many expression systems are known and may be used, including bacteria (for example E. coli and Bacillus subtilis), yeasts (for example Saccharomyces cerevisiae, Kluyveromyces lactis and Pichia pastoris, filamentous fungi (for example Aspergillus), plant cells, animal cells, and insect cells.

In some embodiments, the host cell is a eukaryotic cell. In some embodiments, the host cell is a mammalian cell. In some embodiments, the host cell is selected from the group consisting of CHO cells, BUK cells, COS-7 cells, L cells, C127 cells, and 3T3 cells. In some embodiments, the host cell is a CHO cell.

In some embodiments, the host cell is a yeast cell. Exemplary genera of yeast which can be used as hosts described herein include but are not limited to Pichia, Saccharomyces, Kluyveromyces, Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Botryoascus, Sporidiobolus, Endomycopsis, and the like. Examples of Saccharomyces spp, are S. cerevisiae, S. italicus and S. rouxii. Yeast strains suitable for the production of fusion polypeptides described herein can include, but are not limited to D88, DXY1 and BXP10.

In addition to the transformed host cells themselves, also described herein is a culture of those cells, preferably a monoclonal (clonally homogeneous) culture, or a culture derived from a monoclonal culture, in a nutrient medium. If the recombinant protein is secreted, the medium will contain the recombinant protein, with the cells, or without the cells if they have been filtered or centrifuged away.

The recombinant proteins can be produced in conventional ways, for example from a coding sequence inserted in the host chromosome or on a free plasmid. The host cells are transformed with a coding sequence for the recombinant protein by any method known in the art, for example electroporation. Successfully transformed cells, i.e., cells that contain a DNA construct described herein, can be identified by techniques known to those of skill in the art. For example, cells resulting from the introduction of an expression construct can be grown to produce the recombinant protein. Cells can be harvested and lysed and their DNA content examined for the presence of the DNA using methods known in the art. The presence of the recombinant protein in the supernatant can be detected using any method known in the art (e.g. an antibody based detection method).

Also disclosed herein are plasmid vectors comprising the nucleic acids used to express the recombinant proteins described herein. In order to express the recombinant proteins described herein, the nucleotide sequence encoding the appropriate recombinant proteins, or a functional equivalent, can be inserted into a suitable vector. A suitable vector contains the necessary and appropriate transcriptional and translational control sequences for expression of the inserted nucleic acid sequence. Standard methods, known to those skilled in the art, may be used to construct the recombinant expression vectors containing the nucleic acid sequences described herein. These methods include, but are not limited to, in vitro recombinant techniques, synthetic techniques, and in vivo recombination/genetic recombination; the choice of method depends on the nature of the specific nucleotide fragments and may be determined by persons skilled in the art.

Suitable vectors for use herein may contain an origin of replication and a restriction endonuclease sequence site. Persons skilled in the art would have knowledge of suitable origin of replication and restriction endonuclease sequences for use in the host cell. Suitable vectors for use herein may contain sequence elements to aid transcription, including, but not limited to, promoter and enhancer elements. Persons skilled in the art would have knowledge of various transcriptional control elements, including but not limited to, promoters, inducible promoters, and enhancer elements, that would be suitable in the host cell.

Suitable vectors for use herein may also contain a selectable marker gene that encodes a product necessary for the host cell to grow and survive under specific conditions, aiding in the selection of host cells into which the vector has been introduced. Typical selection genes may include, but not be limited to, genes encoding a protein that confers resistance to an antibiotic, drug, or toxin (e.g. tetracycline, ampicillin, neomycin, hygromycin, etc). Persons skilled in the art would have knowledge of coding sequences for suitable selectable markers and reporter genes for use in the host cell.

Expression vectors described herein can be introduced into host cells via conventional transformation or transfection techniques. Transformation and transfection techniques include, but are not limited to, calcium phosphate or calcium chloride coprecipitation, DEAE-dextran-mediated transfection, lipofectamine, electroporation, microinjection, and viral mediated transfection (See, U.S. Pat. No. 6,632,637, which is incorporated by reference herein in its entirety.) Persons skilled in the art would have knowledge of suitable transformation and transfection methods based on the host cell/vector combination. For long term, high yield production of recombinant proteins, stable expression of the recombinant protein may be preferred. Host cells that stably express the recombinant protein may be engineered. Yeast plasmid vectors can include but are not limited to pRS403-406, pRS413-416, pRS403, pRS404, pRS405, pRS406, pRS413-416, pPPC0005, pScCHSA, pScNHSA, and pC4.

In another embodiment, the recombinant mammalian expression vector is capable of directing expression of the polypeptide preferentially in a particular cell type (e.g., tissue-specific regulatory elements are used I to express the polypeptide). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), T cell promoters (Winoto and Baltimore (1989) EMBO J. 8:729-733) and immunoglobulin promoters (Banerji et al. (1983) Cell 33:729-740 and Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the alpha-fetoprotein promoter (Campes and Tilghman (1989) Genes Dev. 3:537-546). Each of the above-identified references are incorporated by reference in their entirety, including any drawings.

Recombinant proteins described herein can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, hydrophobic charge interaction chromatography, and lectin chromatography. In some embodiments, high performance liquid chromatography (“HPLC”) is employed for purification. Polypeptides produced and recovered by recombinant and molecular biology methods described herein may be purified according to standard protocols known in the art (e.g., dialysis, ion exchange chromatography, affinity chromatography, SDS gel electrophoresis, etc). The polypeptides and/or fusion polypeptides described herein may be purified to homogeneity by ion exchange chromatography, hydrophobic interaction chromatography, reverse phase chromatography, or gel filtration. Depending upon the host employed in a recombinant production procedure, the polypeptides of the present invention may be glycosylated or may be non-glycosylated. In some embodiments, the recombinant protein will be purified with an alpha-1-antitrypsin-Fc capture select media at neutral pH.

In some embodiments, the nucleic acids encoding the recombinant protein described herein is optimized for expression in yeast. Exemplary genera of yeast which can be used as hosts for expressing the fusion polypeptides described herein include but are not limited to Pichia, Saccharomyces, Kluyveromyces, Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Botryoascus, Sporidiobolus, Endomycopsis, and the like. Yeast strains suitable for the production of fusion polypeptides described herein can include, but are not limited to D88, DXY1 and BXP10. Yeast plasmid vectors can include but are not limited to pRS403-406, pRS413-416, pRS403, pRS404, pRS405, pRS406, pRS413-416, pPPC0005, pScCHSA, pScNHSA, and pC4. Vectors for making albumin fusion proteins for expression in yeast include pPPC0005, pScCHSA, pScNHSA, and pC4:HSA.

In some embodiments, the nucleic acids encoding the recombinant protein described herein is expressed in Saccharomyces. Preferred exemplary species of Saccharomyces include S. cerevisiae, S. italicus, S. diastaticus, and Zygosaccharomyces rouxii. Preferred exemplary species of Kluyveromyces include K fragilis and K lactis. Preferred exemplary species of Hansenula include H. polymorpha (now Pichia angusta), H. anomala (now Pichia anomala) and Pichia capsulata.

Other organisms may be used for expression. Additional exemplary species of Pichia include P. pastoris. Exemplary species of Aspergillus include A. niger and A. nidulans. Preferred exemplary species of Yarrowia include Y. lipolytica. Many preferred yeast species are available from the ATCC® (American Type Culture Collection).

TABLE S
Sequences.

| SEQ | | |
|---|---|---|
| ID | | |
| NO: | Region | Sequence |
| 1 | Alpha-1- | EDPQGDAAQKTDTSHHDQDHPT |
| | antitrypsin | FNKITPNLAEFAFSLYRQLAHQ |
| | | SNSTNIFFSPVSIATAFAMLSL |
| | | GTKADTHDEILEGLNFNLTEIP |
| | | EAQIHEGFQELLRTLNQPDSQL |
| | | QLTTGNGLFLSEGLKLVDKFLE |
| | | DVKKLYHSEAFTVNFGDTEEAK |
| | | KQINDYVEKGTQGKIVDLVKEL |
| | | DRDTVFALVNYIFFKGKWERPF |
| | | EVKDTEEEDFHVDQVTTVKVPM |
| | | MKRLGMFNIQHCKKLSSWVLLM |
| | | KYLGNATAIFFLPDEGKLQHLE |
| | | NELTHDIITKFLENEDRRSASL |
| | | HLPKLSITGTYDLKSVLGQLGI |
| | | TKVFSNGADLSGVTEEAPLKLS |
| | | KAVHKAVLTIDEKGTEAAGAMF |
| | | LEAIPMSIPPEVKFNKPFVFLM |
| | | IEQNTKSPLFMGKWNPTQK |
| 2 | Alpha-1- | MPSSVSWGILLLAGLCCLVPVS |
| | antitrypsin | LA |
| 3 | Human | MKWVTFISLLFLFSSAYSRGVF |
| | Albumin | RRDAHKSEVAHRFKDLGEENFK |
| | | ALVLIAFAQYLQQCPFEDHVKL |
| | | VNEVTEFAKTCVADESAENCDK |
| | | SLHTLFGDKLCTVATLRETYGE |
| | | MADCCAKQEPERNECFLQHKDD |
| | | NPNLPRLVRPEVDVMCTAFHDN |
| | | EETFLKKYLYEIARRHPYFYAP |
| | | ELLFFAKRYKAAFTECCQAADK |
| | | AACLLPKLDELRDEGKASSAKQ |
| | | GLKCASLQKFGERAFKAWAVAR |
| | | LSQRFPKAEFAEVSKLVTDLTK |
| | | VHTECCHGDLLECADDRADLAK |
| | | YICENQDSISSKLKECCEKPLL |
| | | EKSHCIAEVENDEMPADLPSLA |
| | | ADFVGSKDVCKNYAEAKDVFLG |
| | | MFLYEYARRHPDYSVVLLLRLA |
| | | KTYETTLEKCCAAADPHECYAK |
| | | VFDEFKPLVEEPQNLIKQNCEL |
| | | FEQLGEYKFQNALLVRYTKKVP |
| | | QVSTPTLVEVSRNLGKVGSKCC |
| | | KHPEAKRMPCAEDCLSVFLNQL |
| | | CVLHEKTPVSDRVT |
| 4 | Human | MKWVTFISLLFLFSSAYS |
| | Albumin | |
| | Leader | |
| 5 | Human Serum | RGVFRRDAHKSEVAHRFKDLGE |
| | Albumin | ENFKALVLIAFAQYLQQCPFED |
| | | HVKLVNEVTEFAKTCVADESAE |
| | | NCDKSLHTLFGDKLCTVATLRE |
| | | TYGEMADCCAKQEPERNECFLQ |
| | | HKDDNPNLPRLVRPEVDVMCTA |
| | | FHDNEETFLKKYLYEIARRHPY |
| | | FYAPELLFFAKRYKAAFTECCQ |
| | | AADKAACLLPKLDELRDEGKAS |
| | | SAKQGLKCASLQKFGERAFKAW |
| | | AVARLSQRFPKAEFAEVSKLVT |
| | | DLTKVHTECCHGDLLECADDRA |
| | | DLAKYICENQDSISSKLKECCE |
| | | KPLLEKSHCIAEVENDEMPADL |
| | | PSLAADFVGSKDVCKNYAEAKD |
| | | VFLGMFLYEYARRHPDYSVVLL |
| | | LRLAKTYETTLEKCCAAADPHE |
| | | CYAKVFDEFKPLVEEPQNLIKQ |
| | | NCELFEQLGEYKFQNALLVRYT |
| | | KKVPQVSTPTLVEVSRNLGKVG |
| | | SKCCKHPEAKRMPCAEDCLSVF |
| | | LNQLCVLHEKTPVSDRVT |
| 6 | CDR1 | LNLMG |
| 7 | CDR1 | INLLG |
| 8 | CDR1 | SFGMS |
| 9 | CDR1 | NYWMY |
| 10 | CDR2 | TCITVGDSTNYADSVKG |
| 11 | CDR2 | TITVGDSTSYADSVKG |
| 12 | CDR2 | SISGSGSDTLYADSVKG |
| 13 | CDR2 | SINGRGDDTRYADSVKG |
| 14 | CDR2 | AISADSSTKNYADSVKG |
| 15 | CDR2 | AISADSSDKRYADSVKG |
| 16 | CDR2 | RDISTGGGYSYYADSVKG |
| 17 | CDR3 | RRTWHSEL |
| 18 | CDR3 | GGSLSR |
| 19 | CDR3 | GRSVSRS |
| 20 | CDR3 | GRGSP |
| 21 | CDR3 | DREAQVDTLDFDY |
| 22 | SDA | AVQLVESGGGLVQGGGSLRLAC |
| | | AASERIFDLNLMGWYRQGPGNE |
| | | RELVATCITVGDSTNYADSVKG |
| | | RFTISMDYTKQTVYLHMNSLRP |
| | | EDTGLYYCKIRRTWHSELWGQG |
| | | TQVTVSS |
| 23 | SDA | EVQLVESGGGLVQEGGSLRLAC |
| | | AASERIWDINLLGWYRQGPGNE |
| | | RELVATITVGDSTSYADSVKGR |
| | | FTISRDYDKNTLYLQMNSLRPE |
| | | DTGLYYCKIRRTWHSELWGQGT |
| | | QVTVSS |
| 24 | SDA | AVQLVESGGGLVQPGNSLRLSC |
| | | AASGFTFRSFGMSWVRQAPGKE |
| | | PEWVSSISGSGSDTLYADSVKG |
| | | RFTISRDNAKTTLYLQMNSLKP |
| | | EDTAVYYCTIGGSLSRSSQGTQ |
| | | VTVSS |
| 25 | SDA | AVQLVDSGGGLVQPGGSLRLSC |
| | | AASGFSFGSFGMSWVRQYPGKE |
| | | PEWVSSINGRGDDTRYADSVKG |
| | | RFSISRDNAKNTLYLQMNSLKP |
| | | EDTAEYYCTIGRSVSRSRTQGT |
| | | QVTVSS |
| 26 | SDA | AVQLVESGGGLVQPGGSLRLTC |
| | | TASGFTFRSFGMSWVRQAPGKD |
| | | QEWVSAISADSSTKNYADSVKG |
| | | RFTISRDNAKKMLYLEMNSLKP |
| | | EDTAVYYCVIGRGSPSSPGTQV |
| | | TVSS |
| 27 | SDA | QVQLAESGGGLVQPGGSLRLTC |
| | | TASGFTFGSFGMSWVRQAPGEG |
| | | LEWVSAISADSSDKRYADSVKG |
| | | RFTISRDNAKKMLYLEMNSLKS |
| | | EDTAVYYCVIGRGSPASQGTQV |
| | | TVSS |
| 28 | SDA | QVQLVESGGGLVQPGGSLRLSC |
| | | AASGFTFSNYWMYWVRVAPGKG |
| | | LERISRDISTGGGYSYYADSVK |
| | | GRFTISRDNAKNTLYLQMNSLK |
| | | PEDTALYYCAKDREAQVDTLDF |
| | | DYRGQGTQVTVSS |
| 29 | hSDA | EVQLVESGGGLVQPGGSLRLSC |
| | | AASGFTFRSFGMSWVRQAPGKE |
| | | PEWVSSISGSGSDTLYADSVKG |
| | | RFTISRDNAKTTLYLQMNSLKP |
| | | EDTAVYYCTIGGSLSRSSQGTQ |
| | | VTVSS |
| 30 | hSDA | EVQLVESGGGLVQPGGSLRLSC |
| | | AASGFTFSSFGMSWVRQAPGKE |
| | | PEWVSSISGSGSDTLYADSVKG |
| | | RFTISRDNAKTTLYLQMNSLKP |
| | | EDTAVYYCTIGGSLSRSSQGTQ |
| | | VTVSS |
| 31 | hSDA | EVQLVESGGGLVQPGGSLRLSC |
| | | AASGFTFRSFGMSWVRQAPGKG |
| | | LEWVSSISGSGSDTLYADSVKG |
| | | RFTISRDNAKTTLYLQMNSLKP |
| | | EDTAVYYCTIGGSLSRSSQGTQ |
| | | VTVSS |
| 32 | hSDA | EVQLVESGGGLVQPGNSLRLSC |
| | | AASGFTFRSFGMSWVRQAPGKG |
| | | LEWVSSISGSGSDTLYADSVKG |
| | | RFTISRDNAKTTLYLQMNSLKP |
| | | EDTAVYYCTIGGSLSRSSQGTL |
| | | VTVSS |
| 33 | hSDA | EVQLVESGGGLVQPGNSLRLSC |
| | | AASGFTFRSFGMSWVRQAPGKG |
| | | LEWVSSISGSGSDTLYADSVKG |
| | | RFTISRDNAKTTLYLQMNSLRP |
| | | EDTAVYYCTIGGSLSRSSQGTL |
| | | VTVSS |
| 34 | hSDA | EVQLVESGGGLVQPGNSLRLSC |
| | | AASGFTFSSFGMSWVRQAPGKG |
| | | LEWVSSISGSGSDTLYADSVKG |
| | | RFTISRDNAKTTLYLQMNSLRP |
| | | EDTAVYYCTIGGSLSRSSQGTL |
| | | VTVSS |
| 35 | hSDA | EVQLVESGGGLVQPGNSLRLSC |
| | | AASGFTFSSFGMSWVRQAPGKG |
| | | LEWVSSISGSGSDTLYADSVKG |
| | | RFTISRDNAKNTLYLQMNSLRP |
| | | EDTAVYYCTIGGSLSRSSQGTL |
| | | VTVSS |
| 36 | hSDA | EVQLVESGGGLVQPGNSLRLSC |
| | | AASGFTFSSFGMSWVRQAPGKG |
| | | LEWVSSISGSGSDTLYADSVKG |
| | | RFTISRDNAKNTLYLQMNSLRP |
| | | EDTAVYYCTIGGSLSRSGQGTL |
| | | VTVSS |
| 37 | FR1 | AVQLVESGGGLVQGGGSLRLAC |
| | | AASERIFD |
| 38 | FR1 | EVQLVESGGGLVQEGGSLRLAC |
| | | AASERIWD |
| 39 | FR1 | AVQLVESGGGLVQPGNSLRLSC |
| | | AASGFTFR |
| 40 | FR1 | AVQLVDSGGGLVQPGGSLRLSC |
| | | AASGFSFG |
| 41 | FR1 | AVQLVESGGGLVQPGGSLRLTC |
| | | TASGFTFR |
| 42 | FR1 | QVQLAESGGGLVQPGGSLRLTC |
| | | TASGFTFG |
| 43 | FR1 | QVQLVESGGGLVQPGGSLRLSC |
| | | AASGFTFS |
| 44 | FR2 | WYRQGPGNERELVA |
| 45 | FR2 | WVRQAPGKEPEWVS |
| 46 | FR2 | WVRQYPGKEPEWVS |
| 47 | FR2 | WVRQAPGKDQEWVS |
| 48 | FR2 | WVRQAPGEGLEWVS |
| 49 | FR2 | WVRVAPGKGLERIS |
| 50 | FR3 | RFTISMDYTKQTVYLHMNSLRP |
| | | EDTGLYYCKI |
| 51 | FR3 | RFTISRDYDKNTLYLQMNSLRP |
| | | EDTGLYYCKI |
| 52 | FR3 | RFTISRDNAKTTLYLQMNSLKP |
| | | EDTAVYYCTI |
| 53 | FR3 | RFSISRDNAKNTLYLQMNSLKP |
| | | EDTAEYYCTI |
| 54 | FR3 | RFTISRDNAKKMLYLEMNSLKP |
| | | EDTAVYYCVI |
| 55 | FR3 | RFTISRDNAKKMLYLEMNSLKS |
| | | EDTAVYYCVI |
| 56 | FR3 | RFTISRDNAKNTLYLQMNSLKP |
| | | EDTALYYCAK |
| 57 | FR4 | WGQGTQVTVSS |
| 58 | FR4 | SSQGTQVTVSS |
| 59 | FR4 | RTQGTQVTVSS |
| 60 | FR4 | SSPGTQVTVSS |
| 61 | FR4 | ASQGTQVTVSS |
| 62 | FR4 | RGQGTQVTVSS |
| 63 | Linker | GGSGGSGGSGGSGG |
| 64 | AAT- | MPSSVSWGILLLAGLCCLVPVS |
| | linkerAlbumin | LAEDPQGDAAQKTDTSHHDQD |
| | Fusion | HPTFNKITPNLAEFAFSLYRQL |
| | | AHQSNSTNIFFSPVSIATAFAM |
| | | LSLGTKADTHDEILEGLNFNLT |
| | | EIPEAQIHEGFQELLRTLNQPD |
| | | SQLQLTTGNGLFLSEGLKLVDK |
| | | FLEDVKKLYHSEAFTVNFGDTE |
| | | EAKKQINDYVEKGTQGKIVDLV |
| | | KELDRDTVFALVNYIFFKGKWE |
| | | RPFEVKDTEEEDFHVDQVTTVK |
| | | VPMMKRLGMFNIQHCKKLSSWV |
| | | LLMKYLGNATAIFFLPDEGKLQ |
| | | HLENELTHDIITKFLENEDRRS |
| | | ASLHLPKLSITGTYDLKSVLGQ |
| | | LGITKVFSNGADLSGVTEEAPL |
| | | KLSKAVHKAVLTIDEKGTEAAG |
| | | AMFLEAIPMSIPPEVKFNKPFV |
| | | FLMIEQNTKSPLFMGKVVNPTQ |
| | | KGGSGGSGGSGGSGGDAHKSEV |
| | | AHRFKDLGEENFKALVLIAFAQ |
| | | YLQQCPFEDHVKLVNEVTEFAK |
| | | TCVADESAENCDKSLHTLFGDK |
| | | LCTVATLRETYGEMADCCAKQE |
| | | PERNECFLQHKDDNPNLPRLVR |
| | | PEVDVMCTAFHDNEETFLKKYL |
| | | YEIARRHPYFYAPELLFFAKRY |
| | | KAAFTECCQAADKAACLLPKLD |
| | | ELRDEGKASSAKQRLKCASLQK |
| | | FGERAFKAWAVARLSQRFPKAE |
| | | FAEVSKLVTDLTKVHTECCHGD |
| | | LLECADDRADLAKYICENQDSI |
| | | SSKLKECCEKPLLEKSHCIAEV |
| | | ENDEMPADLPSLAADFVESKDV |
| | | CKNYAEAKDVFLGMFLYEYARR |
| | | HPDYSVVLLLRLAKTYETTLEK |
| | | CCAAADPHECYAKVFDEFKPLV |
| | | EEPQNLIKQNCELFEQLGEYKF |
| | | QNALLVRYTKKVPQVSTPTLVE |
| | | VSRNLGKVGSKCCKHPEAKRMP |
| | | CAEDYLSVVLNQLCVLHEKTPV |
| | | SDRVTKCCTESLVNRRPCFSAL |
| | | EVDETYVPKEFNAETFTFHADI |
| | | CTLSEKERQIKKQTALVELVKH |
| | | KPKATKEQLKAVMDDFAAFVEK |
| | | CCKADDKETCFAEEGKKLVAAS |
| | | QAALGL |
| 65 | Mutant | MPSSVSWGILLLAGLCCLVPVS |
| | AAT- | LA_EDPQGDAAQKTDTSHHDQD |
| | linkerAlbumin | HPTFNKITPNLAEFAFSLYRQL |
| | Fusion | AHQSNSTNIFFSPVSIATAFAM |
| | (C232S) | LSLGTKADTHDEILEGLNFNLT |
| | | EIPEAQIHEGFQELLRTLNQPD |
| | | SQLQLTTGNGLFLSEGLKLVDK |
| | | FLEDVKKLYHSEAFTVNFGDTE |
| | | EAKKQINDYVEKGTQGKIVDLV |
| | | KELDRDTVFALVNYIFFKGKWE |
| | | RPFEVKDTEEEDFHVDQVTTVK |
| | | VPMMKRLGMFNIQHSKKLSSWV |
| | | LLMKYLGNATAIFFLPDEGKLQ |
| | | HLENELTHDIITKFLENEDRRS |
| | | ASLHLPKLSITGTYDLKSVLGQ |
| | | LGITKVFSNGADLSGVTEEAPL |
| | | KLSKAVHKAVLTIDEKGTEAAG |
| | | AMFLEAIPMSIPPEVKFNKPFV |
| | | FLMIEQNTKSPLFMGKVVNPTQ |
| | | KGGSGGSGGSGGSGGDAHKSEV |
| | | AHRFKDLGEENFKALVLIAFAQ |
| | | YLQQCPFEDHVKLVNEVTEFAK |
| | | TCVADESAENCDKSLHTLFGDK |
| | | LCTVATLRETYGEMADCCAKQE |
| | | PERNECFLQHKDDNPNLPRLVR |
| | | PEVDVMCTAFHDNEETFLKKYL |
| | | YEIARRHPYFYAPELLFFAKRY |
| | | KAAFTECCQAADKAACLLPKLD |
| | | ELRDEGKASSAKQRLKCASLQK |
| | | FGERAFKAWAVARLSQRFPKAE |
| | | FAEVSKLVTDLTKVHTECCHGD |
| | | LLECADDRADLAKYICENQDSI |
| | | SSKLKECCEKPLLEKSHCIAEV |
| | | ENDEMPADLPSLAADFVESKDV |
| | | CKNYAEAKDVFLGMFLYEYARR |
| | | HPDYSVVLLLRLAKTYETTLEK |
| | | CCAAADPHECYAKVFDEFKPLV |
| | | EEPQNLIKQNCELFEQLGEYKF |
| | | QNALLVRYTKKVPQVSTPTLVE |
| | | VSRNLGKVGSKCCKHPEAKRMP |
| | | CAEDYLSVVLNQLCVLHEKTPV |
| | | SDRVTKCCTESLVNRRPCFSAL |
| | | EVDETYVPKEFNAETFTFHADI |
| | | CTLSEKERQIKKQTALVELVKH |
| | | KPKATKEQLKAVMDDFAAFVEK |
| | | CCKADDKETCFAEEGKKLVAAS |
| | | QAALGL |
| 66 | Mutant | MPSSVSWGILLLAGLCCLVPVS |
| | AAT- | LA_EDPQGDAAQKTDTSHHDQD |
| | linkerAlbumin | HPTFNKITPNLAEFAFSLYRQL |
| | Fusion | AHQSNSTNIFFSPVSIATAFAM |
| | (M351V, M358V) | LSLGTKADTHDEILEGLNFNLT |
| | | EIPEAQIHEGFQELLRTLNQPD |
| | | SQLQLTTGNGLFLSEGLKLVDK |
| | | FLEDVKKLYHSEAFTVNFGDTE |
| | | EAKKQINDYVEKGTQGKIVDLV |
| | | KELDRDTVFALVNYIFFKGKWE |
| | | RPFEVKDTEEEDFHVDQVTTVK |
| | | VPMMKRLGMFNIQHCKKLSSWV |
| | | LLMKYLGNATAIFFLPDEGKLQ |
| | | HLENELTHDIITKFLENEDRRS |
| | | ASLHLPKLSITGTYDLKSVLGQ |
| | | LGITKVFSNGADLSGVTEEAPL |
| | | KLSKAVHKAVLTIDEKGTEAAG |
| | | AVFLEAIPVSIPPEVKFNKPFV |
| | | FLMIEQNTKSPLFMGKVVNPTQ |
| | | KGGSGGSGGSGGSGGDAHKSEV |
| | | AHRFKDLGEENFKALVLIAFAQ |
| | | YLQQCPFEDHVKLVNEVTEFAK |
| | | TCVADESAENCDKSLHTLFGDK |
| | | LCTVATLRETYGEMADCCAKQE |
| | | PERNECFLQHKDDNPNLPRLVR |
| | | PEVDVMCTAFHDNEETFLKKYL |
| | | YEIARRHPYFYAPELLFFAKRY |
| | | KAAFTECCQAADKAACLLPKLD |
| | | ELRDEGKASSAKQRLKCASLQK |
| | | FGERAFKAWAVARLSQRFPKAE |
| | | FAEVSKLVTDLTKVHTECCHGD |
| | | LLECADDRADLAKYICENQDSI |
| | | SSKLKECCEKPLLEKSHCIAEV |
| | | ENDEMPADLPSLAADFVESKDV |
| | | CKNYAEAKDVFLGMFLYEYARR |
| | | HPDYSVVLLLRLAKTYETTLEK |
| | | CCAAADPHECYAKVFDEFKPLV |
| | | EEPQNLIKQNCELFEQLGEYKF |
| | | QNALLVRYTKKVPQVSTPTLVE |
| | | VSRNLGKVGSKCCKHPEAKRMP |
| | | CAEDYLSVVLNQLCVLHEKTPV |
| | | SDRVTKCCTESLVNRRPCFSAL |
| | | EVDETYVPKEFNAETFTFHADI |
| | | CTLSEKERQIKKQTALVELVKH |
| | | KPKATKEQLKAVMDDFAAFVEK |
| | | CCKADDKETCFAEEGKKLVAAS |
| | | QAALGL |
| 67 | Mutant | MPSSVSWGILLLAGLCCLVPVS |
| | AAT- | LA_EDPQGDAAQKTDTSHHDQD |
| | linkerAlbumin | HPTFNKITPNLAEFAFSLYRQL |
| | Fusion | AHQSNSTNIFFSPVSIATAFAM |
| | (M351V, M358L) | LSLGTKADTHDEILEGLNFNLT |
| | | EIPEAQIHEGFQELLRTLNQPD |
| | | SQLQLTTGNGLFLSEGLKLVDK |
| | | FLEDVKKLYHSEAFTVNFGDTE |
| | | EAKKQINDYVEKGTQGKIVDLV |
| | | KELDRDTVFALVNYIFFKGKWE |
| | | RPFEVKDTEEEDFHVDQVTTVK |
| | | VPMMKRLGMFNIQHCKKLSSWV |
| | | LLMKYLGNATAIFFLPDEGKLQ |
| | | HLENELTHDIITKFLENEDRRS |
| | | ASLHLPKLSITGTYDLKSVLGQ |
| | | LGITKVFSNGADLSGVTEEAPL |
| | | KLSKAVHKAVLTIDEKGTEAAG |
| | | AVFLEAIPLSIPPEVKFNKPFV |
| | | FLMIEQNTKSPLFMGKVVNPTQ |
| | | KGGSGGSGGSGGSGGDAHKSEV |
| | | AHRFKDLGEENFKALVLIAFAQ |
| | | YLQQCPFEDHVKLVNEVTEFAK |
| | | TCVADESAENCDKSLHTLFGDK |
| | | LCTVATLRETYGEMADCCAKQE |
| | | PERNECFLQHKDDNPNLPRLVR |
| | | PEVDVMCTAFHDNEETFLKKYL |
| | | YEIARRHPYFYAPELLFFAKRY |
| | | KAAFTECCQAADKAACLLPKLD |
| | | ELRDEGKASSAKQRLKCASLQK |
| | | FGERAFKAWAVARLSQRFPKAE |
| | | FAEVSKLVTDLTKVHTECCHGD |
| | | LLECADDRADLAKYICENQDSI |
| | | SSKLKECCEKPLLEKSHCIAEV |
| | | ENDEMPADLPSLAADFVESKDV |
| | | CKNYAEAKDVFLGMFLYEYARR |
| | | HPDYSVVLLLRLAKTYETTLEK |
| | | CCAAADPHECYAKVFDEFKPLV |
| | | EEPQNLIKQNCELFEQLGEYKF |
| | | QNALLVRYTKKVPQVSTPTLVE |
| | | VSRNLGKVGSKCCKHPEAKRMP |
| | | CAEDYLSVVLNQLCVLHEKTPV |
| | | SDRVTKCCTESLVNRRPCFSAL |
| | | EVDETYVPKEFNAETFTFHADI |
| | | CTLSEKERQIKKQTALVELVKH |
| | | KPKATKEQLKAVMDDFAAFVEK |
| | | CCKADDKETCFAEEGKKLVAAS |
| | | QAALGL |
| 68 | Mutant | MPSSVSWGILLLAGLCCLVPVS |
| | AAT- | LA_EDPQGDAAQKTDTSHHDQD |
| | linkerAlbumin | HPTFNKITPNLAEFAFSLYRQL |
| | Fusion | AHQSNSTNIFFSPVSIATAFAM |
| | (C232S, M351V, | LSLGTKADTHDEILEGLNFNLT |
| | M358L) | EIPEAQIHEGFQELLRTLNQPD |
| | | SQLQLTTGNGLFLSEGLKLVDK |
| | | FLEDVKKLYHSEAFTVNFGDTE |
| | | EAKKQINDYVEKGTQGKIVDLV |
| | | KELDRDTVFALVNYIFFKGKWE |
| | | RPFEVKDTEEEDFHVDQVTTVK |
| | | VPMMKRLGMFNIQHSKKLSSWV |
| | | LLMKYLGNATAIFFLPDEGKLQ |
| | | HLENELTHDIITKFLENEDRRS |
| | | ASLHLPKLSITGTYDLKSVLGQ |
| | | LGITKVFSNGADLSGVTEEAPL |
| | | KLSKAVHKAVLTIDEKGTEAAG |
| | | AVFLEAIPLSIPPEVKFNKPFV |
| | | FLMIEQNTKSPLFMGKVVNPTQ |
| | | KGGSGGSGGSGGSGGDAHKSEV |
| | | AHRFKDLGEENFKALVLIAFAQ |
| | | YLQQCPFEDHVKLVNEVTEFAK |
| | | TCVADESAENCDKSLHTLFGDK |
| | | LCTVATLRETYGEMADCCAKQE |
| | | PERNECFLQHKDDNPNLPRLVR |
| | | PEVDVMCTAFHDNEETFLKKYL |
| | | YEIARRHPYFYAPELLFFAKRY |
| | | KAAFTECCQAADKAACLLPKLD |
| | | ELRDEGKASSAKQRLKCASLQK |
| | | FGERAFKAWAVARLSQRFPKAE |
| | | FAEVSKLVTDLTKVHTECCHGD |
| | | LLECADDRADLAKYICENQDSI |
| | | SSKLKECCEKPLLEKSHCIAEV |
| | | ENDEMPADLPSLAADFVESKDV |
| | | CKNYAEAKDVFLGMFLYEYARR |
| | | HPDYSVVLLLRLAKTYETTLEK |
| | | CCAAADPHECYAKVFDEFKPLV |
| | | EEPQNLIKQNCELFEQLGEYKF |
| | | QNALLVRYTKKVPQVSTPTLVE |
| | | VSRNLGKVGSKCCKHPEAKRMP |
| | | CAEDYLSVVLNQLCVLHEKTPV |
| | | SDRVTKCCTESLVNRRPCFSAL |
| | | EVDETYVPKEFNAETFTFHADI |
| | | CTLSEKERQIKKQTALVELVKH |
| | | KPKATKEQLKAVMDDFAAFVEK |
| | | CCKADDKETCFAEEGKKLVAAS |
| | | QAALGL |
| 69 | Wild Type | tgggcaggaactgggcactgtg |
| | AAT | cccagggcatgcactgcctcca |
| | Nucleic Acid | cgcagcaaccctcagagtcctg |
| | | agctgaaccaagaaggaggagg |
| | | gggtcgggcctccgaggaaggc |
| | | ctagccgctgctgctgccagga |
| | | attccaggttggaggggcggca |
| | | acctcctgccagccttcaggcc |
| | | actctcctgtgcctgccagaag |
| | | agacagagcttgaggagagctt |
| | | gaggagagcaggaaaggacaat |
| | | gccgtcttctgtctcgtggggc |
| | | atcctcctgctggcaggcctgt |
| | | gctgcctggtccctgtctccct |
| | | ggctgaggatccccagggagat |
| | | gctgcccagaagacagatacat |
| | | cccaccatgatcaggatcaccc |
| | | aaccttcaacaagatcaccccc |
| | | aacctggctgagttcgccttca |
| | | gcctataccgccagctggcaca |
| | | ccagtccaacagcaccaatatc |
| | | ttcttctccccagtgagcatcg |
| | | ctacagcctttgcaatgctctc |
| | | cctggggaccaaggctgacact |
| | | cacgatgaaatcctggagggcc |
| | | tgaatttcaacctcacggagat |
| | | tccggaggctcagatccatgaa |
| | | ggcttccaggaactcctccgta |
| | | ccctcaaccagccagacagcca |
| | | gctccagctgaccaccggcaat |
| | | ggcctgttcctcagcgagggcc |
| | | tgaagctagtggataagttttt |
| | | ggaggatgttaaaaagttgtac |
| | | cactcagaagccttcactgtca |
| | | acttcggggacaccgaagaggc |
| | | caagaaacagatcaacgattac |
| | | gtggagaagggtactcaaggga |
| | | aaattgtggatttggtcaagga |
| | | gcttgacagagacacagttttt |
| | | gctctggtgaattacatcttct |
| | | ttaaaggcaaatgggagagacc |
| | | ctttgaagtcaaggacaccgag |
| | | gaagaggacttccacgtggacc |
| | | aggtgaccaccgtgaaggtgcc |
| | | tatgatgaagcgtttaggcatg |
| | | tttaacatccagcactgtaaga |
| | | agctgtccagctgggtgctgct |
| | | gatgaaatacctgggcaatgcc |
| | | accgccatcttcttcctgcctg |
| | | atgaggggaaactacagcacct |
| | | ggaaaatgaactcacccacgat |
| | | atcatcaccaagttcctggaaa |
| | | atgaagacagaaggtctgccag |
| | | cttacatttacccaaactgtcc |
| | | attactggaacctatgatctga |
| | | agagcgtcctgggtcaactggg |
| | | catcactaaggtcttcagcaat |
| | | ggggctgacctctccggggtca |
| | | cagaggaggcacccctgaagct |
| | | ctccaaggccgtgcataaggct |
| | | gtgctgaccatcgacgagaaag |
| | | ggactgaagctgctggggccat |
| | | gtttttagaggccatacccatg |
| | | tctatcccccccgaggtcaagt |
| | | tcaacaaaccctttgtcttctt |
| | | aatgattgaacaaaataccaag |
| | | tctcccctcttcatgggaaaag |
| | | tggtgaatcccacccaaaaata |
| | | actgcctctcgctcctcaaccc |
| | | ctcccctccatccctggccccc |
| | | tccctggatgaattaaagaagg |
| | | gttgagctggtccctgcctgca |
| | | tgtgactgtaaatccctcccat |
| | | gttttctctgagtctccctttg |
| | | cctgctgaggctgtatgtgggc |
| | | tccaggtaacagtgctgtcttc |
| | | gggccccctgaactgtgttcat |
| | | ggagcatctggctgggtaggca |
| | | catgctgggcttgaatccaggg |
| | | gggactgaatcctcagcttacg |
| | | gacctgggcccatctgtttctg |
| | | gagggctccagtcttccttgtc |
| | | ctgtcttggagtccccaagaag |
| | | gaatcacaggggaggaaccaga |
| | | taccagccatgaccccaggctc |
| | | caccaagcatcttcatgtcccc |
| | | ctgctcatcccccactcccccc |
| | | cacccagagttgctcatcctgc |
| | | cagggctggctgtgcccacccc |
| | | aaggctgccctcctgggggccc |
| | | cagaactgcctgatcgtgccgt |
| | | ggcccagttttgtggcatctgc |
| | | agcaacacaagagagaggacaa |
| | | tgtcctcctcttgactcgggcc |
| | | ctgcacctctcaggcacttctg |
| | | gaaaatgactgaggcagattct |
| | | tcctgaagcccattctccatgg |
| | | ggcaacaaggacacctattctg |
| | | tccttgtccttccatcgctgcc |
| | | ccagaaagcctcacatatctcc |
| | | gtttagaatcaggtcccttctc |
| | | cccagatgaagaggagggtctc |
| | | tgctttgttttctctatctcct |
| | | cctcagacttgaccaggcccag |
| | | caggccccagaagaccattacc |
| | | ctatatcccttctcctccctag |
| | | tcacatggccataggcctgctg |
| | | atggctcaggaaggccattgca |
| | | aggactcctcagctatgggaga |
| | | ggaagcacatcacccattgacc |
| | | cccgcaacccctccctttcctc |
| | | ctctgagtcccgactggggcca |
| | | catgcagcctgacttctttgtg |
| | | cctgttgctgtccctgcagtct |
| | | tcagagggccaccgcagctcca |
| | | gtgccacggcaggaggctgttc |
| | | ctgaatagcccctgtggtaagg |
| | | gccaggagagtccttccatcct |
| | | ccaaggccctgctaaaggacac |
| | | agcagccaggaagtcccctggg |
| | | cccctagctgaaggacagcctg |
| | | ctccctccgtctctaccaggaa |
| | | tggccttgtcctatggaaggca |
| | | ctgccccatcccaaactaatct |
| | | aggaatcactgtctaaccactc |
| | | actgtcatgaatgtgtacttaa |
| | | aggatgaggttgagtcatacca |
| | | aatagtgatttcgatagttcaa |
| | | aatggtgaaattagcaattcta |
| | | catgattcagtctaatcaatgg |
| | | ataccgactgtttcccacacaa |
| | | gtctcctgttctcttaagctta |
| | | ctcactgacagcctttcactct |
| | | ccacaaatacattaaagatatg |
| | | gccatcaccaagccccctagga |
| | | tgacaccagacctgagagtctg |
| | | aagacctggatccaagttctga |
| | | cttttccccctgacagctgtgt |
| | | gaccttcgtgaagtcgccaaac |
| | | ctctctgagccccagtcattgc |
| | | tagtaagacctgcctttgagtt |
| | | ggtatgatgttcaagttagata |
| | | acaaaatgtttatacccattag |
| | | aacagagaataaatagaactac |
| | | atttcttgca |
| 70 | Wild Type | cctttcccagggacttctacaa |
| | Human Serum | ggaaaaagctagagttggttac |
| | Albumin | tgacttctaataaataatgcct |
| | Nucleic Acid | acaatttctaggaagttaaaag |
| | | ttgacataatttatccaagaaa |
| | | gaattattttcttaacttagaa |
| | | tagtttcttttttcttttcaga |
| | | tgtaggtttttctggctttaga |
| | | aaaaatgcttgtttttcttcaa |
| | | tggaaaataggcacacttgttt |
| | | tatgtctgttcatctgtagtca |
| | | gaaagacaagtctggtatttcc |
| | | tttcaggactcccttgagtcat |
| | | taaaaaaaatcttcctatctat |
| | | ctatgtatctatcatccatcta |
| | | gctttgattttttcctcttctg |
| | | tgctttattagttaattagtac |
| | | ccatttctgaagaagaaataac |
| | | ataagattatagaaaataattt |
| | | ctttcattgtaagactgaatag |
| | | aaaaaattttctttcattataa |
| | | gactgagtagaaaaaataatac |
| | | tttgttagtctctgtgcctcta |
| | | tgtgccatgaggaaatttgact |
| | | actggttttgactgactgagtt |
| | | atttaattaagtaaaataactg |
| | | gcttagtactaattattgttct |
| | | gtagtatcagagaaagttgttc |
| | | ttcctactggttgagctcagta |
| | | gttcttcatattctgagcaaaa |
| | | gggcagaggtaggatagctttt |
| | | ctgaggtagagataagaacctt |
| | | gggtagggaaggaagatttatg |
| | | aaatatttaaaaaattattctt |
| | | ccttcgctttgtttttagacat |
| | | aatgttaaatttattttgaaat |
| | | ttaaagcaacataaaagaacat |
| | | gtgatttttctacttattgaaa |
| | | gagagaaaggaaaaaaatatga |
| | | aacagggatggaaagaatccta |
| | | tgcctggtgaaggtcaagggtt |
| | | ctcataacctacagagaatttg |
| | | gggtcagcctgtcctattgtat |
| | | attatggcaaagataatcatca |
| | | tctcatttgggtccattttcct |
| | | ctccatctctgcttaactgaag |
| | | atcccatgagatatactcacac |
| | | tgaatctaaatagcctatctca |
| | | gggcttgaatcacatgtgggcc |
| | | acagcaggaatgggaacatgga |
| | | atttctaagtcctatcttactt |
| | | gttattgttgctatgtcttttt |
| | | cttagtttgcatctgaggcaac |
| | | atcagctttttcagacagaatg |
| | | gctttggaatagtaaaaaagac |
| | | acagaagccctaaaatatgtat |
| | | gtatgtatatgtgtgtgtgcat |
| | | gcgtgagtacttgtgtgtaaat |
| | | ttttcattatctataggtaaaa |
| | | gcacacttggaattagcaatag |
| | | atgcaatttgggacttaactct |
| | | ttcagtatgtcttatttctaag |
| | | caaagtatttagtttggttagt |
| | | aattactaaacactgagaacta |
| | | aattgcaaacaccaagaactaa |
| | | aatgttcaagtgggaaattaca |
| | | gttaaataccatggtaatgaat |
| | | aaaaggtacaaatcgtttaaac |
| | | tcttatgtaaaatttgataaga |
| | | tgttttacacaactttaataca |
| | | ttgacaaggtcttgtggagaaa |
| | | acagttccagatggtaaatata |
| | | cacaagggatttagtcaaacaa |
| | | ttttttggcaagaatattatga |
| | | attttgtaatcggttggcagcc |
| | | aatgaaatacaaagatgagtct |
| | | agttaataatctacaattattg |
| | | gttaaagaagtatattagtgct |
| | | aatttccctccgtttgtcctag |
| | | cttttctcttctgtcaacccca |
| | | cacgcctttggcacaatgaagt |
| | | gggtaacctttatttcccttct |
| | | ttttctctttagctcggcttat |
| | | tccaggggtgtgtttcgtcgag |
| | | atgcacgtaagaaatccatttt |
| | | tctattgttcaacttttattct |
| | | attttcccagtaaaataaagtt |
| | | ttagtaaactctgcatctttaa |
| | | agaattattttggcatttattt |
| | | ctaaaatggcatagtattttgt |
| | | atttgtgaagtcttacaaggtt |
| | | atcttattaataaaattcaaac |
| | | atcctaggtaaaaaaaaaaaaa |
| | | ggtcagaattgtttagtgactg |
| | | taattttcttttgcgcactaag |
| | | gaaagtgcaaagtaacttagag |
| | | tgactgaaacttcacagaatag |
| | | ggttgaagattgaattcataac |
| | | tatcccaaagacctatccattg |
| | | cactatgctttatttaaaaacc |
| | | acaaaacctgtgctgttgatct |
| | | cataaatagaacttgtatttat |
| | | atttattttcattttagtctgt |
| | | cttcttggttgctgttgataga |
| | | cactaaaagagtattagatatt |
| | | atctaagtttgaatataaggct |
| | | ataaatatttaataatttttaa |
| | | aatagtattcttggtaattgaa |
| | | ttattcttctgtttaaaggcag |
| | | aagaaataattgaacatcatcc |
| | | tgagtttttctgtaggaatcag |
| | | agcccaatattttgaaacaaat |
| | | gcataatctaagtcaaatggaa |
| | | agaaatataaaaagtaacatta |
| | | ttacttcttgttttcttcagta |
| | | tttaacaatccttttttttctt |
| | | cccttgcccagacaagagtgag |
| | | gttgctcatcggtttaaagatt |
| | | tgggagaagaaaatttcaaagc |
| | | cttgtaagttaaaatattgatg |
| | | aatcaaatttaatgtttctaat |
| | | agtgttgtttattattctaaag |
| | | tgcttatatttccttgtcatca |
| | | gggttcagattctaaaacagtg |
| | | ctgcctcgtagagttttctgcg |
| | | ttgaggaagatattctgtatct |
| | | gggctatccaataaggtagtca |
| | | ctggtcacatggctattgagta |
| | | cttcaaatatgacaagtgcaac |
| | | tgagaaacaaaaacttaaattg |
| | | tatttaattgtagttaatttga |
| | | atgtatatagtcacatgtggct |
| | | aatggctactgtattggacagt |
| | | acagctctggaacttgcttggt |
| | | ggaaaggactttaatataggtt |
| | | tcctttggtggcttacccacta |
| | | aatcttctttacatagcaagca |
| | | ttcctgtgcttagttgggaata |
| | | tttaatttttttttttttttaa |
| | | gacagggtctcgctctgtcgcc |
| | | caggctggagtgcagtggcgca |
| | | atctcggctcactgcaaactcc |
| | | gctcccgggttcacgccattct |
| | | cctgcctcagcctcccgagtag |
| | | ctgggactacaggcgcccgcca |
| | | tcacgcccggctaatcttttgt |
| | | atttttagtagagatggggttt |
| | | caccgtgtgccaggatggtctc |
| | | aatctcctgacatcgtgatctg |
| | | cccacctcggcctcccaaagtg |
| | | ctgggattacaggagtgagtca |
| | | ccgcgcccggcctatttaaatg |
| | | ttttttaatctagtaaaaaatg |
| | | agaaaattgtttttttaaaagt |
| | | ctacctaatcctacaggctaat |
| | | taaagacgtgtgtggggatcag |
| | | gtgcggtggttcacacctgtaa |
| | | tcccagcactttggaaggctga |
| | | tgcaggaggattgcttgagccc |
| | | aggagtacaagaccagcctggg |
| | | caagtctctttaaaaaaaacaa |
| | | aacaaacaaacaaaaaaattag |
| | | gcatggtggcacatgcctgtag |
| | | tcctagctacttaggaggctga |
| | | cgtaggaggatcgtttggacct |
| | | gagaggtcaaggctacagtgag |
| | | ccatgattgtgccactgcactc |
| | | cagcctgggtgacagagtgaga |
| | | ctctgtctcaaaaaagaaaaag |
| | | gaaatctgtggggtttgtttta |
| | | gttttaagtaattctaaggact |
| | | ttaaaaatgcctagtcttgaca |
| | | attagatctatttggcatacaa |
| | | tttgcttgcttaatctatgtgt |
| | | gtgcatagatctactgacacac |
| | | gcatacatataaacattaggga |
| | | actaccattctctttgcgtagg |
| | | aagccacatatgcctatctagg |
| | | cctcagatcatacctgatatga |
| | | ataggctttctggataatggtg |
| | | aagaagatgtataaaagataga |
| | | acctatacccatacatgatttg |
| | | ttctctagcgtagcaacctgtt |
| | | acatattaaagttttattatac |
| | | tacatttttctacatcctttgt |
| | | ttcagggtgttgattgcctttg |
| | | ctcagtatcttcagcagtgtcc |
| | | atttgaagatcatgtaaaatta |
| | | gtgaatgaagtaactgaatttg |
| | | caaaaacatgtgttgctgatga |
| | | gtcagctgaaaattgtgacaaa |
| | | tcacttgtaagtacattctaat |
| | | tgtggagattctttcttctgtt |
| | | tgaagtaatcccaagcatttca |
| | | aaggaattttttttaagttttc |
| | | tcaattattattaagtgtcctg |
| | | atttgtaagaaacactaaaaag |
| | | ttgctcatagactgataagcca |
| | | ttgtttcttttgtgatagagat |
| | | gctttagctatgtccacagttt |
| | | taaaatcatttctttattgaga |
| | | ccaaacacaacagtcatggtgt |
| | | atttaaatggcaatttgtcatt |
| | | tataaacacctctttttaaaat |
| | | ttgaggtttggtttctttttgt |
| | | agaggctaatagggatatgata |
| | | gcatgtatttatttatttattt |
| | | atcttattttattatagtaaga |
| | | acccttaacatgagatctaccc |
| | | tgttatatttttaagtgtacaa |
| | | tccattattgttaactacgggt |
| | | acactgttgtatagcttactca |
| | | tcttgctgtattaaaactttgt |
| | | gcccattgattagtaacccctc |
| | | gtttcgtcctcccccagccact |
| | | ggcaaccagcattatactcttt |
| | | gattctatgagtttgactactt |
| | | tagctaccttatataagtggta |
| | | ttatgtactgtttatcttttta |
| | | tgactgacttatttcccttagc |
| | | atagtgcattcaaagtccaacc |
| | | atgttgttgcctattgcagaat |
| | | ttccttcttttcaaggctgaat |
| | | aatattccagtgcatgtgtgta |
| | | ccacattttctttatccattaa |
| | | tttgttgattgatagacattta |
| | | ggttggttttctacatcttgac |
| | | tatcatgaatagtgttgcaatg |
| | | aacacaggagagctactatctc |
| | | ttagagatgatatcatggtttt |
| | | tatcatcagaaaacacccactg |
| | | atttctatgctaattttgttac |
| | | ctgggtggaataatagtacagc |
| | | tatatattcctcattttagata |
| | | tctttgtatttctacatacaat |
| | | aaaaaagcagagtacttagtca |
| | | tgttgaagaactttaaactttt |
| | | agtatttccagatcaatcttca |
| | | aaacaaggacaggtttatcttt |
| | | ctctcaccactcaatctatata |
| | | tacctcttgtgggcaaggccag |
| | | tttttatcactggagcctttcc |
| | | cctttttattatgtacctctcc |
| | | ctcacagcagagtcaggacttt |
| | | aactttacacaatactatggct |
| | | ctacatatgaaatcttaaaaat |
| | | acataaaaattaataaattctg |
| | | tctagagtagtatattttccct |
| | | ggggttacgattactttcataa |
| | | taaaaattagagataaggaaag |
| | | gactcatttattggaaagtgat |
| | | tttaggtaacatttctggaaga |
| | | aaaatgtctatatcttaatagt |
| | | cacttaatatatgatggattgt |
| | | gttactcctcagttttcaatgg |
| | | catatactaaaacatggccctc |
| | | taaaaagggggcaaatgaaatg |
| | | agaaactctctgaatgtttttc |
| | | tcccctaggtgaattcacctgc |
| | | tgcttagaagcttattttctct |
| | | tgatttctgttataatgattgc |
| | | tcttaccctttagttttaagtt |
| | | tcaaaataggagtcatataact |
| | | ttccttaaagctattgactgtc |
| | | tttttgtcctgttttattcacc |
| | | atgagttatagtgtgacagtta |
| | | attcttatgaaaattatataga |
| | | gatggttaaatcatcagaaact |
| | | gtaaacctcgattgggagggga |
| | | agcggatttttaaatgatttcc |
| | | tgaccaagcttaaccagtatat |
| | | taaatcctttgtactgttcttt |
| | | ggctataaagaaaaaaggtact |
| | | gtccagcaactgaaacctgctt |
| | | tcttccatttagcatacccttt |
| | | ttggagacaaattatgcacagt |
| | | tgcaactcttcgtgaaacctat |
| | | ggtgaaatggctgactgctgtg |
| | | caaaacaagaacctgagagaaa |
| | | tgaatgcttcttgcaacacaaa |
| | | gatgacaacccaaacctccccc |
| | | gattggtgagaccagaggttga |
| | | tgtgatgtgcactgcttttcat |
| | | gacaatgaagagacatttttga |
| | | aaaagtaagtaatcagatgttt |
| | | atagttcaaaattaaaaagcat |
| | | ggagtaactccataggccaaca |
| | | ctctataaaaattaccataaca |
| | | aaaatattttcaacattaagac |
| | | ttggaagttttgttatgatgat |
| | | tttttaaagaagtagtatttga |
| | | taccacaaaattctacacagca |
| | | aaaaatatgatcaaagatattt |
| | | tgaagtttattgaaacaggata |
| | | caatctttctgaaaaatttaag |
| | | atagacaaattatttaatgtat |
| | | tacgaagatatgtatatatggt |
| | | tgttataattgatttcgtttta |
| | | gtcagcaacattatattgccaa |
| | | aatttaaccatttatgcacaca |
| | | cacacacacacacacacactta |
| | | acccttttttccacatacttaa |
| | | agaatgacagagacaagaccat |
| | | catgtgcaaattgagcttaatt |
| | | ggttaattagatatctttggaa |
| | | tttggaggttctggggagaatg |
| | | tcgattacaattatttctgtaa |
| | | tattgtctgctatagaaaagtg |
| | | actgtttttctttttcaaaatt |
| | | tagatacttatatgaaattgcc |
| | | agaagacatccttacttttatg |
| | | ccccggaactccttttctttgc |
| | | taaaaggtataaagctgctttt |
| | | acagaatgttgccaagctgctg |
| | | ataaagctgcctgcctgttgcc |
| | | aaaggtattatgcaaaagaata |
| | | gaaaaaaagagtteattatcca |
| | | acctgattttgtccattttgtg |
| | | gctagatttagggaacctgagt |
| | | gtctgatacaaactttccgaca |
| | | tggtcaaaaaagccttcctttt |
| | | atctgtcttgaaaatctttcat |
| | | ctttgaaggcctacactctcgt |
| | | ttcttcttttaagatttgccaa |
| | | tgatgatctgtcagaggtaatc |
| | | actgtgcatgtgtttaaagatt |
| | | tcaccactttttatggtggtga |
| | | tcactatagtgaaatactgaaa |
| | | cttgtttgtcaaattgcacagc |
| | | aaggggacacagttcttgttta |
| | | tcttttcatgataatttttagt |
| | | agggagggaattcaaagtagag |
| | | aattttactgcatctagatgcc |
| | | tgagttcatgcattcattccat |
| | | aaatatatattatggaatgctt |
| | | tattttcttttctgaggagttt |
| | | actgatgttggtggaggagaga |
| | | ctgaaatgaattatacacaaaa |
| | | tttaaaaattagcaaaattgca |
| | | gcccctgggatattagcgtact |
| | | ctttctctgacttttctcccac |
| | | ttttaaggctctttttcctggc |
| | | aatgtttccagttggtttctaa |
| | | ctacatagggaattccgctgtg |
| | | accagaatgatcgaatgatctt |
| | | tccttttcttagagagcaaaat |
| | | cattattcgctaaagggagtac |
| | | ttgggaatttaggcataaatta |
| | | tgccttcaaaatttaatttggc |
| | | acagtctcatctgagcttatgg |
| | | aggggtgtttcatgtagaattt |
| | | ttcttctaattttcatcaaatt |
| | | attcctttttgtagctcgatga |
| | | acttcgggatgaagggaaggct |
| | | tcgtctgccaaacagagactca |
| | | agtgtgccagtctccaaaaatt |
| | | tggagaaagagctttcaaagca |
| | | tggtaaatacttttaaacatag |
| | | ttggcatctttataacgatgta |
| | | aatgataatgcttcagtgacaa |
| | | attgtacatttttatgtatttt |
| | | gcaaagtgctgtcaaatacatt |
| | | tctttggttgtctaacaggtag |
| | | aactctaatagaggtaaaaatc |
| | | agaatatcaatgacaatttgac |
| | | attatttttaatcttttctttt |
| | | ctaaatagttgaataatttaga |
| | | ggacgctgtcctttttgtccta |
| | | aaaaaagggacagatatttaag |
| | | ttctatttatttataaaatctt |
| | | ggactcttattctaatggttca |
| | | ttatttttatagagctgtaggc |
| | | atggttctttatttaatttttt |
| | | aaagttatttttaatttttgtg |
| | | gatacagagtaggtatacatat |
| | | ttacggggtatatgagatattt |
| | | tgatataagtatacaacatata |
| | | taatccctttatttaattttat |
| | | cttccccccaatgatctaaaac |
| | | tatttgcttgtccttttatgtc |
| | | ttatagttaaattcagtcacca |
| | | actaagttgaagttacttctta |
| | | tttttgcatagctccagctctg |
| | | atcttcatctcatgtttttgcc |
| | | tgagcctctgttttcatattac |
| | | ttagttggttctgggagcatac |
| | | tttaatagccgagtcaagaaaa |
| | | atactagctgccccgtcaccca |
| | | cactcctcacctgctagtcaac |
| | | agcaaatcaacacaacaggaaa |
| | | taaaatgaaaataatagacatt |
| | | atgcatgctctctagaaactgt |
| | | caattgaactgtatttgctcat |
| | | cattcctaccatctacaccacc |
| | | aaaatcaaccaaatttatgaaa |
| | | aaaaaacagccccaacataaaa |
| | | ttatacacagataaacaggcta |
| | | tgattggttttgggaaagaagt |
| | | cacctttacctgatttaggcaa |
| | | ctgtgaaatgactagagaatga |
| | | agaaaattagacgtttacatct |
| | | tgtcatagagtttgaagatagt |
| | | gctggatctttctttttataag |
| | | taagatcaataaaaactccctc |
| | | attctgtagaagttatgatttc |
| | | ttttctaagagacctttagaag |
| | | tcagaaaaaatgtgtttcaatt |
| | | gagaaaaaagataactggagtt |
| | | tgtgtagtacttcccagattat |
| | | aaaatgcttttgtatgtattat |
| | | ctaatttaatcctcaaaacttc |
| | | ttcaatttagcatgttgtcatg |
| | | acactgcagaggctgaagctca |
| | | gagacgctgagccctctgctaa |
| | | caagtcctactgctaacaagtg |
| | | ataaagccagagctggaagtca |
| | | catctggactccaaacctgatg |
| | | cttctcagcctgttgccccttt |
| | | tagagttcctttttaatttctg |
| | | cttttatgacttgctagatttc |
| | | tacctaccacacacactcttaa |
| | | atggataattctgccctaagga |
| | | taagtgattaccatttggttca |
| | | gaactagaactaatgaatttta |
| | | aaaattatttctgtatgtccat |
| | | tttgaattttcttatgagaaat |
| | | agtatttgcctagtgttttcat |
| | | ataaaatatcgcatgataatac |
| | | cattttgattggcgattttctt |
| | | tttagggcagtagctcgcctga |
| | | gccagagatttcccaaagctga |
| | | gtttgcagaagtttccaagtta |
| | | gtgacagatcttaccaaagtcc |
| | | acacggaatgctgccatggaga |
| | | tctgcttgaatgtgctgatgac |
| | | agggtaaagagtcgtcgatatg |
| | | ctttttggtagcttgcatgctc |
| | | aagttggtagaatggatgcgtt |
| | | tggtatcattggtgatagctga |
| | | cagtgggttgagattgtcttct |
| | | gtgctttcgtctgtcctatctt |
| | | caatctttccctgcctatggtg |
| | | gtggtacctttctgtttttaac |
| | | ctgctataaattaccagataaa |
| | | cccattcactgatttgtaactc |
| | | ctttcagtcatgctctaactgt |
| | | aaatgaaggcttaaactgaagt |
| | | agaacagttacaaggttttact |
| | | tggcagaacatcttgcaaggta |
| | | gatgtctaagaagatttttttt |
| | | tctttttttaagacagagtttc |
| | | gctcttgtttcccaggctgggg |
| | | tgcaatggtgtgatcttggctc |
| | | agcgcaacctctgcctcctggg |
| | | ttcaagtgattttcatgcctca |
| | | gcctcccaagtagctgggatta |
| | | caggcatgcgccaccacacctg |
| | | gctaattttgtatttttagtag |
| | | aggcggggtttcaccatattgt |
| | | ccagactggtctcgaactcctg |
| | | acctcaggtgatccacccgcct |
| | | tggcctcccaaagtgctgggat |
| | | tacaggcatgagccaccttgcc |
| | | cagcctaagaagattttttgag |
| | | ggaggtaggtggacttggagaa |
| | | ggtcactacttgaagagatttt |
| | | tggaaatgatgtatttttcttc |
| | | tctatattccttcccttaatta |
| | | actctgtttgttagatgtgcaa |
| | | atatttggaatgatatctcttt |
| | | tctcaaaacttataatattttc |
| | | tttctccctttcttcaagatta |
| | | aacttatgggcaaatactagaa |
| | | tcctaatctctcatggcacttt |
| | | ctggaaaatttaaggcggttat |
| | | tttatatatgtaagcagggcct |
| | | atgactatgatcttgactcatt |
| | | tttcaaaaatcttctatatttt |
| | | atttagttatttggtttcaaaa |
| | | ggcctgcacttaattttggggg |
| | | attatttggaaaaacagcattg |
| | | agttttaatgaaaaaaacttaa |
| | | atgccctaacagtagaaacata |
| | | aaattaataaataactgagctg |
| | | agcacctgctactgattagtct |
| | | attttaattaagtgggaatgtt |
| | | tttgtagtcctatctacatctc |
| | | caggtttaggagcaaacagagt |
| | | atgttcatagaaggaatatgtg |
| | | tatggtcttagaatacaatgaa |
| | | catgttctgccaacttaataaa |
| | | ggtctgaggagaaagtgtagca |
| | | atgtcaattcgtgttgaacaat |
| | | ttccaccaacttacttataggc |
| | | ggaccttgccaagtatatctgt |
| | | gaaaatcaagattcgatctcca |
| | | gtaaactgaaggaatgctgtga |
| | | aaaacctctgttggaaaaatcc |
| | | cactgcattgccgaagtggaaa |
| | | atgatgagatgcctgctgactt |
| | | gccttcattagctgctgatttt |
| | | gttgaaagtaaggatgtttgca |
| | | aaaactatgctgaggcaaagga |
| | | tgtcttcctgggcatgtaagta |
| | | gataagaaattattcttttata |
| | | gctttggcatgacctcacaact |
| | | taggaggatagcctaggctttt |
| | | ctgtggagttgctacaatttcc |
| | | ctgctgcccagaatgtttcttc |
| | | atccttccctttcccaggcttt |
| | | aacaatttttgaaatagttaat |
| | | tagttgaatacattgtcataaa |
| | | ataatacatgttcacggcaaag |
| | | ctcaacattccttactccttag |
| | | gggtatttctgaaaatacgtct |
| | | agaaacattttgtgtatatata |
| | | aattatgtatacttcagtcatt |
| | | cattccaagtgtatttcttgaa |
| | | catctataatatatgtgtgtga |
| | | ctatgtattgcctgtctatcta |
| | | actaatctaatctaatctagtc |
| | | tatctatctaatctatgcaatg |
| | | atagcaaagaagtataaaaaga |
| | | aatatagagtctgacacaggtg |
| | | ctttatatttggtgaaaagacc |
| | | agaagttcagtataatggcaat |
| | | atggtaggcaactcaattacaa |
| | | aataaatgtttacgtattgtca |
| | | gaagttgtggtgataaactgca |
| | | tttttgttgttggattatgata |
| | | atgcactaaataatatttccta |
| | | aaattatgtaccctacaagatt |
| | | tcactcatacagagaagaaaga |
| | | gaatattttaagaacatatctc |
| | | tgcccatctatttatcagaatc |
| | | cttttgagatgtagtttaaatc |
| | | aaacaaaatgttaataaaaata |
| | | acaagtatcattcatcaaagac |
| | | ttcatatgtgccaagcagtgtg |
| | | tgctttgtgtagattatgtcat |
| | | atagttctcataatccaccttc |
| | | cgagacagatactatttatttt |
| | | ttgagacagagttttactcttg |
| | | ttgcccaggctggagtgcaatg |
| | | gtgccatctcggctcaccacaa |
| | | ccttcgcctcccaggttcaagc |
| | | gattctcctgcctcagcctcct |
| | | gggattacaggcatgcaccacc |
| | | atgcctggctaattttgtattt |
| | | ttagtagagatggggtttcacc |
| | | atgttggtcagactggtctcaa |
| | | actcctgacctctggtgatatg |
| | | cctgcctcagcctcctaaagtg |
| | | ctgggattacaggcatgagcca |
| | | ctgtgcccagccgacagatact |
| | | attattatttccattctaccga |
| | | gaaggagactaaggctctgatc |
| | | atttaaataagttgcctaaggt |
| | | gatgcagtgatataagtagcag |
| | | agctaggaattgagccttggta |
| | | actttaactctggaccccaagt |
| | | ccttagctactaagctttactg |
| | | catggggtttagtcaaattaag |
| | | acttttggaatatgagttactt |
| | | ttgagattagctttgtgatatt |
| | | ttttgtgctcatttgtccaaca |
| | | aagtctattttattttcatctt |
| | | aattaggtttttgtatgaatat |
| | | gcaagaaggcatcctgattact |
| | | ctgtcgtgctgctgctgagact |
| | | tgccaagacatatgaaaccact |
| | | ctagagaagtgctgtgccgctg |
| | | cagatcctcatgaatgctatgc |
| | | caaagtggtaggtttattgttg |
| | | gaaaaaaatgtagttctttgac |
| | | tgatgattccaataatgagaaa |
| | | gaaaaataatgcaagaatgtaa |
| | | aatgatatacagtgcaatttag |
| | | atcttttcttgagatggtttca |
| | | attctggaatcttaaacatgaa |
| | | agaaaaagtagccttagaatga |
| | | ttaacaaaatttagactagtta |
| | | gaatagaaagatctgaatagag |
| | | caatctctaaaaaattttgatc |
| | | tttttttctctttttcacaatc |
| | | ctgagaacaaaaaaaaattaaa |
| | | tttaaatgttaattagaagata |
| | | tttaacttagatgtaaagtgag |
| | | ttaacctgattccaggattaat |
| | | caagtactagaattagtatctt |
| | | atggcaaattatagaacctatc |
| | | cctttagaatattttcaaatct |
| | | ttttgaggatgtttaggaatag |
| | | ttttacaagaaattaagttagg |
| | | agaggaaatctgttctggagga |
| | | tttttagggttcccactagcat |
| | | atgtaatggtttctgaactatt |
| | | cagaatcagagaaaactcattt |
| | | ttcctgctttcaagaagctact |
| | | gtatgccaggcaccatgcacaa |
| | | acaatgaccaacgtaaaatctc |
| | | tcattttggagagcctggaatc |
| | | taactggaaaggtgaactaata |
| | | ataataatatgtacaatcatag |
| | | ccatcatttattaaacttttat |
| | | tatatgcaaggcactgtttaat |
| | | ttcattagcttacctggtttac |
| | | agagcagctctatgagatgagt |
| | | gccatctttgcccctattttag |
| | | ggataaggattccgaaatgtgg |
| | | agatggtaagtaaaattgcaca |
| | | actgaagaatgagttacatgac |
| | | ttggctcaaatactggtcattg |
| | | aactccagagcctgaatattct |
| | | taaccacttacatgatgcaagc |
| | | tcaccaaataaatagttcgaat |
| | | gtattgtgacagagcggcattg |
| | | atattcatctattcatgtggct |
| | | ttgagtaggaagaagaaaggat |
| | | atcattctgaccagaggggtga |
| | | aaaacaacctgcatctgatcct |
| | | gaggcataatactattaacaca |
| | | attcttttatgtttcagttcga |
| | | tgaatttaaacctcttgtggaa |
| | | gagcctcagaatttaatcaaac |
| | | aaaattgtgagctttttgagca |
| | | gcttggagagtacaaattccag |
| | | aatgcgtaagtaatttttattg |
| | | actgattttttttatcaatttg |
| | | taattatttaagacttaatata |
| | | tgagccacctagcatagaactt |
| | | ttaagaatgaaaatacattgca |
| | | tatttctaatcactctttgtca |
| | | agaaagataggagaggagagat |
| | | aaaatagttgatggggtggaga |
| | | ggtctatatttgaatgtagtct |
| | | aaaaattgttctcttaagattg |
| | | gaagtatgtaggctgggagggt |
| | | aaataccaaatcttggtatatc |
| | | agaactgagcatgtcccttgaa |
| | | ggttaagaaatagttaatgggc |
| | | aaatagagcatggcaatatttt |
| | | gtagagcagcaagtagtaggcc |
| | | ttgaatagatgtcgctcaaaaa |
| | | gtaatatgtaagctgaacacaa |
| | | aaatgtaacaaatgaatttaga |
| | | tacatatttgaatattaaattc |
| | | aggttgtttgggagatgcacct |
| | | agtctttgatggttaaaccttt |
| | | ccctccatagaagagacagaga |
| | | cagaatggcttgctggactaat |
| | | gtcccaattcaatagagtctta |
| | | tctacgaaggttaaaaacaaga |
| | | agagacatattatacagtagat |
| | | atttattgtgtggctcatacac |
| | | atggtgctcttctgattatgga |
| | | ttttagagataataacagtgaa |
| | | caagacatagtttctttcctcg |
| | | agtagattaaagtcatacattg |
| | | acttttaatggtgactggcatt |
| | | cttaatacatgattattatata |
| | | ttaggtaccatgtcagattaat |
| | | tataatactttactatttttaa |
| | | tttaacccttgaactatcccta |
| | | ttgagtcagatatatttccttc |
| | | cattttctacttgtatctttea |
| | | agtttagcatatgctgatacat |
| | | atgaagctctctccaggtttta |
| | | ttgaaagaagaaattaataaat |
| | | ttattaatgtcactgaattagg |
| | | caactcactttcccaagattat |
| | | gcaagtggtacaggtggaactc |
| | | aaagccaagtttaactagttgt |
| | | tcaggagaatgttttctaccct |
| | | ccactaacccactactctgcag |
| | | atggagataatatgatgaatgg |
| | | aacatagcaacatcttagttga |
| | | ttccggccaagtgttctctgtt |
| | | ttatctactatgttagacagtt |
| | | tcttgccttgctgaaaacacat |
| | | gacttctttttttcaggctatt |
| | | agttcgttacaccaagaaagta |
| | | ccccaagtgtcaactccaactc |
| | | ttgtagaggtctcaagaaacct |
| | | aggaaaagtgggcagcaaatgt |
| | | tgtaaacatcctgaagcaaaaa |
| | | gaatgccctgtgcagaagacta |
| | | tgtgagtctttaaaaaaatata |
| | | ataaattaataatgaaaaaatt |
| | | ttacctttagatattgataatg |
| | | ctagctttcataagcagaagga |
| | | agtaatgtgtgtgtgtgcatgt |
| | | ttgtgtgcatgtgtgtgtgcat |
| | | gcacgtgtgtgtatgtgtgata |
| | | ttggcagtcaaggccccgagga |
| | | tgataatttttttttttttttt |
| | | gagacggagtctcgctttgttg |
| | | tccaggctggagtgcagtggtg |
| | | ccatctcggctcactgcaacct |
| | | ccgcctcccaagttcaagccat |
| | | tctcctgcctcagcctcccaag |
| | | tagctgggactacaggtgcatg |
| | | ccaccatgcctggctaattttt |
| | | tgtatttttagtagaaaatttt |
| | | cagcttcacctcttttgaattt |
| | | ctgctctcctgcctgttcttta |
| | | gctatccgtggtcctgaaccag |
| | | ttatgtgtgttgcatgagaaaa |
| | | cgccagtaagtgacagagtcac |
| | | caaatgctgcacagaatccttg |
| | | gtgaacaggcgaccatgctttt |
| | | cagctctggaagtcgatgaaac |
| | | atacgttcccaaagagtttaat |
| | | gctgaaacattcaccttccatg |
| | | cagatatatgcacactttctga |
| | | gaaggagagacaaatcaagaaa |
| | | caaacgtgaggagtatttcatt |
| | | actgcatgtgtttgtagtcttg |
| | | atagcaagaactgtcaattcaa |
| | | gctagcaactttttcctgaagt |
| | | agtgattatatttcttagagga |
| | | aagtattggagtgttgccctta |
| | | ttatgctgataagagtacccag |
| | | aataaaatgaataactttttaa |
| | | agacaaaatcctctgttataat |
| | | attgctaaaattattcagagta |
| | | atattgtggattaaagccacaa |
| | | tagaataacatgttagaccata |
| | | ttcagtagaaaaagatgaacaa |
| | | ttaactgataaatttgtgcaca |
| | | tggcaaattagttaatgggaac |
| | | cataggagaatttatttctaga |
| | | tgtaaataattattttaagttt |
| | | gccctatggtggccccacacat |
| | | gagacaaacccccaagatgtga |
| | | cttttgagaatgagacttggat |
| | | aaaaaacatgtagaaatgcaag |
| | | ccctgaagctcaactccctatt |
| | | gctatcacaggggttataattg |
| | | cataaaatttagctatagaaag |
| | | ttgctgtcatctcttgtgggct |
| | | gtaatcatcgtctaggcttaag |
| | | agtaatattgcaaaacctgtca |
| | | tgcccacacaaatctctccctg |
| | | gcattgttgtctttgcagatgt |
| | | cagtgaaagagaaccagcagct |
| | | cccatgagtttggatagcctta |
| | | ttttctatagcctccccactga |
| | | agggagcaaagtttaagaacca |
| | | aatataaagtttctcatcttta |
| | | tagatgagaaaaattttaaata |
| | | aagtccaagataattaaatttt |
| | | taaggatcatttttagctcttt |
| | | aatagcaataaaactcaatatg |
| | | acataatatggcacttccaaaa |
| | | tctgaataatatataattgcaa |
| | | tgacatacttcttttcagagat |
| | | ttactgaaaagaaatttgttga |
| | | cactacataacgtgatgagtgg |
| | | tttatactgattgtttcagttg |
| | | gtcttcccaccaactccatgaa |
| | | agtggattttattatcctcatc |
| | | atgcagatgagaatattgagac |
| | | ttatagcggtatgcctggccca |
| | | agtactcagagttgcctggctc |
| | | caagatttataatcttaaatga |
| | | tgggactaccatccttactctc |
| | | tccatttttctatacgtgagta |
| | | atgttttttctgtttttttttt |
| | | ttctttttccattcaaactcag |
| | | tgcacttgttgagctcgtgaaa |
| | | cacaagcccaaggcaacaaaag |
| | | agcaactgaaagctgttatgga |
| | | tgatttcgcagcttttgtagag |
| | | aagtgctgcaaggctgacgata |
| | | aggagacctgctttgccgagga |
| | | ggtactacagttctcttcattt |
| | | taatatgtccagtattcatttt |
| | | tgcatgtttggttaggctaggg |
| | | cttagggatttatatatcaaag |
| | | gaggctttgtacatgtgggaca |
| | | gggatcttattttacaaacaat |
| | | tgtcttacaaaatgaataaaac |
| | | agcactttgtttttatctcctg |
| | | ctctattgtgccatactgttga |
| | | atgtttataatgcatgttctgt |
| | | ttccaaatttgtgatgcttatg |
| | | aatattaataggaatatttgta |
| | | aggcctgaaatattttgatcat |
| | | gaaatcaaaacattaatttatt |
| | | taaacatttacttgaaatgtgg |
| | | tggtttgtgatttagttgattt |
| | | tataggctagtgggagaattta |
| | | cattcaaatgtctaaatcactt |
| | | aaaatttccctttatggcctga |
| | | cagtaacttttttttattcatt |
| | | tggggacaactatgtccgtgag |
| | | cttccatccagagattatagta |
| | | gtaaattgtaattaaaggatat |
| | | gatgcacgtgaaatcactttgc |
| | | aatcatcaatagcttcataaat |
| | | gttaattttgtatcctaatagt |
| | | aatgctaatattttcctaacat |
| | | ctgtcatgtctttgtgttcagg |
| | | gtaaaaaacttgttgctgcaag |
| | | tcaagctgccttaggcttataa |
| | | catcacatttaaaagcatctca |
| | | ggtaactatattttgaattttt |
| | | taaaaaagtaactataatagtt |
| | | attattaaaatagcaaagattg |
| | | accatttccaagagccatatag |
| | | accagcaccgaccactattcta |
| | | aactatttatgtatgtaaatat |
| | | tagcttttaaaattctcaaaat |
| | | agttgctgagttgggaaccact |
| | | attatttctattttgtagatga |
| | | gaaaatgaagataaacatcaaa |
| | | gcatagattaagtaattttcca |
| | | aagggtcaaaattcaaaattga |
| | | aaccaaggtttcagtgttgccc |
| | | attgtcctgttctgacttatat |
| | | gatgcggtacacagagccatcc |
| | | aagtaagtgatggctcagcagt |
| | | ggaatactctgggaattaggct |
| | | gaaccacatgaaagagtgcttt |
| | | atagggcaaaaacagttgaata |
| | | tcagtgatttcacatggttcaa |
| | | cctaatagttcaactcatcctt |
| | | tccattggagaatatgatggat |
| | | ctaccttctgtgaactttatag |
| | | tgaagaatctgctattacattt |
| | | ccaatttgtcaacatgctgagc |
| | | tttaataggacttatcttctta |
| | | tgacaacatttattggtgtgtc |
| | | cccttgcctagcccaacagaag |
| | | aattcagcagccgtaagtctag |
| | | gacaggcttaaattgttttcac |
| | | tggtgtaaattgcagaaagatg |
| | | atctaagtaatttggcatttat |
| | | tttaataggtttgaaaaacaca |
| | | tgccattttacaaataagactt |
| | | atatttgtccttttgtttttca |
| | | gcctaccatgagaataagagaa |
| | | agaaaatgaagatcaaaagctt |
| | | attcatctgtttttctttttcg |
| | | ttggtgtaaagccaacaccctg |
| | | tctaaaaaacataaatttcttt |
| | | aatcattttgcctcttttctct |
| | | gtgcttcaattaataaaaaatg |
| | | gaaagaatctaatagagtggta |
| | | cagcactgttatttttcaaaga |
| | | tgtgttgctatcctgaaaattc |
| | | tgtaggttctgtggaagttcca |
| | | gtgttctctcttattccacttc |
| | | ggtagaggatttctagtttctt |
| | | gtgggctaattaaataaatcat |
| | | taatactcttctaagttatgga |
| | | ttataaacattcaaaataatat |
| | | tttgacattatgataattctga |
| | | ataaaagaacaaaaaccatggt |
| | | ataggtaaggaatataaaacat |
| | | ggcttttaccttagaaaaaaca |
| | | attctaaaattcatatggaatc |
| | | aaaaaagagcctgcag |
| 71 | Mutant | EDPQGDAAQKTDTSHHDQDHPT |
| | AAT- | FNKITPNLAEFAFSLYRQLAHQ |
| | linkerAlbumin | SNSTNIFFSPVSIATAFAMLSL |
| | Fusion | GTKADTHDEILEGLNFNLTEIP |
| | (S283C, K335A, | EAQIHEGFQELLRTLNQPDSQL |
| | P361C) | QLTTGNGLFLSEGLKLVDKFLE |
| | | DVKKLYHSEAFTVNFGDTEEAK |
| | | KQINDYVEKGTQGKIVDLVKEL |
| | | DRDTVFALVNYIFFKGKWERPF |
| | | EVKDTEEEDFHVDQVTTVKVPM |
| | | MKRLGMFNIQHSKKLSSWVLLM |
| | | KYLGNATAIFFLPDEGKLQHLE |
| | | NELTHDIITKFLENEDRRCASL |
| | | HLPKLSITGTYDLKSVLGQLGI |
| | | TKVFSNGADLSGVTEEAPLKLS |
| | | KAVHAAVLTIDEKGTEAAGAVF |
| | | LEAIPLSICPEVKFNKPFVFLM |
| | | IEQNTKSPLFMGKVVNPTQKGG |
| | | SGGSGGSGGSGGDAHKSEVAHR |
| | | FKDLGEENFKALVLIAFAQYLQ |
| | | QCPFEDHVKLVNEVTEFAKTCV |
| | | ADESAENCDKSLHTLFGDKLCT |
| | | VATLRETYGEMADCCAKQEPER |
| | | NECFLQHKDDNPNLPRLVRPEV |
| | | DVMCTAFHDNEETFLKKYLYEI |
| | | ARRHPYFYAPELLFFAKRYKAA |
| | | FTECCQAADKAACLLPKLDELR |
| | | DEGKASSAKQRLKCASLQKFGE |
| | | RAFKAWAVARLSQRFPKAEFAE |
| | | VSKLVTDLTKVHTECCHGDLLE |
| | | CADDRADLAKYICENQDSISSK |
| | | LKECCEKPLLEKSHCIAEVEND |
| | | EMPADLPSLAADFVESKDVCKN |
| | | YAEAKDVFLGMFLYEYARRHPD |
| | | YSVVLLLRLAKTYETTLEKCCA |
| | | AADPHECYAKVFDEFKPLVEEP |
| | | QNLIKQNCELFEQLGEYKFQNA |
| | | LLVRYTKKVPQVSTPTLVEVSR |
| | | NLGKVGSKCCKHPEAKRMPCAE |
| | | DYLSVVLNQLCVLHEKTPVSDR |
| | | VTKCCTESLVNRRPCFSALEVD |
| | | ETYVPKEFNAETFTFHADICTL |
| | | SEKERQIKKQTALVELVKHKPK |
| | | ATKEQLKAVMDDFAAFVEKCCK |
| | | ADDKETCFAEEGKKLVAASQAA |
| | | LGL |

### EXAMPLES

### Example 1: Construction of AAT-Albumin

Wildtype alpha-1-antitrypsin (alpha-1-antitrypsin, SEQ ID: 1) and four variants (C232S, SEQ ID: 64; M351V, M358V, SEQ ID: 65; M351V, M358L, SEQ ID: 66; C232S, M351V, M358L, SEQ ID: 67) are fused to the N-terminus of human serum albumin via long glycine-serine linker (GGSGGSGGSGGSGG (SEQ ID NO: 63)). The gene encoding human AAT can be PCR amplified from human liver cDNA (Zyagen). Specific point mutations within the AAT gene are created by overlapping PCR (See, for example, Higuchi R, Krummel B, Saiki R (1988) “A general method of in vitro preparation and specific mutagenesis of DNA fragments: study of protein and DNA interactions”. Nucleic Acids Res. 16 (15): 7351-67, which is incorporated by reference herein in its entirety, including any drawings.) The alpha-1-antitrypsin gene is cloned in frame with a gene encoding human serum album and a linker, into a mammalian expression vector containing a mammalian secretion signal sequence up stream of the alpha-1-antitrypsin gene insertion site.

The initial AAT variants are chosen because they are either resistant to cysteine oxidation and/or methionine oxidation. Methionine oxidation is known to decrease the AAT inhibitory activity for human neutrophil elastase. Once expressed in Chinese Hamster Ovary (CHO) cells, each AAT-human serum albumin (WT or variant, SEQ ID: 1-5) is purified using an anti-AAT resin (GE) and tested for inhibition of human neutrophil elastase (hNE). Each AAT-human serum albumin (WT or variant, SEQ ID: 1-5) is also tested for overall protein stability using a dynamic light scattering (DLS) & dynamic scanning fluorimetry (DSF) instrument (UNcle, Unchained).

Overall AAT protein stability is known to correlate with resistance to self-polymerization of AAT. With this data, AAT-human serum albumins are chosen for further mutagenesis with at least one additional mutation (See Table 1 above). These mutations are known to further increase AAT stability and prevent self-polymerization. Each AAT-human serum albumin variant is expressed in CHO, purified, and tested for hNE inhibition and protein stability.

By combining multiple mutations together, unexpected synergistic increases in stability are observed with little to no loss in hNE inhibition.

Each gene sequence was cloned into proprietary high expression mammalian vectors. Each completed construct was sequence confirmed before proceeding to DNA scale up. Each DNA expression construct was scaled up to the appropriate amount for transfection. Plasmid DNA was run on agaraose gel for quality assessment and the sequence was confirmed before proceeding to transfection.

### Example 2 Construction of AAT-Albumin Binding Domain

Preferred AAT domains are fused to an albumin binding single domain antibody instead of human serum albumin as set forth in Example 1. For example, single domain antibodies having any of respective sequences SEQ ID NOs 22-36 are fused to AAT as provided in Example 1. The AAT-albumin binding domain variant is expressed in CHO, purified, and tested for hNE inhibition and protein stability as set forth in Example 1.

Each gene sequence was cloned into proprietary high expression mammalian vectors. Each completed construct was sequence confirmed before proceeding to DNA scale up. Each DNA expression construct was scaled up to the appropriate amount for transfection. Plasmid DNA was run on agaraose gel for quality assessment and the sequence was confirmed before proceeding to transfection.

### Example 3 CHO Transient Transfection

Suspension CHO cells were seeded in a shake flask and were expanded using serum-free chemically defined medium. On the day of transfection, expanded cells were seeded into a new flask with fresh medium. Each DNA construct was transiently transfected into the CHO cells. Cells were maintained as a batch-fed culture (C1603 and C1604, Medna) until the end of the production run.

The results can be seen in Table 2 and in Table 4.

TABLE 2

| Name | V | Harvest | Titer | Final Yield | In Stock | PI |
|---|---|---|---|---|---|---|
| PP12288-J1350 | 100 mL | 50.99 mg | 509.9 mg/L | 28.14 mg | 23.10 mg | 5.6 (Calc.) |
| AAT | | | | | | |
| PP12289-J1351 | 100 mL | 43.64 mg | 436.4 mg/L | 25.74 mg | 20.70 mg | 5.6 (Calc.) |
| C232S | | | | | | |
| PP12290-J1352 | 100 mL | 43.65 mg | 436.5 mg/L | 25.58 mg | 20.42 mg | 5.6 (Calc.) |
| M351V, M358V | | | | | | |
| PP12291-J1353 | 100 mL | 46.94 mg | 469.4 mg/L | 20.98 mg | 15.90 mg | 5.6 (Calc.) |
| M351V, M358L | | | | | | |
| PP12292-J1354 | 100 mL | 47.73 mg | 477.3 mg/L | 28.89 mg | 23.52 mg | 5.6 (Calc.) |
| C232S, M351V, M358L | | | | | | |

TABLE 4

| Name | Harvest | V (L) | Titer (mg/L) | Final Yield | In-Stock | PI |
|---|---|---|---|---|---|---|
| F51L | 37.44 | 0.1 | 374.4 | 21.35 | 21.35 | 5.6 (Calc.) |
| G117F | 36.66 | 0.1 | 366.6 | 28.03 | 28.03 | 5.6 (Calc.) |
| K331F | 33.82 | 0.1 | 338.2 | 21.51 | 21.51 | 5.5 (Calc.) |
| K335A | 24.65 | 0.1 | 246.5 | 13.86 | 13.86 | 5.5 (Calc.) |
| K168C/F189C | 48.03 | 0.1 | 480.3 | 32.82 | 32.82 | 5.5 (Calc.) |
| S283C/P361C | 40.59 | 0.1 | 405.9 | 27.37 | 27.37 | 5.6 (Calc.) |
| F51L/G117F | 32.48 | 0.1 | 324.8 | 22.06 | 22.06 | 5.6 (Calc.) |
| F51L/K331F | 31.71 | 0.1 | 317.1 | 18.71 | 18.71 | 5.5 (Calc.) |
| F51L, K335A | 39.21 | 0.1 | 392.1 | 22.45 | 16.41 | 5.5 (Calc.) |
| F51L/K168C/F189C | 40.55 | 0.1 | 405.5 | 26.48 | 20.44 | 5.5 (Calc.) |
| F51L/S283C/P361C | 45.31 | 0.1 | 453.1 | 35.17 | 29.14 | 5.6 (Calc.) |
| G117F/K331F | 34.45 | 0.1 | 344.5 | 21.43 | 15.43 | 5.5 (Calc.) |
| G117F/K335A | 40.87 | 0.1 | 408.7 | 27.58 | 21.42 | 5.5 (Calc.) |
| G117F/K168C/F189C | 35.71 | 0.1 | 357.1 | 28.96 | 22.9 | 5.5 (Calc.) |
| G117F/S283C/P361C | 42.52 | 0.1 | 425.2 | 31.57 | 25.54 | 5.6 (Calc.) |
| K331F/K335A | 26.72 | 0.1 | 267.2 | 13.33 | 7.2 | 5.5 (Calc.) |
| K331F/K168C/F189C | 28 | 0.1 | 280.0 | 17.75 | 11.68 | 5.5 (Calc.) |
| K331F/S283C/P361C | 41.18 | 0.1 | 411.8 | 24.12 | 18.07 | 5.5 (Calc.) |
| K335A/K168C/F189C | 39.16 | 0.1 | 391.6 | 19.23 | 13.13 | 5.5 (Calc.) |
| K335A/S283C/P361C | 40.15 | 0.1 | 401.5 | 29.75 | 23.61 | 5.5 (Calc.) |
| K168C/F189C/S283C/P361C | 41.3 | 0.1 | 413.0 | 32.41 | 26.35 | 5.5 (Calc.) |

### Example 4 CHO Albumin Affinity Purification

Conditioned media from the transient production run was harvested and clarified by centrifugation and filtration. The supernatant was loaded over a column packed with alpha-1 antitrypsin select resin and pre-equilibrated with PBS, pH 7.4. The column was washed with PBS to remove impurities until OD280 (measured by NanoDrop, Thermo Scientific) dropped to around zero. Target protein was eluted with a high salt elution buffer (20 mM Tris, 2 M MgCl2, pH 7.4), fractions were collected and OD280 was measured and recorded. Fractions containing the target protein were pooled and dialyzed to final buffer to PBS, pH 7.4. The final product was filtered through 0.2 m membrane filter. Protein concentration and final yield were calculated from OD280 value and calculated extinction coefficient after buffer exchange.

CE-SDS analysis of target protein was performed using LabChip GXII (Perkins Elmer).

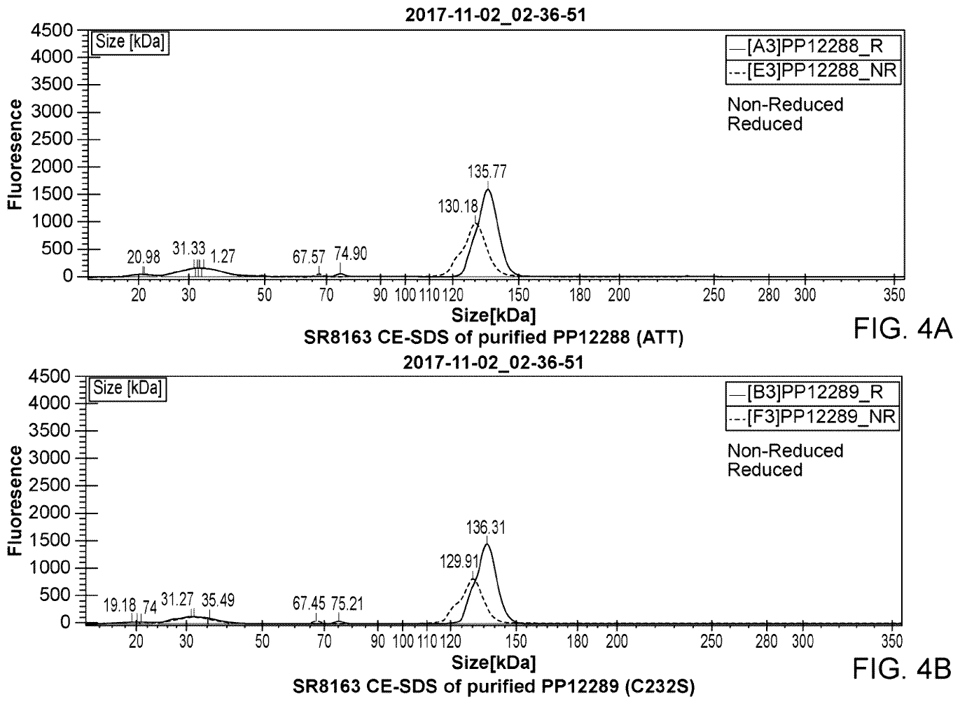

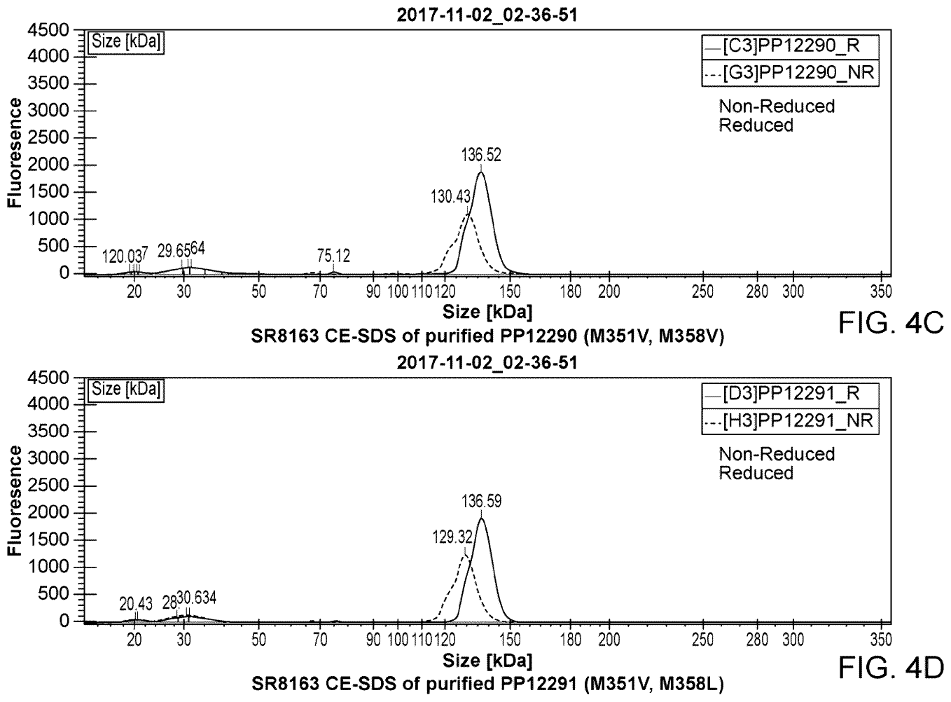

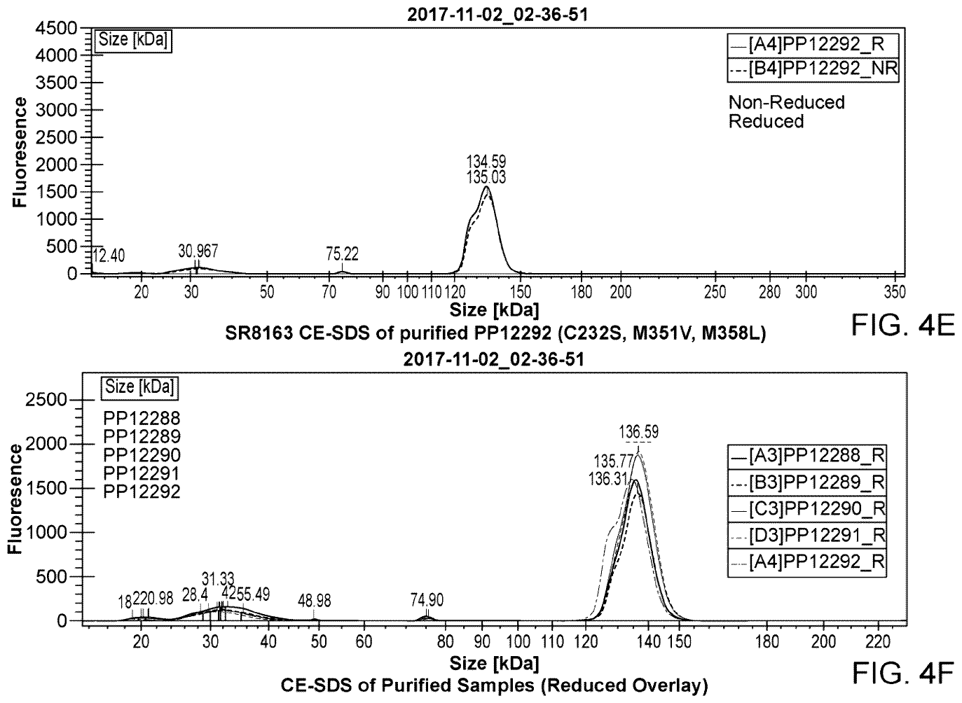

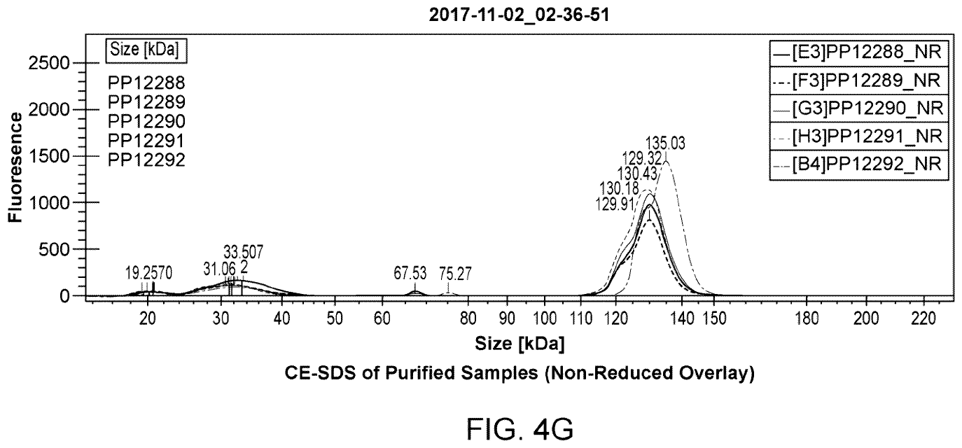

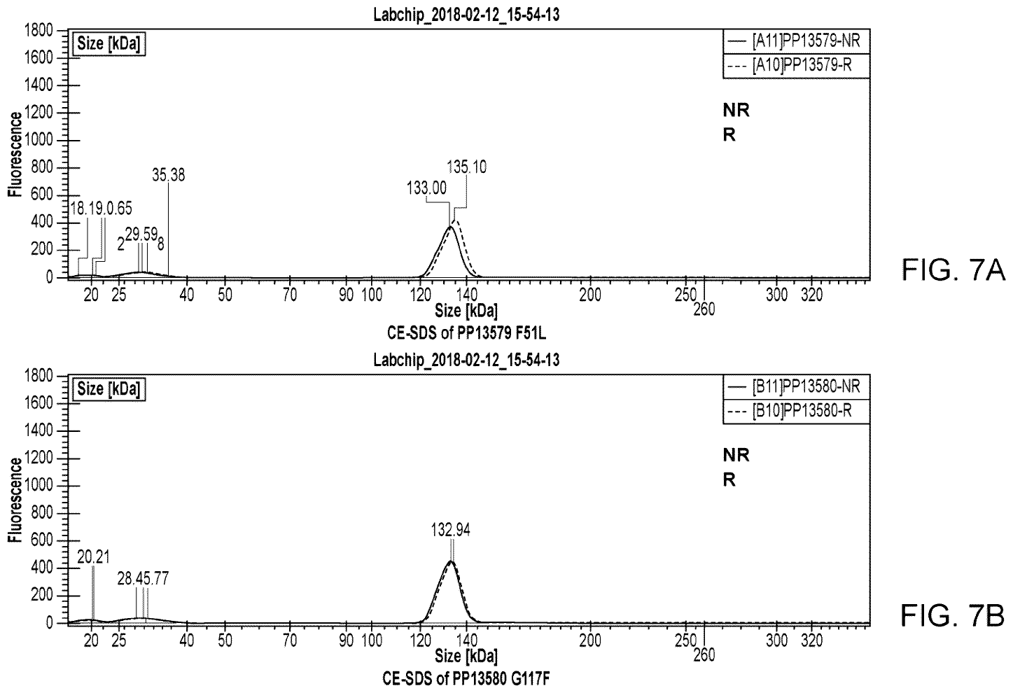

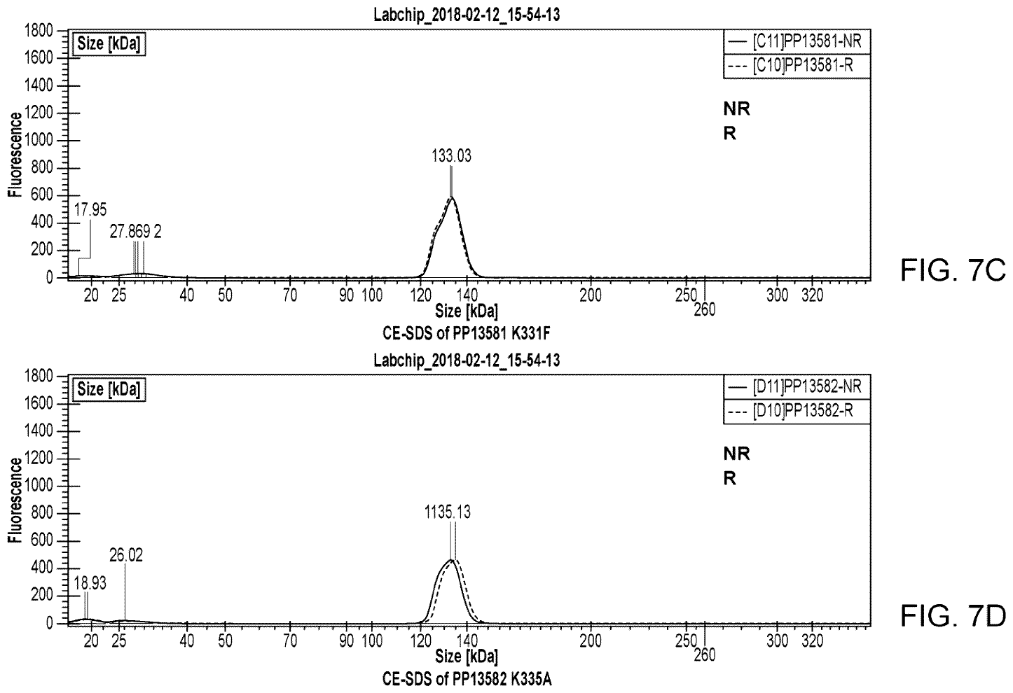

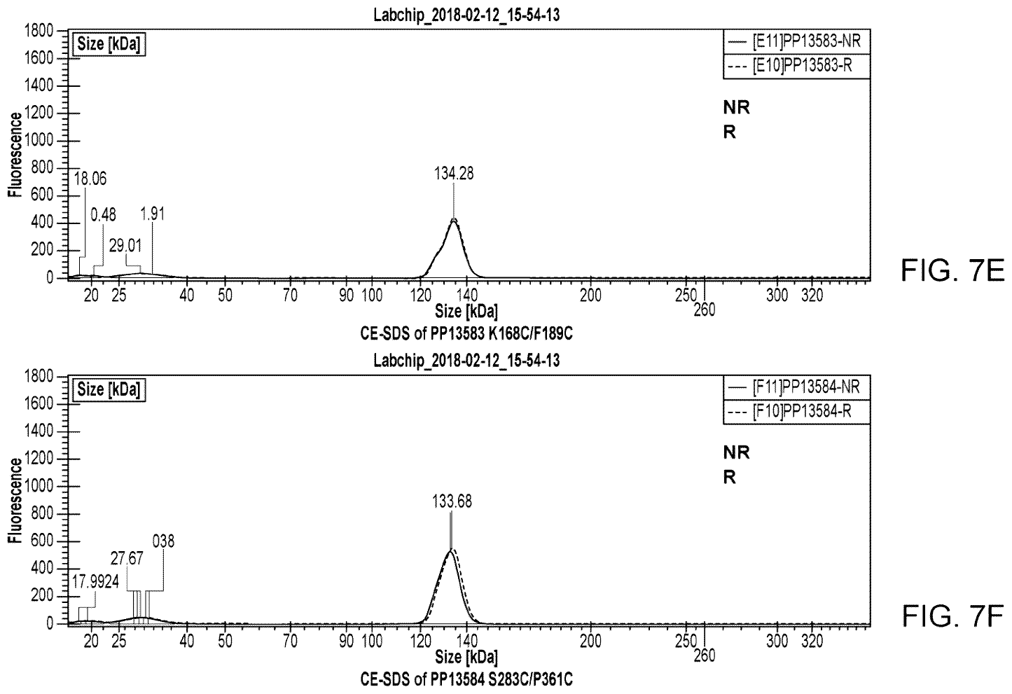

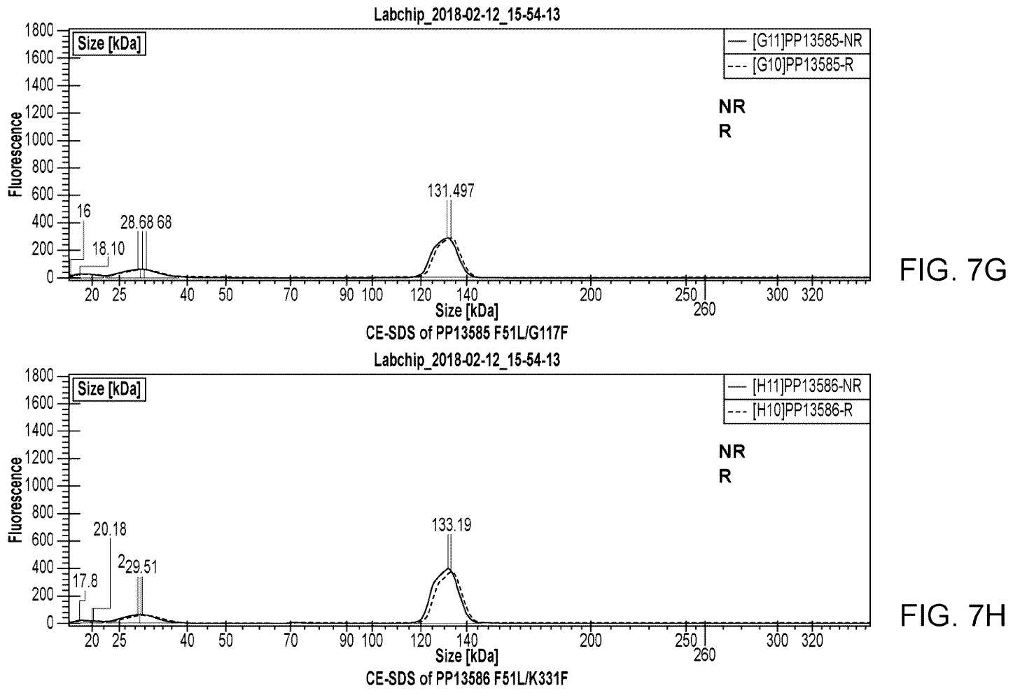

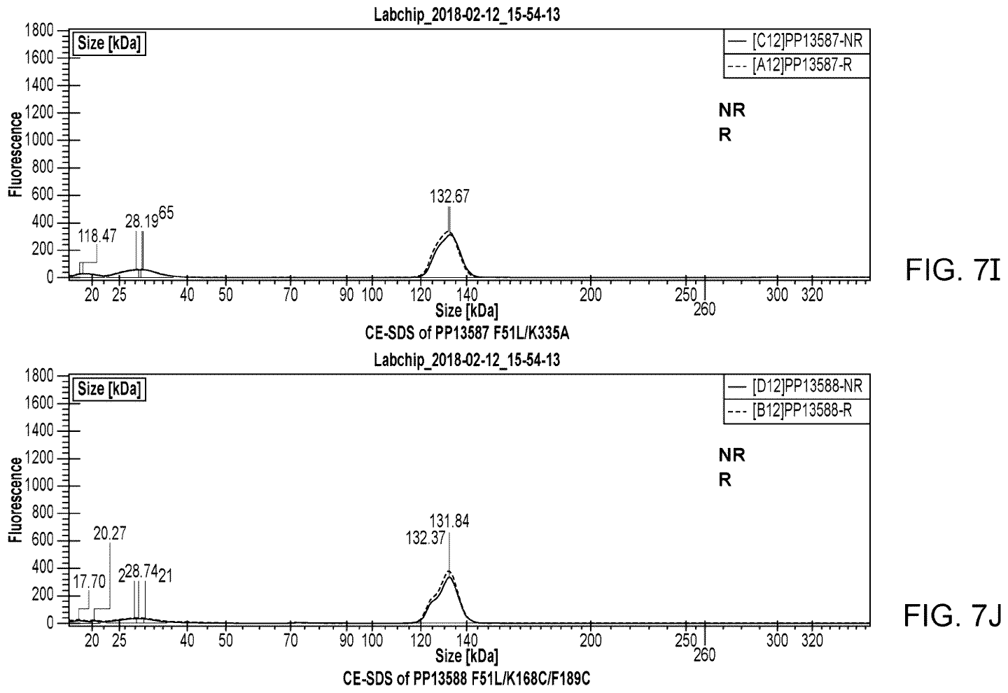

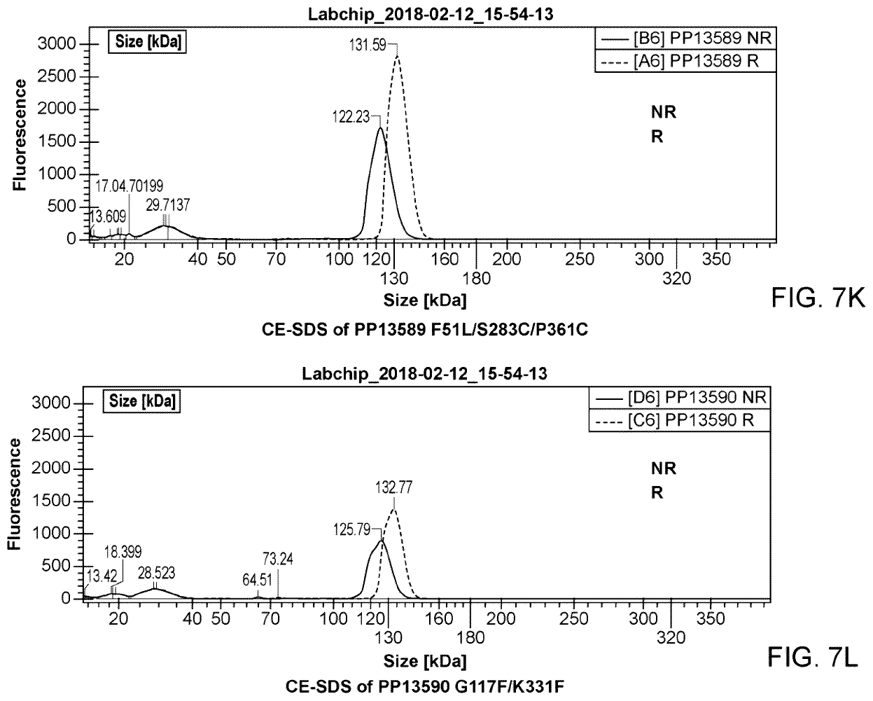

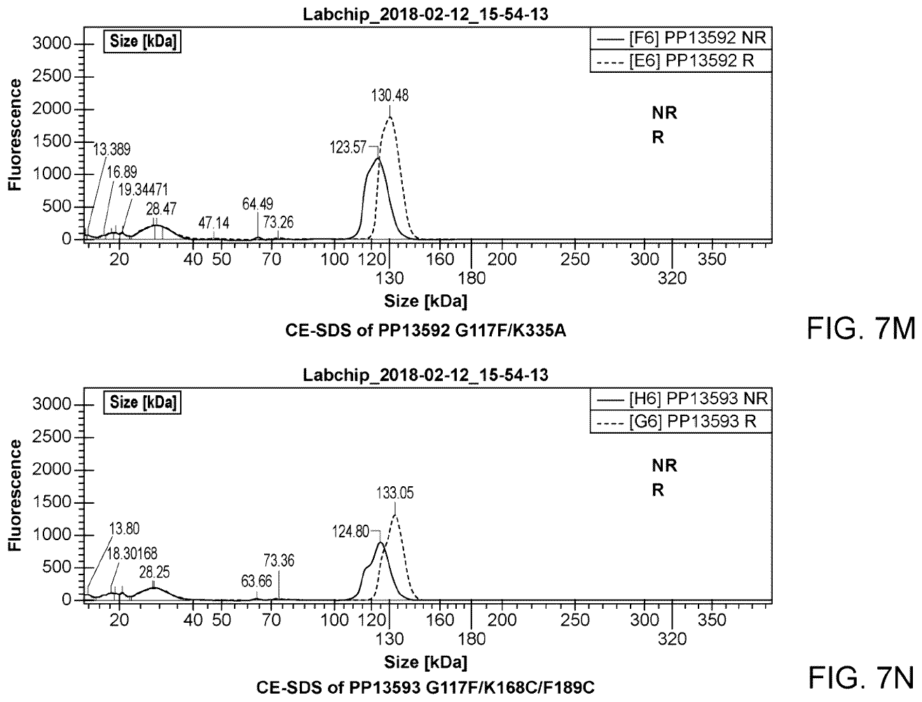

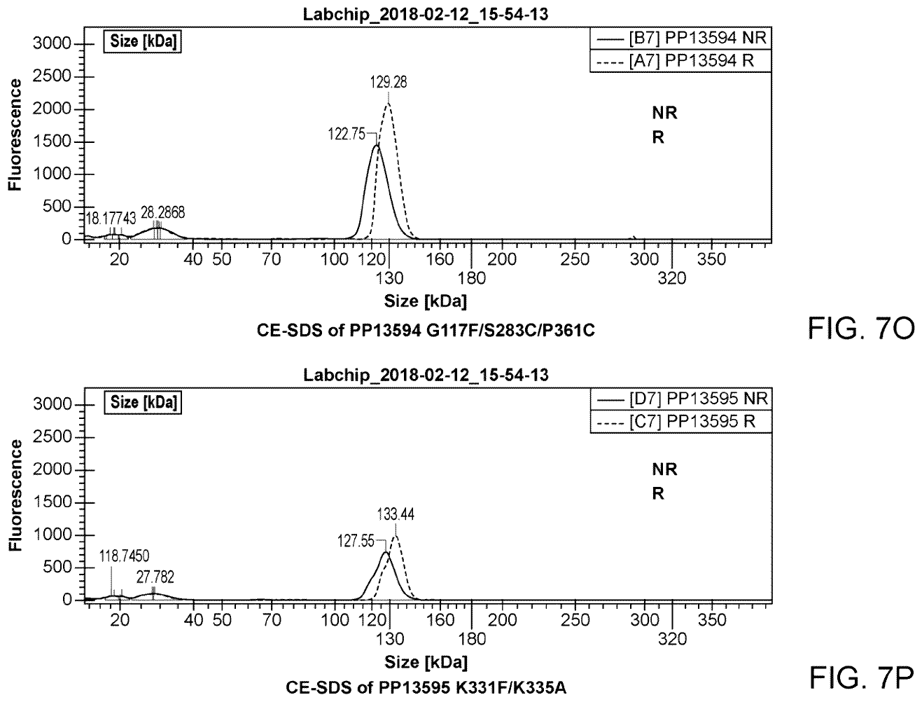

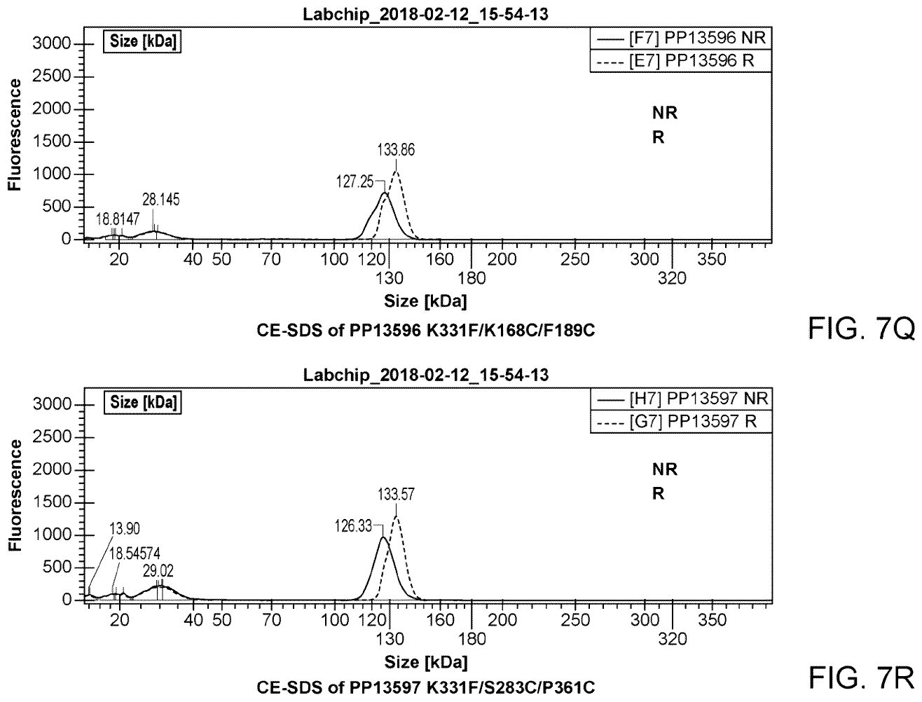

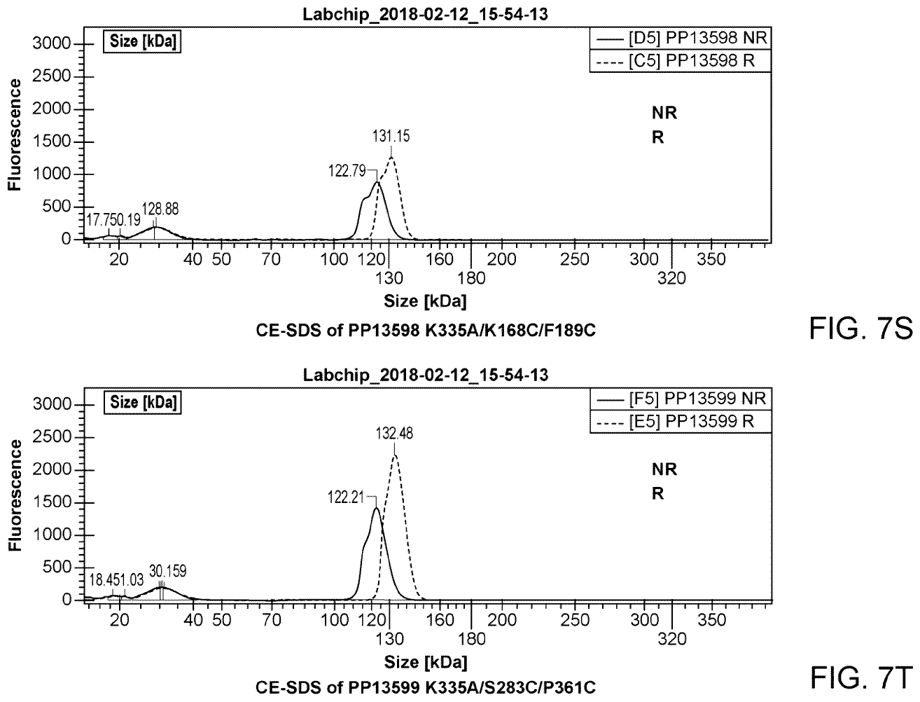

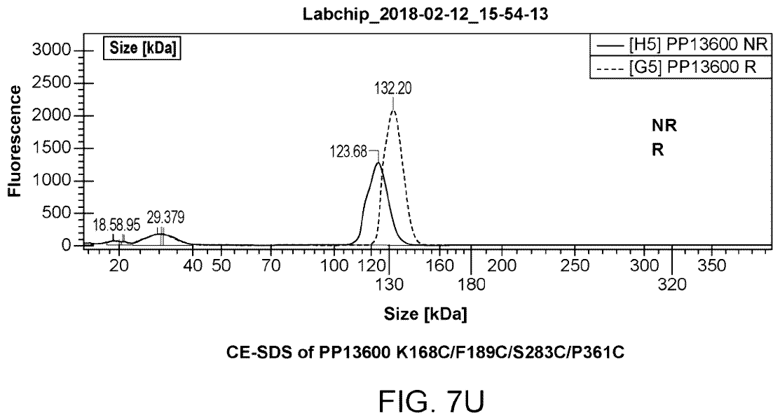

The results can be seen in FIG. 4 and in FIG. 7.

### Example 5: Stability Analysis

A sample was submitted to the UNcle system (Unchained Labs) for analysis. A temperature ramp of 1° C./min was performed with monitoring from 25° C. to 95° C. for dynamic light scattering (“DSF”) and static light scattering (“SLS”). UNcle measures SLS at 266 nm and 473 nm. DLS was measured at the beginning of thermal ramp. Tm, Tagg, and DLS measurements were calculated and analyzed by using the UNcle Analysis Software. The Tm as determined is indicated by a solid dropline in DSF graph.

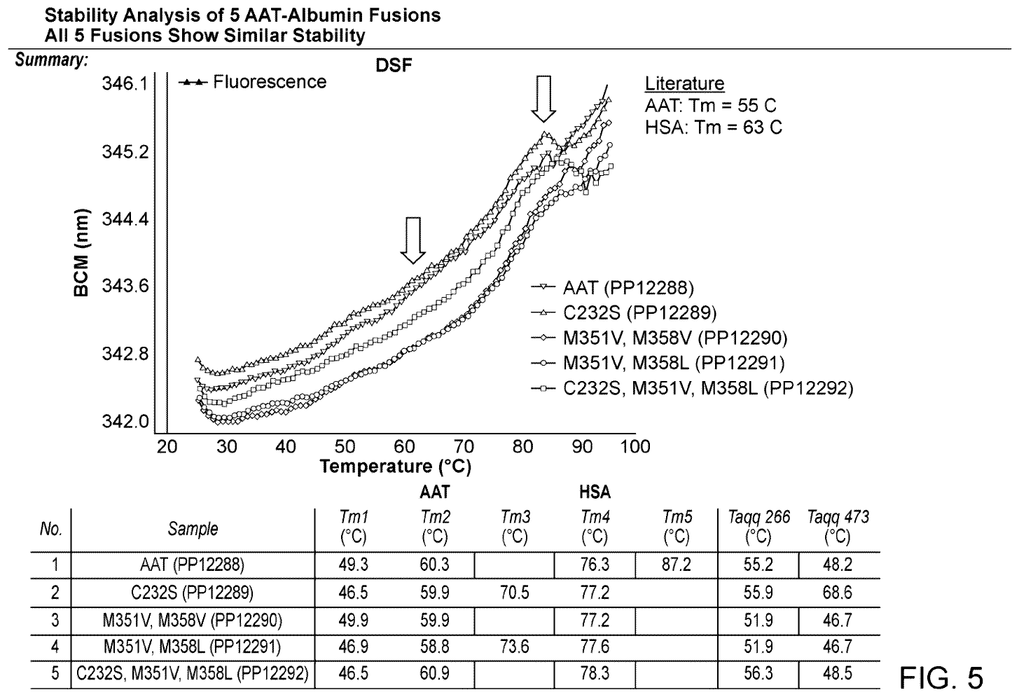

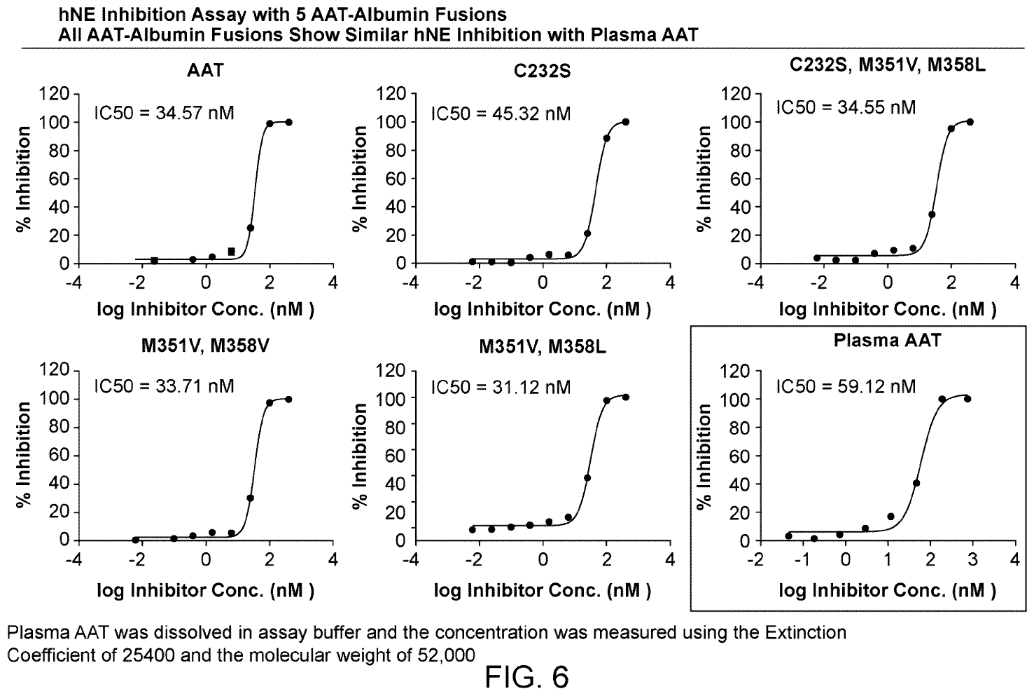

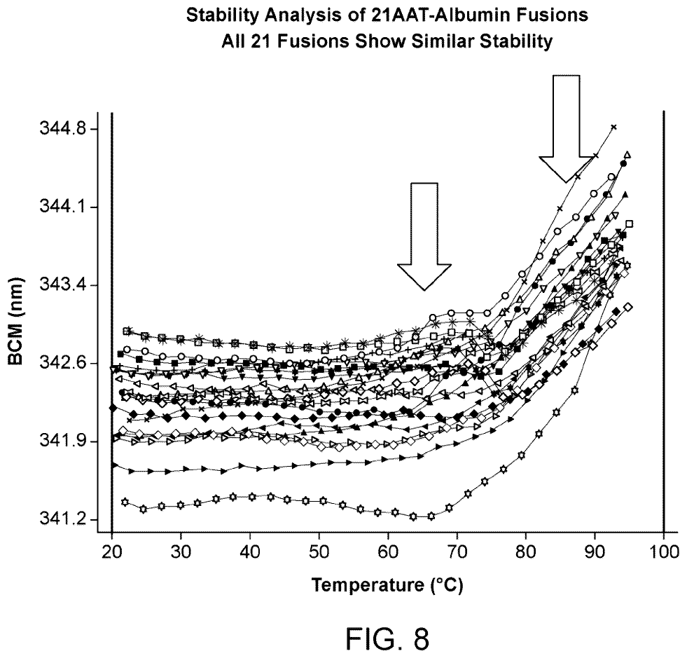

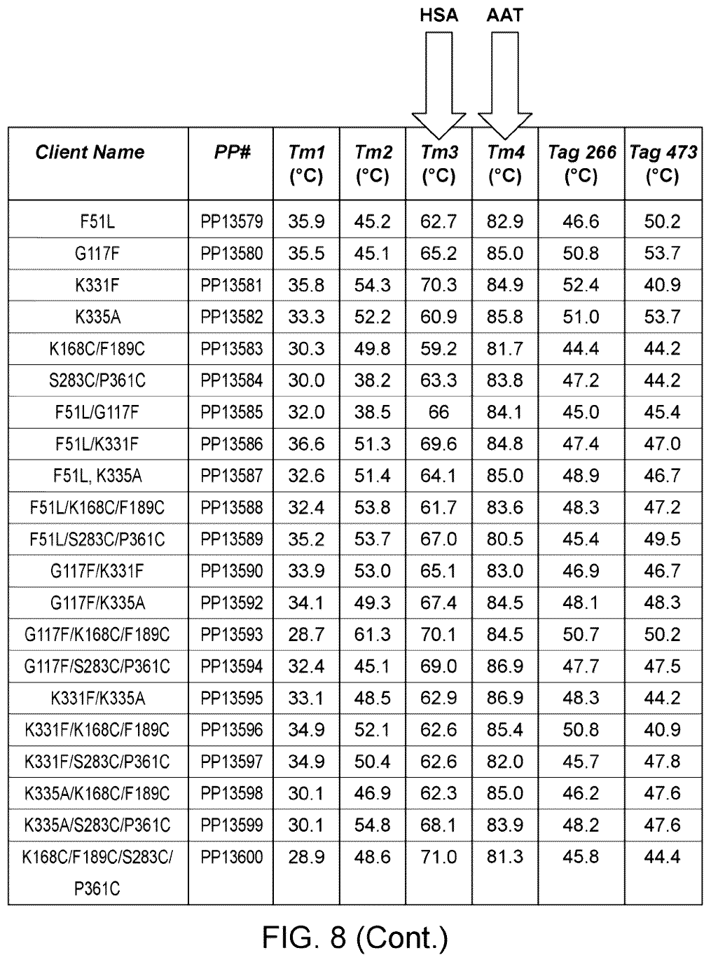

The results can be seen in FIG. 5 and in FIG. 8.

### Example 6: hNA Assay

A neutrophil elastase inhibitory assay was performed with the screening kit from Abcam (Cat #ab 118971). Results are shown in FIG. 6.

### Example 7: IC50

IC50 analysis of AAT mutants was also performed with the screening kit from Abcam (Cat #ab 118971). 400 nM-98 pM was concentration range used for IC50 analysis and data obtained from two replicates was analyzed using GraphPad Prism.

Table 3 shows IC50s for base albumin-fusion.

TABLE 3

| Name | Titer | IC50 (nM) |
|---|---|---|
| PP12288-J1350 | 509.9 mg/L | 34.57 |
| AAT | | |
| PP12289-J1351 | 436.4 mg/L | 45.32 |
| C232S | | |
| PP12290-J1352 | 436.5 mg/L | 33.71 |
| M351V, M358V | | |
| PP12291-J1353 | 469.4 mg/L | 31.12 |
| M351V, M358L | | |
| PP12292-J1354 | 477.3 mg/L | 34.55 |
| C232S, M351V, M358L | | |
| Plasma AAT | NA | 59.12 |

21 AAT mutant proteins and 1AAT serum control were also used to generate IC50 analysis curves. Table 5 shows IC50s for mutant AAT.

TABLE 5

| | | Titer | IC50 |
|---|---|---|---|
| BASE | Mutations | (mg/L) | (nM) |
| C232S, M351V, M358L | K335A | 247 | 5.96 |
| C232S, M351V, M358L | K331F | 338 | 7.05 |
| AAT Serum Control | — | — | 7.98 |
| C232S, M351V, M358L | K335A/S283C/P361C | 402 | 8.55 |
| C232S, M351V, M358L | G117F/K335A | 409 | 8.58 |
| C232S, M351V, M358L | G117F/K331F | 345 | 8.94 |
| C232S, M351V, M358L | F51L/K331F | 317 | 9.18 |
| C232S, M351V, M358L | K331F/K335A | 267 | 9.30 |
| C232S, M351V, M358L | G117F | 367 | 10.45 |
| C232S, M351V, M358L | F51L/K335A | 392 | 10.67 |
| C232S, M351V, M358L | F51L | 374 | 13.57 |
| C232S, M351V, M358L | K331F/S283C/P361C | 412 | 14.32 |
| C232S, M351V, M358L | K335A/K168C/F189C | 392 | 15.77 |
| C232S, M351V, M358L | F51L/S283C/P361C | 453 | 15.97 |
| C232S, M351V, M358L | F51L/G117F | 325 | 19.00 |
| C232S, M351V, M358L | G117F/S283C/P361C | 425 | 20.15 |
| C232S, M351V, M358L | F51L/K168C/F189C | 406 | 23.32 |
| C232S, M351V, M358L | K168C/F189C | 480 | 23.47 |
| C232S, M351V, M358L | K331F/K168C/F189C | 280 | 27.96 |
| C232S, M351V, M358L | G117F/K168C/F189C | 357 | 29.10 |
| C232S, M351V, M358L | K168C/F189C/S283C/P361C | 413 | 35.01 |
| C232S, M351V, M358L | S283C/P361C | 406 | 41.71 |

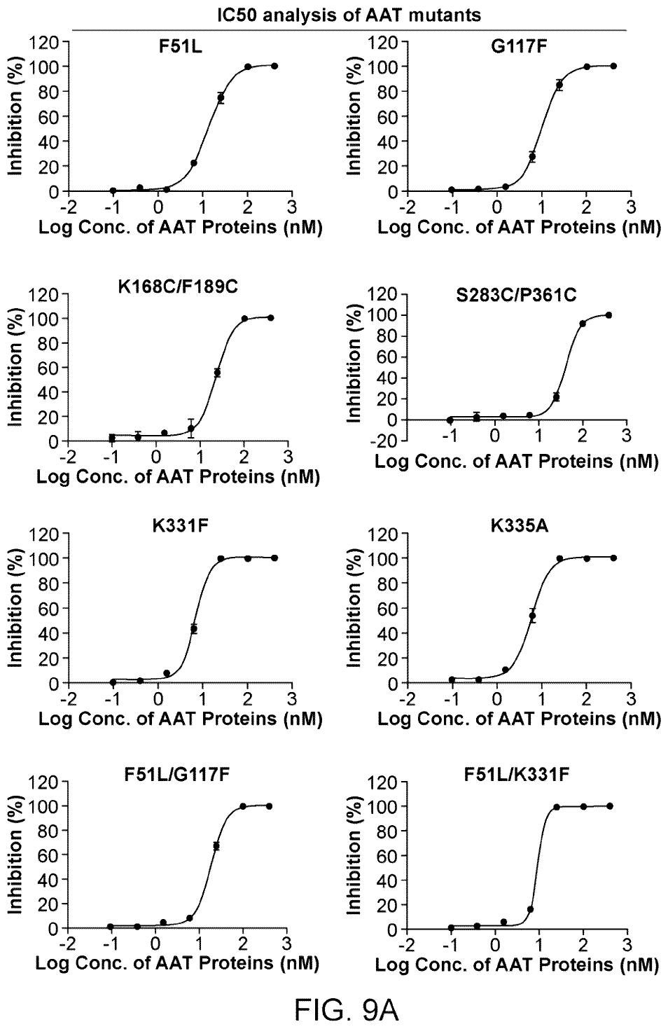

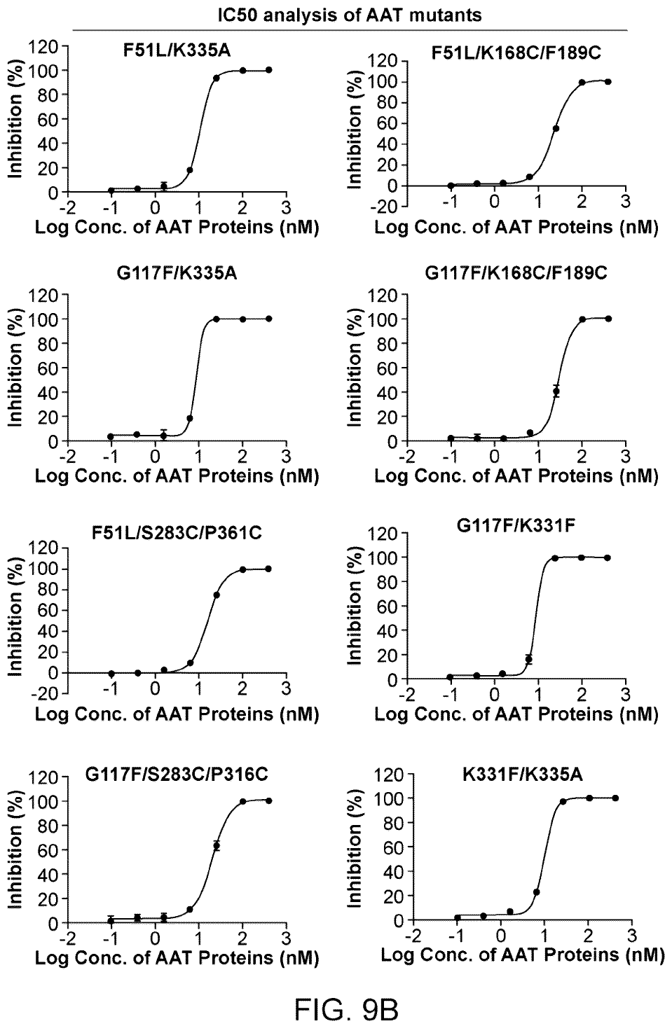

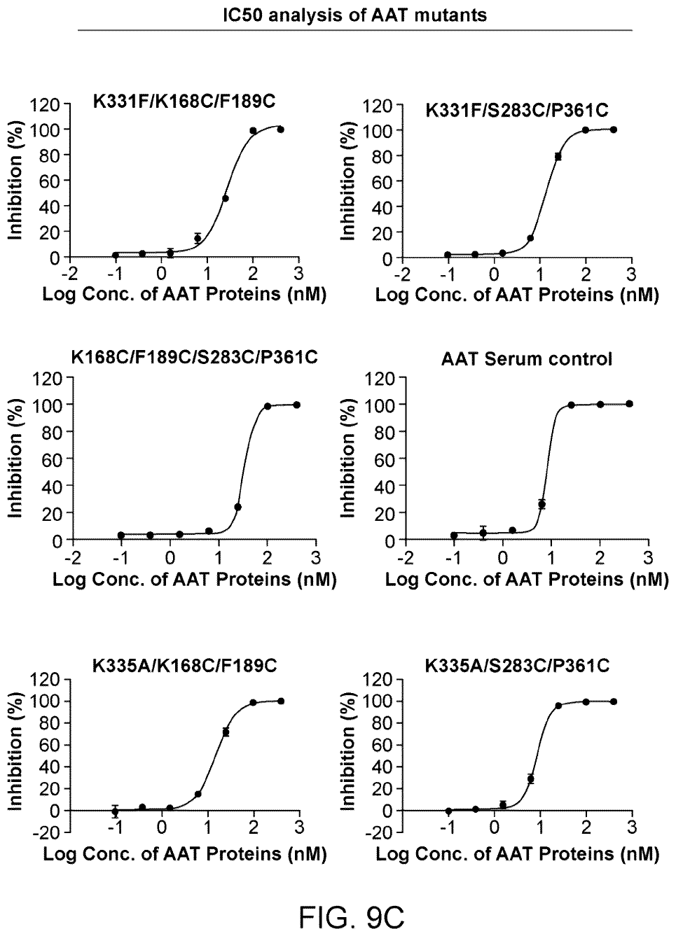

FIG. 9 shows IC50 analysis of AAT mutants, with the x-axis showing log concentration of AAT proteins (nM) and the y-axis showing percent inhibition. Curves that have a higher slope show a very cooperative inhibition profile. Mutants G11F/K335A, G117F/K331F, F51L/K331F, and K331F/K335A all have a very cooperative inhibition profile.

### Example 8: In-Silico Immunogenicity

A proprietary screen was used to compare proteins with those already in clinical use (See, Jawa et al., T-cell dependent immunogenicity of protein therapeutics: Preclinical assessment and mitigation, 149 Clinical Immunology, 534-555 (2013), which is incorporated by reference herein in its entirety, including any drawings). AAT-albumin fusions (“AAT_MLA”), AAT mutants (“AAT_Mutant”), and albumin proteins (“Albumin”) were screened for the presence of putative T-cell epitopes. The AAT Mutant has the following mutations according to SEQ ID NO: 1: C232S, M351V, M358L, K335A, S283C, and P361C. The immunogenic potential of each T-cell epitope or AAT-albumin fusions, AAT mutants, and albumin proteins were compared to othes, normalized, and ranked.

Input sequences were parsed into overlapping 9-mer frames and each frame was evaluated with respect to a panel of eight common Class II HLA alleles. The alleles are called super-types. Each super type is functionally equivalent to, or nearly equivalent to, many additional family member alleles. Taken collectively, the eight super-type alleles, along with their respective family members, cover well over 95% of the human population. Each frame-by-allele assessment is a statement about predicted HLA binding affinity.

Assessment scores range from approximately −3 to +3. Assessment scores above 1.64 are defined as “hits” and are considered potentially immunogenic and worthy of further consideration. One can often expect about 5% of all assessments to score above 1.64. These peptides have a significant chance of binding to HLA molecules with moderate to high affinity and, therefore, have a significant chance of being presented on the surface of APCs, such as dendritic cells or macrophages, where they may be interrogated by passing T cells. The greater the burden of HLA ligands (i.e. hits) contained in a given protein, the more likely that protein is to induce an immune response.

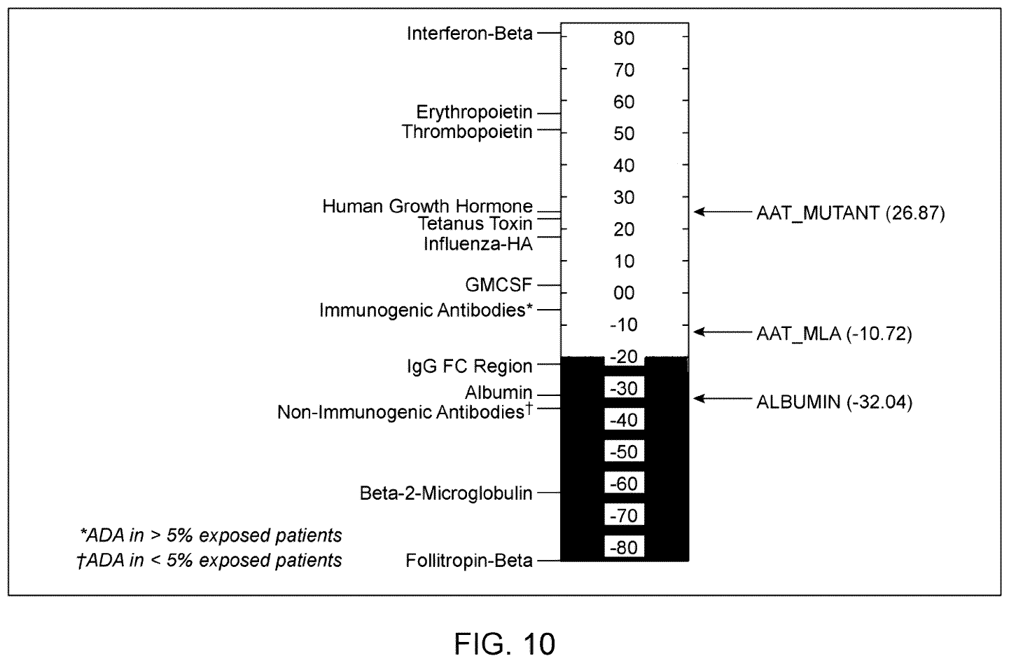

The EpiMatrix Protein Score is the difference between the number of predicted T cell epitopes one would expect to find in a protein of a given size and the number of putative epitopes predicted by the EpiMatrix System. EpiMatrix Protein Scores are “normalized” and can be plotted on a standardized scale (See, for example, FIG. 10). The EpiMatrix Protein Score of an “average” protein is zero. EpiMatrix Protein Scores above zero indicate the presence of excess MHC ligands and denote a higher potential for immunogenicity while scores below zero indicate the presence of fewer potential MHC ligands than expected and a lower potential for immunogenicity.

In analyzing the full AAT_MLA sequence, the algorithm performed a total of 7,880 frame-by-allele assessments. In analyzing the AAT_MUTANT domain, the algorithm performed a total of 3,088 frame-by-allele assessments. In analyzing the ALBUMIN domain, the algorithm performed a total of 4,616 frame-by-allele assessments. The results are shown in Table 6. Mutant AAT shows a marginally increased immunogenicity score compared to wild type AAT. The fusion of high-scoring AAT_MUTANT domain to the low-scoring glycine-serine linker and low-scoring ALBUMIN domain lowers the overall epitope density of the AAT_MLA fusion. AAT, when fused to albumin (AAT_MLA), has a negative score, indicating reduced immunogenic potential compared to wild type or mutant AAT alone.

TABLE 6

| | | | EpiMatrix | EpiMatrix |
|---|---|---|---|---|
| Protein Label | Length | Assessments | Hits | Score |
| AAT MLA | 993 | 7,880 | 384 | −10.72 |
| AAT MUTANT | 394 | 3,088 | 206 | 26.87 |
| AAT WT | 394 | 3,088 | 202 | 21.01 |
| ALBUMIN | 585 | 4,616 | 178 | −32.04 |

384 peptide-to-HLA hits were identified within the full fusion sequence AAT_MLA, resulting in an overall score of −10.72. 206 hits were identified within the full fusion sequence AAT_MUTANT, resulting in an overall score of 26.87. 202 hits were identified within the full fusion sequence AAT_WT, resulting in an overall score of 21.01. 178 hits were identified within the full fusion sequence AAT_MLA, resulting in an overall score of −32.04. The results are shown in FIG. 10.

### Example 9: Stability Analysis of AAT Variant (K335A, S283C/P361C) on Base (C232S, M351V, M351L)

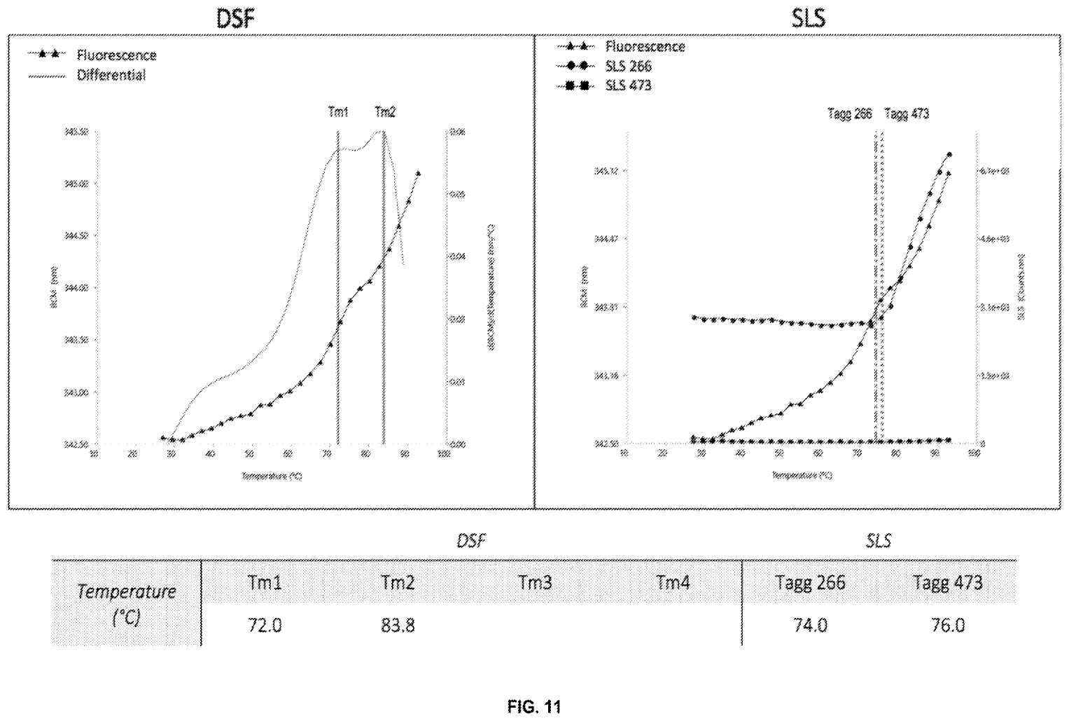

Sample was submitted to UNcle (Unchained Labs) for analysis. A temperature range of 1° C./mi was performed while being monitored from 25° C. to 95° C. for DSF and SLS. UNcle measures SLS at 266 nm and 473 nm. DLS was measured at the beginning of the thermal ramp. Tm, Tagg, and DLS measurements were calculated and analyzed by using the UNcle analysis software. Tm was determined as indicated by a solid dropline in FIG. 11. As can be seen, the variant AAT albumin fusion is very stable.

### Example 10: Cyno PK Potential Toxicity of Recombinant Alpha-1-Antitrypsin (AAT)

K335A/S283C/P361C fused to human serum albumin (AAT Albumin Fusion) when given once by intravenous bolus injection for to cynomolgus monkeys was determined. The cynomolgus monkey was chosen as the animal model for this study as it is an accepted nonrodent species for preclinical toxicity testing by regulatory agencies and most accurately reflects the pharmacokinetics and pharmacodynamics of the test agent expected in human subjects. Pharmacokinetic characteristics of the AAT Albumin Fusion, compared to human plasma-derived AAT (Prolastin C) was also determined.

Table 7 provides the test articles and control article.

TABLE 7

| | Test Article-1 | Test Article-2 | Control Article |
|---|---|---|---|
| Identification | Recombinant AAT | Human Plasma- | 137 mM NaCl |
| | K335A/S283C/P361C | Derived AAT | 2.7 mM KCl |
| | fused to human | (Prolastin C) | |
| Physical | Liquid | Powder | Liquid |
| Description | | | |
| Concen- | 7.58 mg/mL | 1129 mg, dilute | Not applicable |
| tration | | | |

The control article was used for dilution of the test articles. Test article dosing formulations of 2 mg/mL were prepared to meet dose level requirements by diluting the test article stock (7.8 mg/mL), less than or equal to 4-fold serial dilution with the control article. Dose levels were selected based on the previous published studies (Journal of Chronic Obstructive Pulmonary Disease (2013) 10(6):687-95; Lancet (2015) 386(9991):360-8). Therapy with 60 mg/kg weekly as an intravenous (IV) infusion of human plasma-derived alpha-1 antitrypsin (AAT), is approved in individuals with alpha-1 antitrypsin deficiency. The safety and pharmacokinetic profile of weekly infusions of a 120 mg/kg dose AAT was considered to be safe and well tolerated in adults with AATD.

Animals were randomized and assigned to groups using a computer-based procedure prior to transfer to study. The experimental design used is provided in Table 8.

TABLE 8

| Group | Test Material | Dose Level | Dose | Dose | No. of |
|---|---|---|---|---|---|
| 1 | Recombinant AAT | 10 | 5 | 2 | 2 |
| | K335A/S283C/P361C | | | | |
| 2 | Human Plasma-Derived | 10 | 0.2 | 50 | 2 |

Test articles were administered to animals via intravenous (slow bolus) injection once on Day 1. The dose volume for each animal was based on the most recent body weight measurement. The animals were temporarily restrained for dose administration and were not be sedated.

Blood was collected by venipuncture. Samples were collected according to Table 9.

TABLE 9

| Group Nos. | Time Point | Hematology | Clinical Chemistry |
|---|---|---|---|
| All animals | Pretreatment | X | X |
| 1 and 2 | Day 29 | X | X |

Hematology was performed with a target volume of 1 mL and an anticoagulant of Potassium (K3) EDTA. A blood smear was prepared from each hematology sample. Smears were examined to assess an animal's health following. No animals were deemed unhealthy.

Clinical chemistry was performed with a target volume of 2 mL and without an anticoagulant.

Samples were collected according to Table 10.

TABLE 10
Sample Collection Time Points

| Group No(s). | Study Day/Week | Time Points (Relative to Dosing) |
|---|---|---|
| All Animals | Pretreatment | — |
| 1 to 2 | Day 1 | Day 1: 1, 6, and 12 hr post |
| | Day 2 | Day 1: 24 hr post |
| | Day 3 | Day 1: 48 hr post |
| | Day 4 | Day 1: 72 hr post |
| | Day 5 | Day 1: 96 hr post |
| | Day 8 | Day 1: 168 hr post |
| | Day 11 | Day 1: 240 hr post |
| | Day 15 | Day 1: 336 hr post |

Plasma from the samples was analyzed for concentration of test articles Bronchoalveolar lavage samples were collected from anesthetized animals and will be analyzed (data not shown).

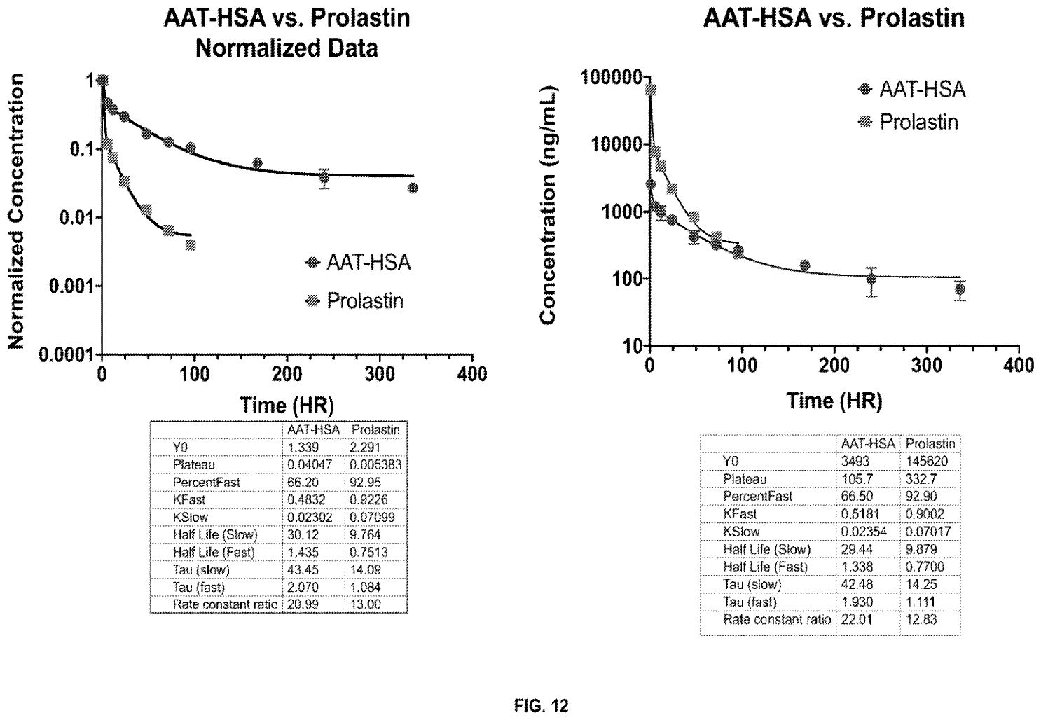

Plasma results are shown in FIG. 12 and were fitted to a 2 phase exponential decay model in Graphpad Prism. As can be seen from FIG. 12, the variant has a significantly longer terminal half life. The AAT-HSA variant (Test Article 1) half-life (beta phase, slow) was over 30 hours compared to less 10 hours for Prolastin (plasma AAT).

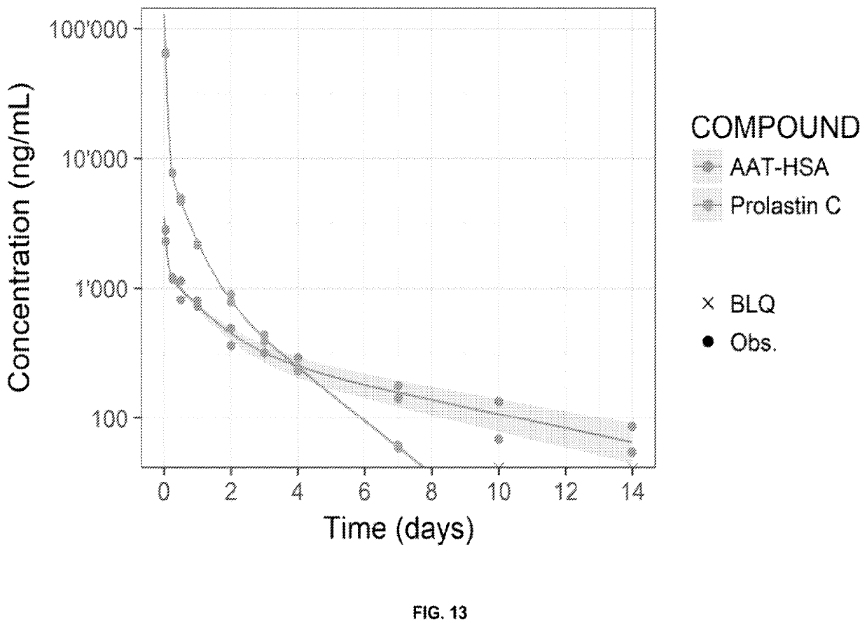

The plasma results were further analyzed by a three compartment model (Tables 11-13) and are also shown in FIG. 13. FIG. 13 shows median (lines), 5th and 95th percentiles (areas) from 1000 simulated PK profiles together with the individual observations (symbols). Observations below limit of quantification (BLQ, crosses) were not included in the analysis.

As can be seen from above, monkey PK after 10 mg/kg was three compartmental. Cₘₐₓ of test article 2 was ˜40-fold higher than the Cₘₐₓ of test article 1 for 10 mg/kg of compound. The central volume of distribution was 0.213 L for test article 2 and 8 L for test article 1. Total volumes of distribution were 1.45 L for test article 2 and 42 L for test article 1. Volume of distribution for Aralast (plasma derived AAT) in human was 5.6 L [https://www.drugbank.ca/drugs/DB00058]. Allometric scaling to NHP would predict a volume of distribution of 0.240 L, which is equivalent to test article 2 from this study.

Terminal HSA half-life in monkey is approximately 5 days. Test article 1 β and γ half-lives were 0.75 days and 5.8 days, respectively. Test article 2 β and γ half-lives were 0.36 days and 1.49 days, respectively. The test article 2 γ half-life is approximately 3.9 times longer than the γ half-life of test article 1. The γ terminal half-life of HSA (from the literature) and test article 1 are equivalent in monkey.

Table 11 shows PK parameters for test article 2 and test article 1.

TABLE 11

| Description | Parameter | Test article 2 | Test article 1 |
|---|---|---|---|
| Clearance | C1 (mL/h) | 87.3 | 295 |
| Central volume of distribution | V1 (mL) | 213 | 8020 |
| 1^{st} peripheral volume of | V2 (mL) | 749 | 23600 |
| distribution | | | |
| 2^{nd} peripheral volume of | V3 (mL) | 485 | 10800 |
| distribution | | | |
| 1^{st} inter-compartmental | Q12 (mL/h) | 18.2 | 320 |
| exchange coefficient | | | |
| 2^{nd} inter-compartmental | Q13 (mL/h) | 65.3 | 3′090 |
| exchange coefficient | | | |
| α half life | T_{1/2,α} (h) | 0.80 | 0.97 |
| β half life | T_{1/2,β} (h) | 8.64 | 18.1 |
| γ half life | T_{1/2,γ} (h) | 35.7 | 134 |

Table 12 shows population PK parameters for test article 2:

TABLE 12

| | | | |
|---|---|---|---|
| Parameter | Parameter | Estimation (% CV) | RSE |

Fixed effects

| | | | | |
|---|---|---|---|---|
| Clearance | C1 (mL/h) | 87.3 | (1) | 2.36 |
| Central volume of distribution | V1 (mL) | 213 | (0) | 10.6 |
| Inter-compartmental clearance | Q2 (mL/h) | 18.2 | (0) | 9.1 |
| Peripheral volume of distribution | V2 (mL) | 749 | (0) | 5.71 |
| 2nd inter-compartmental | Q3 (mL/h) | 65.3 | (0) | 9.41 |
| clearance | | | | |
| 2nd peripheral volume of | V3 (mL) | 485 | (0) | 7.21 |
| distribution | | | | |

Standard deviations

| | | | |
|---|---|---|---|
| Clearance | ω_{C1} | 0.0128 | 65.1 |

Observational error

| | | | |
|---|---|---|---|
| Proportional error | b | 0.0399 | 19.6 |

Table 13 shows population PK parameters for test article 1:

TABLE 13

| | | | |
|---|---|---|---|
| Parameter | Parameter | Estimation (% CV) | RSE |

Fixed effects

| | | | | |
|---|---|---|---|---|
| Clearance | C1 (mL/h) | 295 | (12) | 9.05 |
| Central volume of distribution | V1 (mL) | 8020 | (0) | 24.5 |
| Inter-compartmental clearance | Q2 (mL/h) | 320 | (0) | 20.6 |
| Peripheral volume of distribution | V2 (mL) | 23600 | (0) | 9.22 |
| 2nd inter-compartmental | Q3 (mL/h) | 3090 | (0) | 48.1 |
| clearance | | | | |
| 2nd peripheral volume of | V3 (mL) | 10800 | (0) | 20.3 |
| distribution | | | | |

Standard deviations

| | | | |
|---|---|---|---|
| Clearance | ω_{C1} | 0.122 | 53 |

Observational error

| | | | |
|---|---|---|---|
| Proportional error | b | 0.0987 | 16.8 |

1. A recombinant protein comprising:
(a) an alpha-1-antitrypsin serpin domain; and
(b) a human serum albumin binding domain.

2. The recombinant protein according to claim 1, further comprising a linker consisting of SEQ ID NO: 63.

3. The recombinant protein of any one of the preceding claims, wherein the C-terminus of the alpha-1-antitrypsin serpin domain is fused to the N-terminus of the human serum albumin binding domain.

4. The recombinant protein of any one of the preceding claims, wherein the human serum albumin binding domain is at least a portion of a human serum albumin having either SEQ ID NO: 3 or SEQ ID NO: 5.

5. The recombinant protein of any one of the preceding claims, wherein half-life of the alpha-1-antitrypsin serpin domain compared to wild type alpha-1-antitrypsin is increased.

6. The recombinant protein of any one of the preceding claims, wherein a number of N-glycans or polysialyation of the recombinant protein in the alpha-1-antitrypsin serpin domain is greater than a wild type alpha-1-antitrypsin serpin domain.

7. The recombinant protein according to any one of the preceding claims, wherein the alpha-1-antitrypsin serpin domain has one or more mutations as compared to a wild type alpha-1-antitrypsin serpin domain.

8. The recombinant protein of claim 7, wherein the one or more mutations causes resistance to methionine oxidation as compared to a wild type alpha-1-antitrypsin serpin domain.

9. The recombinant protein of claim 7 or claim 8, wherein the alpha-1-antitrypsin serpin domain retains activity compared to a wild type alpha-1-antitrypsin serpin domain.

10. The recombinant protein of claim 7 or claim 8, wherein the one or more mutations comprise M351V and/or M358V with residues being numbered according to SEQ ID NO: 1.

11. The recombinant protein of any one of claims 7-10, wherein the one or more mutations comprise K168C and F189C, with residues being numbered according to SEQ ID NO: 1, and the one or more mutations confer an increased resistance to loop-sheet polymerization as compared to wild type alpha-1-antitrypsin.

12. The recombinant protein of claim any one of claims 7-11, wherein the one or more mutations are one or more point mutations selected from F5IL, G117F, K331F, or K335A with residues being numbered according to SEQ ID NO: 1.

13. The recombinant protein of any one of the preceding claims, wherein the alpha-1-antitrypsin retains activity against human neutrophil elastase and/or PR3 compared to wild type alpha-1-antitrypsin.

14. The recombinant protein according to any one of the preceding claims, wherein the human serum albumin binding domain comprises a polypeptide having a sequence set forth in any one of SEQ ID NOs: 22-36.

15. An isolated nucleic acid encoding the recombinant protein of any of the preceding claims.

16. A vector comprising the isolated nucleic acid of claim 15.

17. A host cell comprising the vector of claim 16.

18. A method of making a recombinant protein comprising culturing the host cell of claim 17 and collecting the recombinant protein.

19. The method of claim 17 or claim 18, wherein the host cell is a mammalian host cell.

20. The method of 19, wherein the mammalian host is a CHO cell.

21. The method of any one of claims 18-20, wherein the recombinant protein is collected using an alpha-1-antitrypsin-Fc capture select media at neutral pH.

22. A method of treating or prophylactically treating an alpha-1-antitrypsin deficiency in a patient in need thereof comprising administering a recombinant protein of any one of claims 1-14 to the patient.

23. The method of claim 22, wherein administration of the recombinant protein is administered about once a week or at longer intervals than about once a week, about once every 10 days or at longer intervals than about once every ten days, about once every 15 days or at longer intervals than about once every 15 days, about once every 20 days or at longer intervals than about once every 20 days, about once every 25 days or at longer intervals than about once every 25 days, about once every month or at longer intervals than about once a month, or about once every two months or at longer intervals than about once every two months.

24. A recombinant protein comprising:
(a) an alpha-1-antitrypsin serpin domain; and
(b) a human serum albumin domain.

25. The recombinant protein of claim 24, wherein the human serum albumin domain is a wild-type human serum albumin.

26. The recombinant protein of any one of claims 24-25 comprising a linker having a sequence set forth in SEQ ID NO: 63.

27. The recombinant protein of any one of claims 24-26, wherein the C-terminus of the alpha-1-antitrypsin serpin domain is fused to the N-terminus of the human serum albumin domain.

28. The recombinant protein of any one of claims 24-27, wherein the human serum albumin domain increases plasma half-life of the alpha-1-antitrypsin serpin domain as compared to wild type alpha-1-antitrypsin.

29. The recombinant protein of any one of claims 24-28, wherein a number of N glycans or polysialyation of the recombinant protein in the alpha-1-antitrypsin serpin domain is greater than a wild type alpha-1-antitrypsin serpin domain.

30. The recombinant protein of any one claims 24-29, wherein the alpha-1-antitrypsin serpin domain has one or more mutations compared to a wild type alpha-1 antitrypsin.

31. The recombinant protein of claim 30, wherein the one or more mutations causes resistance to methionine oxidation compared to wild type alpha-1-antitrypsin.

32. The recombinant protein of claim 30 or claim 31, wherein the one or more mutations comprises C232S, M351V, M358L, and/or M358V with residues being numbered according to SEQ ID NO: 1.

33. The recombinant protein of any one of claims 30-32, wherein the one or more mutations comprises K168C and F189C, with residues being numbered according to SEQ ID NO: 1, and the one or more mutations confers a substantially increased alpha-1-antitrypsin resistance to loop-sheet polymerization, as compared to wild type alpha-1-antitrypsin.

34. The recombinant protein of any one of claims 30-33, wherein the one or more mutations are one or more point mutations selected from F51L, G117F, S283C, K331F, K335A, or P361C with residues being numbered according to SEQ ID NO: 1.

35. The recombinant protein of any of claims 30-34, wherein the one or more mutations comprises mutations selected from C232S according to SEQ ID NO: 1, M351V and M358V according to SEQ ID NO: 1, M351V and M358L according to SEQ ID NO: 1 and C232S, M351V, and M358V according to SEQ ID NO: 1.

36. The recombinant protein of any of claims 30-35, wherein the alpha-1 antitrypsin serpin domain has one or more mutations comprising F51L, C232S, M351V and M358L according to SEQ ID NO: 1, one or more mutations comprising G117F, C232S, M351V and M358L according to SEQ ID NO: 1, one or more mutations comprising C232S, K331F, M351V and M358L according to SEQ ID NO: 1, one or more mutations comprising C232S, K335A, M351V and M358L according to SEQ ID NO: 1, one or more mutations comprising K168C, F189C, C232S, M351V, and M358L according to SEQ ID NO: 1, one or more mutations comprising C232S, S283C, M351V, M358L, and P361C according to SEQ ID NO: 1, one or more mutations comprising F51L, G117F, C232S, M351V, and M358L according to SEQ ID NO: 1, one or more mutations comprising F51L, C232S, K331F, M351V, and M358L according to SEQ ID NO: 1, one or more mutations comprising F51L, C232S, K335A, M351V, and M358L according to SEQ ID NO: 1, one or more mutations comprising K51L, K168C, F189C, C232S, M351V, and M358L according to SEQ ID NO: 1, one or more mutations comprising F51L, C232S, S283C, M351V, M358L, and P361C according to SEQ ID NO: 1, one or more mutations comprising G117F, C232S, K331F, M351V, and M358L according to SEQ ID NO: 1, one or more mutations comprising G117F, K335A, C232S, M351V, and M358L according to SEQ ID NO: 1, one or more mutations comprising G117F, K168C, F189C, C232S, M351V, and M358L according to SEQ ID NO: 1, one or more mutations comprising G117F, C232S, S283C, M351V, M358L, and P361C according to SEQ ID NO: 1, one or more mutations comprising C232S, K331F, K335A, M351V, and M358L according to SEQ ID NO: 1, one or more mutations comprising K168C, F189C, C232S, K331F, M351V, and M358L according to SEQ ID NO: 1, one or more mutations comprising C232S, S283C, K331F, M351V, M358L, and P361C according to SEQ ID NO: 1, one or more mutations comprising K168C, F189C, C232S, K335A, M351V, and M358L according to SEQ ID NO: 1, one or more mutations comprising C232S, S283C, K335A, M351V, M358L, and P361C according to SEQ ID NO: 1, or one or more mutations comprising K168C, F189C, C232S, S283C, M351V, M358L, and P361C according to SEQ ID NO: 1.

37. The recombinant protein of any one of claims 24-36, wherein the alpha-1 antitrypsin retains activity as compared to the wild type alpha-1-antitrypsin.

38. The recombinant protein of any one of claims 24-37, wherein the alpha-1 antitrypsin retains activity against human neutrophil elastase and/or PR3 compared to wild type alpha-1-antitrypsin.

39. An isolated nucleic acid encoding the recombinant protein of any of claims 24-38.

40. A vector comprising the isolated nucleic acid of claim 39.

41. A host cell comprising the vector of claim 40.

42. A method of making a recombinant protein comprising culturing the host cell of claim 41 and collecting the recombinant fusion protein.

43. The method of claim 42, wherein the host cell is a eukaryotic cell.

44. The method of claim 43, wherein the eukayotic cell is a yeast cell.

45. The method of claim 44, wherein the host cell is a mammalian host cell.

46. The method of 45, wherein the mammalian host is a CHO cell.

47. The method of any one of claims 40-46, wherein the recombinant protein is collected using an alpha-1-antitrypsin-Fc select media at neutral pH.

48. A method of treating or prophylactically treating an alpha-1-antitrypsin deficiency in a patient in need thereof comprising administering the recombinant protein of any one of claims 1-14 or claims 24-38 to the patient.

49. The method of claim 48, wherein administration of the recombinant protein is administered about once a week or at longer intervals than about once a week, about once every 10 days or at longer intervals than about once every ten days, about once every 15 days or at longer intervals than about once every 15 days, about once every 20 days or at longer intervals than once about every 20 days, about once every 25 days or at longer intervals than about once every 25 days, about once every month or at longer intervals than about once a month, or about once every two months or at longer intervals than about once every two months.

50. A recombinant protein comprising:
(a) an alpha-1-antitrypsin serpin domain and
(b) a human serum albumin domain;
wherein the alpha-1-antrypsin serpin domain and the human serum albumin are not wild-type alpha-1-antrypsin and human serum albumin.


SEQUENCE LISTING (71 sequences)

SEQ ID NO: 1 - protein, 394 aa, synthetic construct
  REGION 1..394: note = Synthetic: Alpha-1-antitrypsin
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 2 - protein, 24 aa, synthetic construct
  REGION 1..24: note = Synthetic: Alpha-1-antitrypsin leader
MPSSVSWGILLLAGLCCLVPVSLA

SEQ ID NO: 3 - protein, 498 aa, Homo sapiens
  REGION 1..498: note = misc_feature - Human Albumin
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQGLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVGSKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDCLSVFLNQLCVLHEKTPVSDRVT

SEQ ID NO: 4 - protein, 18 aa, Homo sapiens
  REGION 1..18: note = misc_feature - Human Albumin Leader
MKWVTFISLLFLFSSAYS

SEQ ID NO: 5 - protein, 480 aa, Homo sapiens
  REGION 1..480: note = misc_feature - Human Serum Albumin
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQGLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVGSKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDCLSVFLNQLCVLHEKTPVSDRVT

SEQ ID NO: 6 - protein, 5 aa, synthetic construct
  REGION 1..5: note = Synthetic: CDR1
LNLMG

SEQ ID NO: 7 - protein, 5 aa, synthetic construct
  REGION 1..5: note = Synthetic: CDR1
INLLG

SEQ ID NO: 8 - protein, 5 aa, synthetic construct
  REGION 1..5: note = Synthetic: CDR1
SFGMS

SEQ ID NO: 9 - protein, 5 aa, synthetic construct
  REGION 1..5: note = Synthetic: CDR1
NYWMY

SEQ ID NO: 10 - protein, 17 aa, synthetic construct
  REGION 1..17: note = Synthetic: CDR2
TCITVGDSTNYADSVKG

SEQ ID NO: 11 - protein, 16 aa, synthetic construct
  REGION 1..16: note = Synthetic: CDR2
TITVGDSTSYADSVKG

SEQ ID NO: 12 - protein, 17 aa, synthetic construct
  REGION 1..17: note = Synthetic: CDR2
SISGSGSDTLYADSVKG

SEQ ID NO: 13 - protein, 17 aa, synthetic construct
  REGION 1..17: note = Synthetic: CDR2
SINGRGDDTRYADSVKG

SEQ ID NO: 14 - protein, 17 aa, synthetic construct
  REGION 1..17: note = Synthetic: CDR2
AISADSSTKNYADSVKG

SEQ ID NO: 15 - protein, 17 aa, synthetic construct
  REGION 1..17: note = Synthetic: CDR2
AISADSSDKRYADSVKG

SEQ ID NO: 16 - protein, 18 aa, synthetic construct
  REGION 1..18: note = Synthetic: CDR2
RDISTGGGYSYYADSVKG

SEQ ID NO: 17 - protein, 8 aa, synthetic construct
  REGION 1..8: note = Synthetic: CDR3
RRTWHSEL

SEQ ID NO: 18 - protein, 6 aa, synthetic construct
  REGION 1..6: note = Synthetic: CDR3
GGSLSR

SEQ ID NO: 19 - protein, 7 aa, synthetic construct
  REGION 1..7: note = Synthetic: CDR3
GRSVSRS

SEQ ID NO: 20 - protein, 5 aa, synthetic construct
  REGION 1..5: note = Synthetic: CDR3
GRGSP

SEQ ID NO: 21 - protein, 13 aa, synthetic construct
  REGION 1..13: note = Synthetic: CDR3
DREAQVDTLDFDY

SEQ ID NO: 22 - protein, 117 aa, synthetic construct
  REGION 1..117: note = Synthetic: SDA
AVQLVESGGGLVQGGGSLRLACAASERIFDLNLMGWYRQGPGNERELVATCITVGDSTNYADSVKGRFTISMDYTKQTVYLHMNSLRPEDTGLYYCKIRRTWHSELWGQGTQVTVSS

SEQ ID NO: 23 - protein, 116 aa, synthetic construct
  REGION 1..116: note = Synthetic: SDA
EVQLVESGGGLVQEGGSLRLACAASERIWDINLLGWYRQGPGNERELVATITVGDSTSYADSVKGRFTISRDYDKNTLYLQMNSLRPEDTGLYYCKIRRTWHSELWGQGTQVTVSS

SEQ ID NO: 24 - protein, 115 aa, synthetic construct [sequence omitted]

SEQ ID NO: 25 - protein, 116 aa, synthetic construct
  REGION 1..116: note = Synthetic: SDA
AVQLVDSGGGLVQPGGSLRLSCAASGFSFGSFGMSWVRQYPGKEPEWVSSINGRGDDTRYADSVKGRFSISRDNAKNTLYLQMNSLKPEDTAEYYCTIGRSVSRSRTQGTQVTVSS

SEQ ID NO: 26 - protein, 114 aa, synthetic construct
  REGION 1..114: note = Synthetic: SDA
AVQLVESGGGLVQPGGSLRLTCTASGFTFRSFGMSWVRQAPGKDQEWVSAISADSSTKNYADSVKGRFTISRDNAKKMLYLEMNSLKPEDTAVYYCVIGRGSPSSPGTQVTVSS

SEQ ID NO: 27 - protein, 114 aa, synthetic construct
  REGION 1..114: note = Synthetic: SDA
QVQLAESGGGLVQPGGSLRLTCTASGFTFGSFGMSWVRQAPGEGLEWVSAISADSSDKRYADSVKGRFTISRDNAKKMLYLEMNSLKSEDTAVYYCVIGRGSPASQGTQVTVSS

SEQ ID NO: 28 - protein, 123 aa, synthetic construct
  REGION 1..123: note = Synthetic: SDA
QVQLVESGGGLVQPGGSLRLSCAASGFTFSNYWMYWVRVAPGKGLERISRDISTGGGYSYYADSVKGRFTISRDNAKNTLYLQMNSLKPEDTALYYCAKDREAQVDTLDFDYRGQGTQVTVSS

SEQ ID NO: 29 - protein, 115 aa, synthetic construct [sequence omitted]

SEQ ID NO: 30 - protein, 115 aa, synthetic construct [sequence omitted]

SEQ ID NO: 31 - protein, 115 aa, synthetic construct [sequence omitted]

SEQ ID NO: 32 - protein, 115 aa, synthetic construct [sequence omitted]

SEQ ID NO: 33 - protein, 115 aa, synthetic construct [sequence omitted]

SEQ ID NO: 34 - protein, 115 aa, synthetic construct [sequence omitted]

SEQ ID NO: 35 - protein, 115 aa, synthetic construct [sequence omitted]

SEQ ID NO: 36 - protein, 115 aa, synthetic construct [sequence omitted]

SEQ ID NO: 37 - protein, 30 aa, synthetic construct
  REGION 1..30: note = Synthetic: FR1
AVQLVESGGGLVQGGGSLRLACAASERIFD

SEQ ID NO: 38 - protein, 30 aa, synthetic construct
  REGION 1..30: note = Synthetic: FR1
EVQLVESGGGLVQEGGSLRLACAASERIWD

SEQ ID NO: 39 - protein, 30 aa, synthetic construct
  REGION 1..30: note = Synthetic: FR1
AVQLVESGGGLVQPGNSLRLSCAASGFTFR

SEQ ID NO: 40 - protein, 30 aa, synthetic construct
  REGION 1..30: note = Synthetic: FR1
AVQLVDSGGGLVQPGGSLRLSCAASGFSFG

SEQ ID NO: 41 - protein, 30 aa, synthetic construct
  REGION 1..30: note = Synthetic: FR1
AVQLVESGGGLVQPGGSLRLTCTASGFTFR

SEQ ID NO: 42 - protein, 30 aa, synthetic construct
  REGION 1..30: note = Synthetic: FR1
QVQLAESGGGLVQPGGSLRLTCTASGFTFG

SEQ ID NO: 43 - protein, 30 aa, synthetic construct
  REGION 1..30: note = Synthetic: FR1
QVQLVESGGGLVQPGGSLRLSCAASGFTFS

SEQ ID NO: 44 - protein, 14 aa, synthetic construct
  REGION 1..14: note = Synthetic: FR2
WYRQGPGNERELVA

SEQ ID NO: 45 - protein, 14 aa, synthetic construct
  REGION 1..14: note = Synthetic: FR2
WVRQAPGKEPEWVS

SEQ ID NO: 46 - protein, 14 aa, synthetic construct
  REGION 1..14: note = Synthetic: FR2
WVRQYPGKEPEWVS

SEQ ID NO: 47 - protein, 14 aa, synthetic construct
  REGION 1..14: note = Synthetic: FR2
WVRQAPGKDQEWVS

SEQ ID NO: 48 - protein, 14 aa, synthetic construct
  REGION 1..14: note = Synthetic: FR2
WVRQAPGEGLEWVS

SEQ ID NO: 49 - protein, 14 aa, synthetic construct
  REGION 1..14: note = Synthetic: FR2
WVRVAPGKGLERIS

SEQ ID NO: 50 - protein, 32 aa, synthetic construct
  REGION 1..32: note = Synthetic: FR3
RFTISMDYTKQTVYLHMNSLRPEDTGLYYCKI

SEQ ID NO: 51 - protein, 32 aa, synthetic construct
  REGION 1..32: note = Synthetic: FR3
RFTISRDYDKNTLYLQMNSLRPEDTGLYYCKI

SEQ ID NO: 52 - protein, 32 aa, synthetic construct
  REGION 1..32: note = Synthetic: FR3
RFTISRDNAKTTLYLQMNSLKPEDTAVYYCTI

SEQ ID NO: 53 - protein, 32 aa, synthetic construct
  REGION 1..32: note = Synthetic: FR3
RFSISRDNAKNTLYLQMNSLKPEDTAEYYCTI

SEQ ID NO: 54 - protein, 32 aa, synthetic construct
  REGION 1..32: note = Synthetic: FR3
RFTISRDNAKKMLYLEMNSLKPEDTAVYYCVI

SEQ ID NO: 55 - protein, 32 aa, synthetic construct
  REGION 1..32: note = Synthetic: FR3
RFTISRDNAKKMLYLEMNSLKSEDTAVYYCVI

SEQ ID NO: 56 - protein, 32 aa, synthetic construct
  REGION 1..32: note = Synthetic: FR3
RFTISRDNAKNTLYLQMNSLKPEDTALYYCAK

SEQ ID NO: 57 - protein, 11 aa, synthetic construct
  REGION 1..11: note = Synthetic: FR4
WGQGTQVTVSS

SEQ ID NO: 58 - protein, 11 aa, synthetic construct
  REGION 1..11: note = Synthetic: FR4
SSQGTQVTVSS

SEQ ID NO: 59 - protein, 11 aa, synthetic construct
  REGION 1..11: note = Synthetic: FR4
RTQGTQVTVSS

SEQ ID NO: 60 - protein, 11 aa, synthetic construct
  REGION 1..11: note = Synthetic: FR4
SSPGTQVTVSS

SEQ ID NO: 61 - protein, 11 aa, synthetic construct
  REGION 1..11: note = Synthetic: FR4
ASQGTQVTVSS

SEQ ID NO: 62 - protein, 11 aa, synthetic construct
  REGION 1..11: note = Synthetic: FR4
RGQGTQVTVSS

SEQ ID NO: 63 - protein, 14 aa, synthetic construct
  REGION 1..14: note = Synthetic: Linker
GGSGGSGGSGGSGG

SEQ ID NO: 64 - protein, 1017 aa, synthetic construct
  REGION 1..1017: note = Synthetic: AAT-linkerAlbumin Fusion
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKGGSGGSGGSGGSGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 65 - protein, 1017 aa, synthetic construct
  REGION 1..1017: note = Synthetic: Mutant AAT-linkerAlbumin Fusion (C232S)
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHSKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKGGSGGSGGSGGSGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 66 - protein, 1017 aa, synthetic construct
  REGION 1..1017: note = Synthetic: Mutant AAT-linkerAlbumin Fusion (M351V, M358V)
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAVFLEAIPVSIPPEVKFNKPFVFLMIEQNTKSPLFMGKVVNPTQKGGSGGSGGSGGSGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 67 - protein, 1017 aa, synthetic construct
  REGION 1..1017: note = Synthetic: Mutant AAT-linkerAlbumin Fusion (M351V, M358L)
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAVFLEAIPLSIPPEVKFNKPFVFLMIEQNTKSPLFMGKVVNPTQKGGSGGSGGSGGSGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 68 - protein, 1017 aa, synthetic construct
  REGION 1..1017: note = Synthetic: Mutant AAT-linkerAlbumin Fusion (C232S, M351V, M358L)
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHSKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAVFLEAIPLSIPPEVKFNKPFVFLMIEQNTKSPLFMGKVVNPTQKGGSGGSGGSGGSGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 69 - DNA, 3178 bp, Homo sapiens
  misc_feature 1..3178: note = Wild Type AAT Nucleic Acid
  source 1..3178: mol_type = unassigned DNA
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaaggacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgaattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 70 - DNA, 19002 bp, Homo sapiens
  misc_feature 1..19002: note = Wild Type Human Serum Albumin Nucleic Acid
  source 1..19002: mol_type = unassigned DNA
cctttcccagggacttctacaaggaaaaagctagagttggttactgacttctaataaataatgcctacaatttctaggaagttaaaagttgacataatttatccaagaaagaattattttcttaacttagaatagtttcttttttcttttcagatgtaggtttttctggctttagaaaaaatgcttgtttttcttcaatggaaaataggcacacttgttttatgtctgttcatctgtagtcagaaagacaagtctggtatttcctttcaggactcccttgagtcattaaaaaaaatcttcctatctatctatgtatctatcatccatctagctttgattttttcctcttctgtgctttattagttaattagtacccatttctgaagaagaaataacataagattatagaaaataatttctttcattgtaagactgaatagaaaaaattttctttcattataagactgagtagaaaaaataatactttgttagtctctgtgcctctatgtgccatgaggaaatttgactactggttttgactgactgagttatttaattaagtaaaataactggcttagtactaattattgttctgtagtatcagagaaagttgttcttcctactggttgagctcagtagttcttcatattctgagcaaaagggcagaggtaggatagcttttctgaggtagagataagaaccttgggtagggaaggaagatttatgaaatatttaaaaaattattcttccttcgctttgtttttagacataatgttaaatttattttgaaatttaaagcaacataaaagaacatgtgatttttctacttattgaaagagagaaaggaaaaaaatatgaaacagggatggaaagaatcctatgcctggtgaaggtcaagggttctcataacctacagagaatttggggtcagcctgtcctattgtatattatggcaaagataatcatcatctcatttgggtccattttcctctccatctctgcttaactgaagatcccatgagatatactcacactgaatctaaatagcctatctcagggcttgaatcacatgtgggccacagcaggaatgggaacatggaatttctaagtcctatcttacttgttattgttgctatgtctttttcttagtttgcatctgaggcaacatcagctttttcagacagaatggctttggaatagtaaaaaagacacagaagccctaaaatatgtatgtatgtatatgtgtgtgtgcatgcgtgagtacttgtgtgtaaatttttcattatctataggtaaaagcacacttggaattagcaatagatgcaatttgggacttaactctttcagtatgtcttatttctaagcaaagtatttagtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcacaatgaagtgggtaacctttatttcccttctttttctctttagctcggcttattccaggggtgtgtttcgtcgagatgcacgtaagaaatccatttttctattgttcaacttttattctattttcccagtaaaataaagttttagtaaactctgcatctttaaagaattattttggcatttatttctaaaatggcatagtattttgtatttgtgaagtcttacaaggttatcttattaataaaattcaaacatcctaggtaaaaaaaaaaaaaggtcagaattgtttagtgactgtaattttcttttgcgcactaaggaaagtgcaaagtaacttagagtgactgaaacttcacagaatagggttgaagattgaattcataactatcccaaagacctatccattgcactatgctttatttaaaaaccacaaaacctgtgctgttgatctcataaatagaacttgtatttatatttattttcattttagtctgtcttcttggttgctgttgatagacactaaaagagtattagatattatctaagtttgaatataaggctataaatatttaataatttttaaaatagtattcttggtaattgaattattcttctgtttaaaggcagaagaaataattgaacatcatcctgagtttttctgtaggaatcagagcccaatattttgaaacaaatgcataatctaagtcaaatggaaagaaatataaaaagtaacattattacttcttgttttcttcagtatttaacaatccttttttttcttcccttgcccagacaagagtgaggttgctcatcggtttaaagatttgggagaagaaaatttcaaagccttgtaagttaaaatattgatgaatcaaatttaatgtttctaatagtgttgtttattattctaaagtgcttatatttccttgtcatcagggttcagattctaaaacagtgctgcctcgtagagttttctgcgttgaggaagatattctgtatctgggctatccaataaggtagtcactggtcacatggctattgagtacttcaaatatgacaagtgcaactgagaaacaaaaacttaaattgtatttaattgtagttaatttgaatgtatatagtcacatgtggctaatggctactgtattggacagtacagctctggaacttgcttggtggaaaggactttaatataggtttcctttggtggcttacccactaaatcttctttacatagcaagcattcctgtgcttagttgggaatatttaatttttttttttttttaagacagggtctcgctctgtcgcccaggctggagtgcagtggcgcaatctcggctcactgcaaactccgctcccgggttcacgccattctcctgcctcagcctcccgagtagctgggactacaggcgcccgccatcacgcccggctaatcttttgtatttttagtagagatggggtttcaccgtgtgccaggatggtctcaatctcctgacatcgtgatctgcccacctcggcctcccaaagtgctgggattacaggagtgagtcaccgcgcccggcctatttaaatgttttttaatctagtaaaaaatgagaaaattgtttttttaaaagtctacctaatcctacaggctaattaaagacgtgtgtggggatcaggtgcggtggttcacacctgtaatcccagcactttggaaggctgatgcaggaggattgcttgagcccaggagtacaagaccagcctgggcaagtctctttaaaaaaaacaaaacaaacaaacaaaaaaattaggcatggtggcacatgcctgtagtcctagctacttaggaggctgacgtaggaggatcgtttggacctgagaggtcaaggctacagtgagccatgattgtgccactgcactccagcctgggtgacagagtgagactctgtctcaaaaaagaaaaaggaaatctgtggggtttgttttagttttaagtaattctaaggactttaaaaatgcctagtcttgacaattagatctatttggcatacaatttgcttgcttaatctatgtgtgtgcatagatctactgacacacgcatacatataaacattagggaactaccattctctttgcgtaggaagccacatatgcctatctaggcctcagatcatacctgatatgaataggctttctggataatggtgaagaagatgtataaaagatagaacctatacccatacatgatttgttctctagcgtagcaacctgttacatattaaagttttattatactacatttttctacatcctttgtttcagggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttgtaagtacattctaattgtggagattctttcttctgtttgaagtaatcccaagcatttcaaaggaattttttttaagttttctcaattattattaagtgtcctgatttgtaagaaacactaaaaagttgctcatagactgataagccattgtttcttttgtgatagagatgctttagctatgtccacagttttaaaatcatttctttattgagaccaaacacaacagtcatggtgtatttaaatggcaatttgtcatttataaacacctctttttaaaatttgaggtttggtttctttttgtagaggctaatagggatatgatagcatgtatttatttatttatttatcttattttattatagtaagaacccttaacatgagatctaccctgttatatttttaagtgtacaatccattattgttaactacgggtacactgttgtatagcttactcatcttgctgtattaaaactttgtgcccattgattagtaacccctcgtttcgtcctcccccagccactggcaaccagcattatactctttgattctatgagtttgactactttagctaccttatataagtggtattatgtactgtttatctttttatgactgacttatttcccttagcatagtgcattcaaagtccaaccatgttgttgcctattgcagaatttccttcttttcaaggctgaataatattccagtgcatgtgtgtaccacattttctttatccattaatttgttgattgatagacatttaggttggttttctacatcttgactatcatgaatagtgttgcaatgaacacaggagagctactatctcttagagatgatatcatggtttttatcatcagaaaacacccactgatttctatgctaattttgttacctgggtggaataatagtacagctatatattcctcattttagatatctttgtatttctacatacaataaaaaagcagagtacttagtcatgttgaagaactttaaacttttagtatttccagatcaatcttcaaaacaaggacaggtttatctttctctcaccactcaatctatatatacctcttgtgggcaaggccagtttttatcactggagcctttcccctttttattatgtacctctccctcacagcagagtcaggactttaactttacacaatactatggctctacatatgaaatcttaaaaatacataaaaattaataaattctgtctagagtagtatattttccctggggttacgattactttcataataaaaattagagataaggaaaggactcatttattggaaagtgattttaggtaacatttctggaagaaaaatgtctatatcttaatagtcacttaatatatgatggattgtgttactcctcagttttcaatggcatatactaaaacatggccctctaaaaagggggcaaatgaaatgagaaactctctgaatgtttttctcccctaggtgaattcacctgctgcttagaagcttattttctcttgatttctgttataatgattgctcttaccctttagttttaagtttcaaaataggagtcatataactttccttaaagctattgactgtctttttgtcctgttttattcaccatgagttatagtgtgacagttaattcttatgaaaattatatagagatggttaaatcatcagaaactgtaaacctcgattgggaggggaagcggatttttaaatgatttcctgaccaagcttaaccagtatattaaatcctttgtactgttctttggctataaagaaaaaaggtactgtccagcaactgaaacctgctttcttccatttagcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaagtaagtaatcagatgtttatagttcaaaattaaaaagcatggagtaactccataggccaacactctataaaaattaccataacaaaaatattttcaacattaagacttggaagttttgttatgatgattttttaaagaagtagtatttgataccacaaaattctacacagcaaaaaatatgatcaaagatattttgaagtttattgaaacaggatacaatctttctgaaaaatttaagatagacaaattatttaatgtattacgaagatatgtatatatggttgttataattgatttcgttttagtcagcaacattatattgccaaaatttaaccatttatgcacacacacacacacacacacacacttaacccttttttccacatacttaaagaatgacagagacaagaccatcatgtgcaaattgagcttaattggttaattagatatctttggaatttggaggttctggggagaatgtcgattacaattatttctgtaatattgtctgctatagaaaagtgactgtttttctttttcaaaatttagatacttatatgaaattgccagaagacatccttacttttatgccccggaactccttttctttgctaaaaggtataaagctgcttttacagaatgttgccaagctgctgataaagctgcctgcctgttgccaaaggtattatgcaaaagaatagaaaaaaagagttcattatccaacctgattttgtccattttgtggctagatttagggaacctgagtgtctgatacaaactttccgacatggtcaaaaaagccttccttttatctgtcttgaaaatctttcatctttgaaggcctacactctcgtttcttcttttaagatttgccaatgatgatctgtcagaggtaatcactgtgcatgtgtttaaagatttcaccactttttatggtggtgatcactatagtgaaatactgaaacttgtttgtcaaattgcacagcaaggggacacagttcttgtttatcttttcatgataatttttagtagggagggaattcaaagtagagaattttactgcatctagatgcctgagttcatgcattcattccataaatatatattatggaatgctttattttcttttctgaggagtttactgatgttggtggaggagagactgaaatgaattatacacaaaatttaaaaattagcaaaattgcagcccctgggatattagcgtactctttctctgacttttctcccacttttaaggctctttttcctggcaatgtttccagttggtttctaactacatagggaattccgctgtgaccagaatgatcgaatgatctttccttttcttagagagcaaaatcattattcgctaaagggagtacttgggaatttaggcataaattatgccttcaaaatttaatttggcacagtctcatctgagcttatggaggggtgtttcatgtagaatttttcttctaattttcatcaaattattcctttttgtagctcgatgaacttcgggatgaagggaaggcttcgtctgccaaacagagactcaagtgtgccagtctccaaaaatttggagaaagagctttcaaagcatggtaaatacttttaaacatagttggcatctttataacgatgtaaatgataatgcttcagtgacaaattgtacatttttatgtattttgcaaagtgctgtcaaatacatttctttggttgtctaacaggtagaactctaatagaggtaaaaatcagaatatcaatgacaatttgacattatttttaatcttttcttttctaaatagttgaataatttagaggacgctgtcctttttgtcctaaaaaaagggacagatatttaagttctatttatttataaaatcttggactcttattctaatggttcattatttttatagagctgtaggcatggttctttatttaattttttaaagttatttttaatttttgtggatacagagtaggtatacatatttacggggtatatgagatattttgatataagtatacaacatatataatccctttatttaattttatcttccccccaatgatctaaaactatttgcttgtccttttatgtcttatagttaaattcagtcaccaactaagttgaagttacttcttatttttgcatagctccagctctgatcttcatctcatgtttttgcctgagcctctgttttcatattacttagttggttctgggagcatactttaatagccgagtcaagaaaaatactagctgccccgtcacccacactcctcacctgctagtcaacagcaaatcaacacaacaggaaataaaatgaaaataatagacattatgcatgctctctagaaactgtcaattgaactgtatttgctcatcattcctaccatctacaccaccaaaatcaaccaaatttatgaaaaaaaaacagccccaacataaaattatacacagataaacaggctatgattggttttgggaaagaagtcacctttacctgatttaggcaactgtgaaatgactagagaatgaagaaaattagacgtttacatcttgtcatagagtttgaagatagtgctggatctttctttttataagtaagatcaataaaaactccctcattctgtagaagttatgatttcttttctaagagacctttagaagtcagaaaaaatgtgtttcaattgagaaaaaagataactggagtttgtgtagtacttcccagattataaaatgcttttgtatgtattatctaatttaatcctcaaaacttcttcaatttagcatgttgtcatgacactgcagaggctgaagctcagagacgctgagccctctgctaacaagtcctactgctaacaagtgataaagccagagctggaagtcacatctggactccaaacctgatgcttctcagcctgttgccccttttagagttcctttttaatttctgcttttatgacttgctagatttctacctaccacacacactcttaaatggataattctgccctaaggataagtgattaccatttggttcagaactagaactaatgaattttaaaaattatttctgtatgtccattttgaattttcttatgagaaatagtatttgcctagtgttttcatataaaatatcgcatgataataccattttgattggcgattttctttttagggcagtagctcgcctgagccagagatttcccaaagctgagtttgcagaagtttccaagttagtgacagatcttaccaaagtccacacggaatgctgccatggagatctgcttgaatgtgctgatgacagggtaaagagtcgtcgatatgctttttggtagcttgcatgctcaagttggtagaatggatgcgtttggtatcattggtgatagctgacagtgggttgagattgtcttctgtgctttcgtctgtcctatcttcaatctttccctgcctatggtggtggtacctttctgtttttaacctgctataaattaccagataaacccattcactgatttgtaactcctttcagtcatgctctaactgtaaatgaaggcttaaactgaagtagaacagttacaaggttttacttggcagaacatcttgcaaggtagatgtctaagaagatttttttttctttttttaagacagagtttcgctcttgtttcccaggctggggtgcaatggtgtgatcttggctcagcgcaacctctgcctcctgggttcaagtgattttcatgcctcagcctcccaagtagctgggattacaggcatgcgccaccacacctggctaattttgtatttttagtagaggcggggtttcaccatattgtccagactggtctcgaactcctgacctcaggtgatccacccgccttggcctcccaaagtgctgggattacaggcatgagccaccttgcccagcctaagaagattttttgagggaggtaggtggacttggagaaggtcactacttgaagagatttttggaaatgatgtatttttcttctctatattccttcccttaattaactctgtttgttagatgtgcaaatatttggaatgatatctcttttctcaaaacttataatattttctttctccctttcttcaagattaaacttatgggcaaatactagaatcctaatctctcatggcactttctggaaaatttaaggcggttattttatatatgtaagcagggcctatgactatgatcttgactcatttttcaaaaatcttctatattttatttagttatttggtttcaaaaggcctgcacttaattttgggggattatttggaaaaacagcattgagttttaatgaaaaaaacttaaatgccctaacagtagaaacataaaattaataaataactgagctgagcacctgctactgattagtctattttaattaagtgggaatgtttttgtagtcctatctacatctccaggtttaggagcaaacagagtatgttcatagaaggaatatgtgtatggtcttagaatacaatgaacatgttctgccaacttaataaaggtctgaggagaaagtgtagcaatgtcaattcgtgttgaacaatttccaccaacttacttataggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtaagtagataagaaattattcttttatagctttggcatgacctcacaacttaggaggatagcctaggcttttctgtggagttgctacaatttccctgctgcccagaatgtttcttcatccttccctttcccaggctttaacaatttttgaaatagttaattagttgaatacattgtcataaaataatacatgttcacggcaaagctcaacattccttactccttaggggtatttctgaaaatacgtctagaaacattttgtgtatatataaattatgtatacttcagtcattcattccaagtgtatttcttgaacatctataatatatgtgtgtgactatgtattgcctgtctatctaactaatctaatctaatctagtctatctatctaatctatgcaatgatagcaaagaagtataaaaagaaatatagagtctgacacaggtgctttatatttggtgaaaagaccagaagttcagtataatggcaatatggtaggcaactcaattacaaaataaatgtttacgtattgtcagaagttgtggtgataaactgcatttttgttgttggattatgataatgcactaaataatatttcctaaaattatgtaccctacaagatttcactcatacagagaagaaagagaatattttaagaacatatctctgcccatctatttatcagaatccttttgagatgtagtttaaatcaaacaaaatgttaataaaaataacaagtatcattcatcaaagacttcatatgtgccaagcagtgtgtgctttgtgtagattatgtcatatagttctcataatccaccttccgagacagatactatttattttttgagacagagttttactcttgttgcccaggctggagtgcaatggtgccatctcggctcaccacaaccttcgcctcccaggttcaagcgattctcctgcctcagcctcctgggattacaggcatgcaccaccatgcctggctaattttgtatttttagtagagatggggtttcaccatgttggtcagactggtctcaaactcctgacctctggtgatatgcctgcctcagcctcctaaagtgctgggattacaggcatgagccactgtgcccagccgacagatactattattatttccattctaccgagaaggagactaaggctctgatcatttaaataagttgcctaaggtgatgcagtgatataagtagcagagctaggaattgagccttggtaactttaactctggaccccaagtccttagctactaagctttactgcatggggtttagtcaaattaagacttttggaatatgagttacttttgagattagctttgtgatattttttgtgctcatttgtccaacaaagtctattttattttcatcttaattaggtttttgtatgaatatgcaagaaggcatcctgattactctgtcgtgctgctgctgagacttgccaagacatatgaaaccactctagagaagtgctgtgccgctgcagatcctcatgaatgctatgccaaagtggtaggtttattgttggaaaaaaatgtagttctttgactgatgattccaataatgagaaagaaaaataatgcaagaatgtaaaatgatatacagtgcaatttagatcttttcttgagatggtttcaattctggaatcttaaacatgaaagaaaaagtagccttagaatgattaacaaaatttagactagttagaatagaaagatctgaatagagcaatctctaaaaaattttgatctttttttctctttttcacaatcctgagaacaaaaaaaaattaaatttaaatgttaattagaagatatttaacttagatgtaaagtgagttaacctgattccaggattaatcaagtactagaattagtatcttatggcaaattatagaacctatccctttagaatattttcaaatctttttgaggatgtttaggaatagttttacaagaaattaagttaggagaggaaatctgttctggaggatttttagggttcccactagcatatgtaatggtttctgaactattcagaatcagagaaaactcatttttcctgctttcaagaagctactgtatgccaggcaccatgcacaaacaatgaccaacgtaaaatctctcattttggagagcctggaatctaactggaaaggtgaactaataataataatatgtacaatcatagccatcatttattaaacttttattatatgcaaggcactgtttaatttcattagcttacctggtttacagagcagctctatgagatgagtgccatctttgcccctattttagggataaggattccgaaatgtggagatggtaagtaaaattgcacaactgaagaatgagttacatgacttggctcaaatactggtcattgaactccagagcctgaatattcttaaccacttacatgatgcaagctcaccaaataaatagttcgaatgtattgtgacagagcggcattgatattcatctattcatgtggctttgagtaggaagaagaaaggatatcattctgaccagaggggtgaaaaacaacctgcatctgatcctgaggcataatactattaacacaattcttttatgtttcagttcgatgaatttaaacctcttgtggaagagcctcagaatttaatcaaacaaaattgtgagctttttgagcagcttggagagtacaaattccagaatgcgtaagtaatttttattgactgattttttttatcaatttgtaattatttaagacttaatatatgagccacctagcatagaacttttaagaatgaaaatacattgcatatttctaatcactctttgtcaagaaagataggagaggagagataaaatagttgatggggtggagaggtctatatttgaatgtagtctaaaaattgttctcttaagattggaagtatgtaggctgggagggtaaataccaaatcttggtatatcagaactgagcatgtcccttgaaggttaagaaatagttaatgggcaaatagagcatggcaatattttgtagagcagcaagtagtaggccttgaatagatgtcgctcaaaaagtaatatgtaagctgaacacaaaaatgtaacaaatgaatttagatacatatttgaatattaaattcaggttgtttgggagatgcacctagtctttgatggttaaacctttccctccatagaagagacagagacagaatggcttgctggactaatgtcccaattcaatagagtcttatctacgaaggttaaaaacaagaagagacatattatacagtagatatttattgtgtggctcatacacatggtgctcttctgattatggattttagagataataacagtgaacaagacatagtttctttcctcgagtagattaaagtcatacattgacttttaatggtgactggcattcttaatacatgattattatatattaggtaccatgtcagattaattataatactttactatttttaatttaacccttgaactatccctattgagtcagatatatttccttccattttctacttgtatctttcaagtttagcatatgctgatacatatgaagctctctccaggttttattgaaagaagaaattaataaatttattaatgtcactgaattaggcaactcactttcccaagattatgcaagtggtacaggtggaactcaaagccaagtttaactagttgttcaggagaatgttttctaccctccactaacccactactctgcagatggagataatatgatgaatggaacatagcaacatcttagttgattccggccaagtgttctctgttttatctactatgttagacagtttcttgccttgctgaaaacacatgacttctttttttcaggctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatgtgagtctttaaaaaaatataataaattaataatgaaaaaattttacctttagatattgataatgctagctttcataagcagaaggaagtaatgtgtgtgtgtgcatgtttgtgtgcatgtgtgtgtgcatgcacgtgtgtgtatgtgtgatattggcagtcaaggccccgaggatgataattttttttttttttttgagacggagtctcgctttgttgtccaggctggagtgcagtggtgccatctcggctcactgcaacctccgcctcccaagttcaagccattctcctgcctcagcctcccaagtagctgggactacaggtgcatgccaccatgcctggctaattttttgtatttttagtagaaaattttcagcttcacctcttttgaatttctgctctcctgcctgttctttagctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaacgtgaggagtatttcattactgcatgtgtttgtagtcttgatagcaagaactgtcaattcaagctagcaactttttcctgaagtagtgattatatttcttagaggaaagtattggagtgttgcccttattatgctgataagagtacccagaataaaatgaataactttttaaagacaaaatcctctgttataatattgctaaaattattcagagtaatattgtggattaaagccacaatagaataacatgttagaccatattcagtagaaaaagatgaacaattaactgataaatttgtgcacatggcaaattagttaatgggaaccataggagaatttatttctagatgtaaataattattttaagtttgccctatggtggccccacacatgagacaaacccccaagatgtgacttttgagaatgagacttggataaaaaacatgtagaaatgcaagccctgaagctcaactccctattgctatcacaggggttataattgcataaaatttagctatagaaagttgctgtcatctcttgtgggctgtaatcatcgtctaggcttaagagtaatattgcaaaacctgtcatgcccacacaaatctctccctggcattgttgtctttgcagatgtcagtgaaagagaaccagcagctcccatgagtttggatagccttattttctatagcctccccactgaagggagcaaagtttaagaaccaaatataaagtttctcatctttatagatgagaaaaattttaaataaagtccaagataattaaatttttaaggatcatttttagctctttaatagcaataaaactcaatatgacataatatggcacttccaaaatctgaataatatataattgcaatgacatacttcttttcagagatttactgaaaagaaatttgttgacactacataacgtgatgagtggtttatactgattgtttcagttggtcttcccaccaactccatgaaagtggattttattatcctcatcatgcagatgagaatattgagacttatagcggtatgcctggcccaagtactcagagttgcctggctccaagatttataatcttaaatgatgggactaccatccttactctctccatttttctatacgtgagtaatgttttttctgttttttttttttctttttccattcaaactcagtgcacttgttgagctcgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttgaatgtttataatgcatgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaatttccctttatggcctgacagtaacttttttttattcatttggggacaactatgtccgtgagcttccatccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttataacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaggtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaagttatggattataaacattcaaaataatattttgacattatgataattctgaataaaagaacaaaaaccatggtataggtaaggaatataaaacatggcttttaccttagaaaaaacaattctaaaattcatatggaatcaaaaaagagcctgcag

SEQ ID NO: 71 - protein, 993 aa, synthetic construct
  REGION 1..993: note = Synthetic: Mutant AAT-linkerAlbumin Fusion (S283C, K335A, P361C)
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHSKKLSSWVLLMKYLGNATAIFFLPDEGKLQHLENELTHDIITKFLENEDRRCASLHLPKLSITGTYDLKSVLGQLGITKVFSNGADLSGVTEEAPLKLSKAVHAAVLTIDEKGTEAAGAVFLEAIPLSICPEVKFNKPFVFLMIEQNTKSPLFMGKVVNPTQKGGSGGSGGSGGSGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL